(19) United States
(12) Patent Application Publication
(10) Pub. No.: US 2008/0145427 A1
(43) Pub. Date: Jun. 19, 2008

(54) Dosage Forms Providing Controlled and Immediate Release of Cholesteryl Ester Transfer Protein Inhibitors and Immediate Release of Hmg-Coa Reductase Inhibitors

(76) Inventors: Alfred Berchielli, Niantic, CT (US); Eric K. Eisenhart, Waterford, CT (US); Scott M. Herbig, East Lyme, CT (US); Barbara A. Johnson, Niantic, CT (US)

Correspondence Address: PFIZER INC., PATENT DEPARTMENT, MS8260-1611, EASTERN POINT ROAD, GROTON, CT, 06340, US

(21) Appl. No.: 11/814,712

(22) PCT Filed: Jan. 23, 2006
(86) PCT No.: PCT/IB06/00192

§ 371 Date: Jul. 25, 2007

Related U.S. Application Data
(60) Provisional application No. 60/650,048, filed on Feb. 3, 2005.
(60) Provisional application No. 60/739,220, filed on Nov. 22, 2005.

(51) Int. Cl. A61K 9/00; A61P 9/00; A61K 9/14; A61K 31/47; A61K 31/40

(52) U.S. Cl. 424/484; 424/489; 514/313; 514/423

(57) ABSTRACT

A dosage form that includes a cholesteryl ester transfer protein inhibitor in a solubility-improved form and an HMG-CoA reductase inhibitor, wherein the dosage form provides immediate release of the HMG-CoA reductase inhibitor and controlled release and immediate release of the cholesteryl ester transfer protein inhibitor.

### BACKGROUND OF THE INVENTION

The present invention relates to a dosage form comprising (1) a CETP inhibitor in a solubility-improved form and (2) an HMG-CoA reductase inhibitor, wherein the dosage form provides immediate release of the HMG-CoA reductase inhibitor and controlled release and immediate release of the CETP inhibitor.

It is well known that inhibitors of 3-hydroxy-3-methylglutaryl-coenzyme A reductase (HMG-CoA reductase), an important enzyme catalyzing the intracellular synthesis of cholesterol, will bring about reduced levels of blood cholesterol, especially in terms of the low density lipoprotein form of cholesterol (LDL-C). Therefore, HMG-CoA reductase inhibitors are considered potentially useful as hypocholesterolemic or hypolipidemic agents.

Cholesteryl ester transfer protein inhibitors (CETP inhibitors) are another class of compounds that are capable of modulating levels of blood cholesterol, such as by raising high-density lipoprotein (HDL) cholesterol and lowering low-density lipoprotein (LDL) cholesterol. It is desired to use CETP inhibitors to lower certain plasma lipid levels, such as LDL-cholesterol and triglycerides and to elevate certain other plasma lipid levels, including HDL-cholesterol and accordingly to treat diseases which are affected by low levels of HDL cholesterol and/or high levels of LDL-cholesterol and triglycerides, such as atherosclerosis and cardiovascular diseases in certain mammals (i.e., those which have CETP in their plasma), including humans.

It is well known that a combination therapy of a CETP inhibitor and an HMG-CoA reductase inhibitor may be used to treat elevated LDL cholesterol and low HDL cholesterol levels. For example, WO02/13797 A2 relates to pharmaceutical combinations of cholesteryl ester transfer protein inhibitors and atorvastatin. The application discloses that the compounds may be generally administered separately or together, with a pharmaceutically acceptable carrier, vehicle or diluent. The compounds may be administered individually or together in any conventional oral, parenteral or transdermal dosage form. The combination may be administered in a controlled release dosage formulation, such as a slow release or a fast release formulation. For oral administration, the dosage form may take the form of solutions, suspensions, tablets, pills, capsules, powders and the like.

CETP inhibitors, particularly those that have high binding activity, are generally hydrophobic, have extremely low aqueous solubility and have low oral bioavailability when dosed conventionally. Such compounds have generally proven to be difficult to formulate for oral administration such that high bioavailabilities are achieved. Accordingly, CETP inhibitors must be formulated so as to be capable of providing good bioavailability. Such formulations are generally termed “solubility-improved” forms. One method for increasing the bioavailability of a CETP inhibitor is to form a solid amorphous dispersion of the drug and a concentration-enhancing polymer. See, e.g., commonly assigned, copending U.S. Patent Application No. 2002/010325 A1 and U.S. patent application Ser. No. 10/066,091, the disclosures of which are incorporated herein by reference. Another method for increasing the bioavailability of a CETP inhibitor is to formulate the compound in a lipid vehicle. See commonly assigned, copending U.S. patent application Ser. No. 10/175,643, the disclosures of which are incorporated herein by reference. Additional methods for increasing the bioavailability of a CETP inhibitor include adsorbing the CETP inhibitor onto a porous substrate (see commonly assigned PCT application number WO 03/00238A1), and providing a stabilized amorphous form of a CETP inhibitor with a concentration-enhancing polymer (see commonly assigned PCT application number WO 03/00294A1).

Designing dosage forms with the CETP inhibitor in a solubility-improved form presents further challenges. Use of a solubility-improved form of the CETP inhibitor generally increases the size of the dosage form, e.g. tablet or capsule. It is important that this oral dosage form be of a size that is easily swallowed, particularly for elderly patients. It is also preferable that the number of dosage forms taken per dose be low, preferably one unit, because many patients take multiple drugs. Furthermore, it is important that dosing be convenient, i.e. once-per-day or twice-per-day, because patients who take multiple drugs may have a difficult time keeping track of which drugs to take at which time of day. Furthermore, some drugs such as CETP inhibitors are advantageously taken with a meal, and it is preferable to minimize the number of times per day that the drug is taken, to simplify the requirement that the drug be taken with a meal.

The above references show continuing need to find safe, effective methods of delivering combinations of HMG-CoA reductase inhibitors and CETP inhibitors.

### SUMMARY OF INVENTION

The present invention provides a dosage form comprising (1) a CETP inhibitor in a solubility-improved form and (2) an HMG-CoA reductase inhibitor, wherein the HMG-CoA reductase inhibitor is in immediate release form, a portion of the CETP inhibitor is in immediate release form and a portion of the CETP inhibitor is in controlled release form.

In one embodiment, a portion of the CETP inhibitor can be present in an immediate release form such that at least about 70 wt % of the immediate release portion is released within one hour or less following introduction to a use environment. In another embodiment, the portion of the CETP inhibitor that is in immediate release form should be no greater than about 50% of the entire amount of CETP inhibitor present in the dosage form, preferably no more than about 40%, more preferably no more than about 35%, more preferably no more than about 30%, more preferably no more than about 25%, more preferably no more than about 20%. The immediate release portion of the CETP inhibitor may be accomplished by any means known in the pharmaceutical arts, including immediate release coatings, immediate release layers, and immediate release multiparticulates or granules.

In preferred embodiments, the dosage form releases the HMG-CoA reductase inhibitor and the CETP inhibitor at preferred rates, described herein.

In one embodiment, the CETP inhibitor is in the form of a matrix controlled-release device. The HMG-CoA reductase inhibitor is in the form of an immediate release coating around the matrix controlled-release device, or in the form of an immediate release layer associated with the matrix controlled-release device. An immediate release portion of the CETP inhibitor can also be included in the immediate release coating or layer associated with the matrix controlled-release device.

In another embodiment, the CETP inhibitor is in the form of an osmotic controlled-release device. The osmotic controlled-release device comprises (1) a core comprising the CETP inhibitor in solubility-improved form and an osmotic agent, and (2) a non-dissolving, non-eroding coating surrounding said core. The HMG-CoA reductase inhibitor is in the form of an immediate release coating or layer around the osmotic controlled-release device. An immediate release portion of the CETP inhibitor can also be included in the immediate release coating or layer around the osmotic controlled-release device.

In yet another embodiment, the dosage form comprises a tri-layer tablet comprising (1) a composition comprising the CETP inhibitor; (2) a composition comprising the HMG-CoA reductase inhibitor, (3) a sweller-layer composition sandwiched between (1) and (2), and (4) a water permeable coating surrounding (1), (2), and (3), wherein (1) is designed for controlled release of the CETP inhibitor and (2) is designed for immediate release of the HMG-CoA reductase inhibitor and potentially a portion of the CETP inhibitor.

In yet another embodiment, the dosage form comprises a plurality of controlled-release multiparticulates or granules comprising the CETP inhibitor in solubility-improved form and a plurality of immediate-release multiparticulates or granules comprising the HMG-CoA reductase inhibitor. A portion of the CETP inhibitor may be included in the dosage form as immediate-release multiparticulate or granules.

In yet another embodiment, the dosage form comprises a capsule, the capsule comprising a controlled-release device comprising the CETP inhibitor, the device selected from the group consisting of a matrix controlled-release device, an osmotic controlled-release device, and controlled-release multiparticulates. The capsule further comprises an immediate-release composition comprising an HMG-CoA reductase inhibitor. The capsule also comprises a portion of the CETP inhibitor as an immediate-release composition.

In yet another embodiment, the dosage form comprises a kit comprising at least two separate compositions: (1) one containing a controlled-release device comprising the CETP inhibitor in solubility-improved form, and (2) one containing the HMG-CoA reductase inhibitor and the CETP inhibitor in immediate release form. The kit includes means for containing the separate compositions. Alternatively the kit can be comprised of at least two separate compositions: (1) one containing a controlled-release device comprising the CETP inhibitor in solubility-improved form and a portion of the CETP inhibitor in immediate release form, and (2) one containing the HMG-CoA reductase inhibitor in immediate release form. In another aspect the dosage form comprises a kit comprising at least three separate compositions: (1) one containing a controlled-release device comprising the CETP inhibitor in solubility-improved form, (2) one containing the CETP inhibitor in immediate-release form, and (3) one containing the HMG-CoA reductase inhibitor in immediate release form.

In another aspect, the dosage forms of the present invention may be used to treat any condition, which is subject to treatment by administering a CETP inhibitor and an HMG-CoA reductase inhibitor, as disclosed in commonly assigned, copending U.S. Patent Application No. 2002/0035125A1, the disclosure of which is herein incorporated by reference.

It is to be understood that both the foregoing general description and the following detailed description are exemplary and explanatory only and are not restrictive of the invention, as claimed.

### BRIEF DESCRIPTION OF THE DRAWINGS

FIGS. 1-7 are schematic drawings of cross sections of exemplary embodiments of dosage forms of the present invention.

### DETAILED DESCRIPTION OF THE PREFERRED EMBODIMENTS

The present invention may be understood more readily by reference to the following detailed description of exemplary embodiments of the invention and the examples included therein.

Before the present dosage forms and methods are disclosed and described, it is to be understood that this invention is not limited to specific synthetic methods of making that may of course vary. It is also to be understood that the terminology used herein is for the purpose of describing particular embodiments only and is not intended to be limiting.

By “immediate release” is meant broadly that the HMG-CoA reductase inhibitor, and the portion of the immediate release CETP inhibitor that is in an immediate release form is released such that at least about 70 wt % of the drug initially present in the dosage form is released within one hour or less following introduction to a use environment. Immediate release of the drugs may be accomplished by any means known in the pharmaceutical arts, including immediate release coatings, immediate release layers, and immediate release multiparticulates or granules. The portion of the CETP inhibitor that is immediate release should be no greater than about 50% of the entire amount of CETP inhibitor present in the dosage form.

By “controlled release” is meant broadly that the CETP inhibitor is released at a rate that is slower than immediate release. Specific embodiments can be in the form of a sustained release oral dosage form, or, alternatively, in the form of a delayed release dosage form, or alternatively, in the form of an oral dosage form which exhibits a combination of sustained and delayed release characteristics. The term “controlled” is generic to “sustained” and “delayed.” Thus, “controlled release” is intended to embrace sustained release and sustained release after a lag time of the CETP inhibitor. Sustained release characteristics include dosage forms that release the CETP inhibitor according to zero-order, first-order, mixed-order or other kinetics. Controlled release of the CETP inhibitor may be accomplished by any means known in the pharmaceutical arts, including use of matrix controlled-release devices, osmotic controlled-release devices, and multiparticulate controlled-release devices. Devices for controlled release of CETP inhibitors are disclosed in further detail in commonly assigned, co-pending U.S. patent application Ser. No. 10/349,600, filed Jan. 23, 2003, entitled “Controlled Release Pharmaceutical Dosage Forms of a Cholesteryl Ester Transfer Protein Inhibitor,” the disclosures of which are hereby incorporated by reference.

Reference to a “use environment” can either mean in vivo fluids, such as the GI tract, subdermal, intranasal, buccal, intrathecal, ocular, intraaural, subcutaneous spaces, vaginal tract, arterial and venous blood vessels, pulmonary tract or intramuscular tissue of an animal, such as a mammal and particularly a human, or the in vitro environment of a test solution, such as phosphate buffered saline (PBS), simulated intestinal buffer without enzymes (SIN), or a Model Fasted Duodenal (MFD) solution. An appropriate PBS solution is an aqueous solution comprising 20 mM sodium phosphate (Na₂HPO₄), 47 mM potassium phosphate (KH₂PO₄), 87 mM NaCl, and 0.2 mM KCl, adjusted to pH 6.5 with NaOH. An appropriate SIN solution is 50 mM KH₂PO₄ adjusted to pH 7.4. An appropriate MFD solution is the same PBS solution wherein additionally is present 7.3 mM sodium taurocholic acid and 1.4 mM of 1-palmitoyl-2-oleyl-sn-glycero-3-phosphocholine.

“Administration” to a use environment means, where the in vivo use environment is the GI tract, delivery by ingestion or swallowing or other such means to deliver the drugs. One skilled in the art will understand that “administration” to other in vivo use environments means contacting the use environment with the composition of the invention using methods known in the art. See for example, Remington: The Science and Practice of Pharmacy, 20^{th} Edition (2000). Where the use environment is in vitro, “administration” refers to placement or delivery of the dosage form to the in vitro test medium.

Release rates, suitable dosage forms, CETP inhibitors, solubility-improved forms, and HMG-CoA reductase inhibitors are discussed in more detail below.

### Release Rates

The dosage forms of the present invention provide (1) immediate-release of an HMG-CoA reductase inhibitor and (2) controlled-release of a CETP inhibitor in a solubility-improved form and (3) immediate release of a CETP inhibitor in a solubility-improved form. As used herein, the rate of release of an immediate release drug from a dosage form is characterized by the percentage of the drug initially present in the dosage form that is released at one hour after administering the dosage form to a use environment. A dosage form is within the scope of the present invention if at one hour after administering the dosage form to a use environment, the dosage form has released at least about 70 wt % of each immediate release drug initially present in the dosage form. Preferably, the dosage form has released at least about 80 wt % at one hour, and more preferably, at least about 90 wt % at one hour after administering the dosage form to a use environment.

The dosage form of the present invention provides controlled release of the CETP inhibitor, meaning that the dosage form releases the controlled release portion of the CETP inhibitor at an average rate that is slower than immediate release. The release of CETP inhibitor from the dosage forms of the present invention may be characterized in terms of the time duration between introducing the dosage form to an environment of use and the time at which less than about 70% of the CETP inhibitor has left the dosage form. Description of the CETP inhibitor release rate is complicated by the fact that such dosage forms have a portion of the CETP inhibitor as immediate release, and may release the CETP inhibitor according to zero-order, first-order, mixed-order or other kinetics. To avoid confusion, we describe release rates in terms of the time duration between dosing the dosage form to an environment of use and the time at which less than about 70% of the CETP inhibitor has left the dosage form. This description applies to all dosage forms that release CETP inhibitor, regardless of the shape of the percent released vs. time curve and is intended to embrace sustained release dosage forms as well as dosage forms that exhibit sustained release after an initial lag time and dosage forms that exhibit sustained release after an initial immediate release. Thus, by “controlled release” of a CETP inhibitor is meant a dosage form that releases less than about 70 wt % of the CETP inhibitor initially present in the dosage form at 1 hour following introduction to a use environment. By “sustained release” is meant a dosage form wherein the CETP inhibitor is released slowly over time after administration to the use environment. Thus, the time to release less than about 70 wt % of the CETP inhibitor initially present in the dosage form is greater than about 1 hour. In one embodiment, the time to release less than about 70% of the CETP inhibitor initially present in the dosage form is at least about 2 hours, preferably at least about 3 hours, more preferably at least about 4 hours.

However, the release of CETP inhibitor from the dosage form should not be too slow. Thus, it is also preferred that the time to release about 70% of the CETP inhibitor initially present in the dosage form be about 24 hours or less, more preferably about 20 hours or less, and most preferably about 18 hours or less.

A dosage form is within the scope of the present invention if at one hour after administering the dosage form to a use environment, the dosage form has released at least about 70 wt % of the portion of the immediate release CETP inhibitor initially present in the dosage form. Preferably, the dosage form has released at least about 80 wt % of the portion of the immediate release CETP inhibitor initially present at one hour, and more preferably, at least about 90 wt % of the portion of the immediate release CETP inhibitor initially present at one hour after administering the dosage form to a use environment.

The controlled release of CETP inhibitor from the dosage form may also be characterized by an average rate of release of CETP inhibitor per hour for a time period, defined as the wt % of CETP inhibitor present in the dosage form released during the time period divided by the duration (in hours) of the time period. For example, if the dosage form releases 70 wt % of the CETP inhibitor initially present in the dosage form after 16 hours, the average rate of release of CETP inhibitor is 4.4 wt %/hour (70 wt %/16 hours). While the average rate of release may be calculated at any time period following introduction to the use environment, conventionally the time used is the time required to release 70 wt % of the CETP inhibitor initially present in the dosage form.

Thus, the inventive dosage forms have an average rate of release of the controlled release CETP inhibitor of less than about 70 wt %/hour. Preferably, the controlled release dosage forms of the present invention release CETP inhibitor at an average rate that is about 35 wt %/hour or less, more preferably about 23 wt %/hour or less, and even more preferably about 17.5 wt %/hour or less. It is also preferred that the controlled release dosage forms of the present invention release CETP inhibitor at an average rate that is about 2.9 wt %/hour or more, preferably about 3.5 wt %/hour or more, more preferably about 3.9 wt %/hour or more.

The dosage form of the present invention provides controlled release of the CETP inhibitor relative to an immediate release dosage form control consisting of an equivalent amount of the CETP inhibitor in the same solubility-improved form dosed as an oral powder for constitution. In one embodiment, when the use environment is the GI tract of a mammal, the dosage form provides a time to reach maximum drug concentration (Tₘₐₓ) in the blood of the mammal following administration that is longer than the immediate release dosage form control. Preferably, the Tₘₐₓ in the blood is at least about 1.25-fold longer than the immediate release dosage form control, preferably at least about 1.5-fold longer, and more preferably at least about 2-fold longer. In addition, the maximum concentration of drug (Cₘₐₓ) in the blood is less than or equal to about 80%, and may be less than or equal to about 65%, or even less than or equal to about 50% of the Cₘₐₓ provided by the immediate release dosage form control. Both Tₘₐₓ and Cₘₐₓ may be compared in either the fed or fasted state, and the dosage form meets the above criteria for at least one of, and preferably both, the fed and fasted state.

Depending on the portion of the CETP inhibitor that is provided as immediate release, the dosage form will provide a similar time to reach maximum drug concentration (Tₘₐₓ) in the blood of the mammal relative to an equivalent amount of the CETP inhibitor in the same solubility-improved form dosed as an oral powder for constitution.

It will be understood that the inclusion of an immediate release portion of CETP inhibitor to the dosage form will provide a similar time to reach maximum drug concentration (Tₘₐₓ) in the blood of the mammal relative to an equivalent amount of the CETP inhibitor in the same solubility-improved form dosed as an oral powder for constitution.

In another aspect, the dosage forms of the present invention provide controlled release of the CETP inhibitor which, after oral dosing, elicit one or more of the following effects: (a) about 50% or more, preferably about 70% or more, more preferably about 80% or more, even more preferably about 90% or more inhibition of plasma CETP, for about 12 hours or more, preferably about 16 hours or more; more preferably about 24 hours or more; (b) a decrease of 20% or more in mean plasma Cₘₐₓ relative to a dosage form that provides immediate release of the same amount of the solubility-improved form of the CETP inhibitor; (c) a mean increase in HDL cholesterol level of about 20% or greater, after dosing for 8 weeks; and (d) a mean decrease in LDL cholesterol levels of about 10% or greater, after dosing for 8 weeks. In other words, the dosage form, following administration to an in vivo use environment, provides at least one of: (i) at least about 50% inhibition of plasma cholesteryl ester transfer protein for at least about 12 hours; (ii) a maximum drug concentration in the blood that is less than or equal to about 80% of the maximum drug concentration in the blood provided by a dosage form that provides immediate release of the same amount of the solubility-improved form of said CETP inhibitor; (iii) a mean HDL cholesterol level after dosing for 8 weeks that is at least about 1.2-fold that obtained prior to dosing; and (iv) a mean LDL cholesterol level after dosing for 8 weeks that is less than or equal to about 90% that obtained prior to dosing.

Preferred embodiments exhibit two of the above effects. More preferred embodiments exhibit three or four of the above effects.

The dosage forms of the present invention may be dosed to a human subject in the fasted or fed state. It is preferred that they be dosed in the fed state.

Preferred CETP inhibitor doses and CETP inhibitor release rates from the dosage forms of this invention may be determined by pharmacokinetic (PK) modeling for individual CETP inhibitors, or by clinical experimentation (i.e. in human subjects or patients) as familiar to those experienced in the art. PK modeling may also be used to predict Cₘₐₓ for various CETP inhibitor doses and release rates, in order to identify those doses and release rates that will decrease Cₘₐₓ by 20% or more, relative to an immediate release dosage form at the same dose.

In one aspect, when the CETP inhibitor is [2R,4S] 4-[(3,5-bis-trifluoromethyl-benzyl)-methoxycarbonyl-amino]-2-ethyl-6-trifluoromethyl-3,4-dihydro-2H-quinoline-1-carboxylic acid ethyl ester (also known as torcetrapib), the dosage forms of the present invention, after oral dosing, elicit one or more of the following effects: (a) plasma concentrations of torcetrapib which exceed about 70 ng/mL, preferably about 110 ng/mL, more preferably about 160 ng/mL, even more preferably about 325 ng/mL for a period of around 12 hour or greater, preferably 16 hour or greater, more preferably about 24 hours or greater; (b) about 50% or more, preferably about 70% or more, more preferably about 80% or more, even more preferably about 90% or more inhibition of plasma CETP, for about 12 hours or more, preferably about 16 hours or more, more preferably about 24 hours or more; and (c) a decrease of 20% or more in mean plasma Cₘₐₓ relative to a dosage form that provides immediate release of the same amount of the solubility-improved form of torcetrapib; (d) a mean increase in HDL cholesterol level of about 20% or greater, after dosing for 8 weeks; and (e) a mean decrease in LDL cholesterol levels of about 10% or greater, after dosing for 8 weeks.

Preferred embodiments exhibit two of the above effects. More preferred embodiments exhibit three or more of the above effects.

The dosage forms of the present invention comprising torcetrapib may be dosed to a human subject in the fasted or fed state. It is preferred that they be dosed in the fed state. The dosage forms of the present invention are dosed at most twice daily (“BID”), preferably once daily (“QD”). The achievement of this aspect depends upon the CETP inhibitor dose and the CETP inhibitor release rate from the dosage form.

Details of the desired release profiles for CETP inhibitors are disclosed in further detail in commonly assigned, co-pending U.S. patent application Ser. No. 10/349,600, filed Jan. 23, 2003, entitled “Controlled Release Pharmaceutical Dosage Forms of a Cholesteryl Ester Transfer Protein Inhibitor,” the disclosures of which are hereby incorporated by reference.

An in vitro test may be used to determine whether a dosage form provides a release profile within the scope of the present invention. In vitro tests are well known in the art. The in vitro tests are designed to mimic the behavior of the dosage form in vivo. One example is a so-called “direct” test, where the dosage form is placed into a stirred USP type 2 dissolution flask containing 900 mL of a dissolution medium maintained at 37° C., such as a buffer solution simulating a gastric environment (10 mM HCl, 100 mM NaCl, pH 2.0, 261 mOsm/kg) or the PBS or MFD solutions previously described. One skilled in the art will understand that in such tests the dissolution medium need not act as a sink for the drug in the dosage form. This is particularly true of osmotic dosage forms where the rate at which undissolved drug extrudes from the osmotic dosage form is not substantially affected by the solubility of the drug in the dissolution medium. However, for dosage forms that deliver the drug in the dissolved state, it is preferred that a dissolution medium be chosen in which the solubility of the drug in the medium times the volume of the media exceeds the total mass of drug dosed; that is, the media should act as a sink for the drug. By “sink” is meant that the composition and volume of the dissolution medium is sufficient such that a quantity of drug alone equivalent to that in the dosage form will dissolve into the dissolution medium. Preferably, the composition and volume of dissolution medium is sufficient that a quantity of drug equivalent to at least about 2-fold that in the dosage form will dissolve in the dissolution medium. In most cases the CETP inhibitor is sufficiently insoluble in aqueous media that a surfactant, such as sodium lauryl sulfate, cetyltrimethylammonium bromide (CTAB), cetyltrimethylammonium chloride (CTAC), or other excipients may be added to the dissolution medium to raise the solubility of the drug and ensure the dissolution medium acts as a sink for the drug(s). The dosage form is placed in the dissolution medium, and the medium is stirred using paddles that rotate at a rate of 50 rpm. When the dosage form is in the form of a tablet, capsule or other solid dosage form, the dosage form may be placed in a wire support to keep the dosage form off of the bottom of the flask, so that all of its surfaces are exposed to the dissolution media. Alternatively, dissolution is performed in USP apparatus 1 placing the dosage form in baskets with or without sinkers rotating at 100 rpm or faster. Samples of the dissolution medium are taken at periodic intervals using a VanKel VK8000 autosampling dissoette with automatic receptor solution replacement. The concentration of dissolved drug in the dissolution medium is then determined by High Performance Liquid Chromatography (HPLC), by comparing UV absorbance of samples to the absorbance of drug standards. The mass of dissolved drug in the dissolution medium is then calculated from the concentration of drug in the medium and the volume of the medium, which value is used to calculate the actual amount of drug released from the dosage form, taking into consideration the mass of drug originally present in the dosage form.

The dosage forms of the present invention may also be evaluated using a “residual test,” which is performed as follows. A plurality of dosage forms are each placed into separate stirred USP type 2 dissoette flasks containing 900 mL of a buffer solution at 37° C. simulating a gastric or intestinal environment. After a given time interval, a dosage form is removed from a flask, released material is removed from the surface of the dosage form, and the dosage form cut in half and placed in 150 mL of a recovery solution as follows. For the first two hours, the dosage form is stirred in 25 mL acetone or other solvent suitable to dissolve any coating on the dosage form. Next, 125 mL of methanol is added and stirring continued overnight at ambient temperature to dissolve the drug remaining in the dosage form. Approximately 2 mL of the recovery solution is removed and centrifuged, and 250 μL of supernatant added to an HPLC vial and diluted with 750 μL methanol. Residual drug is then analyzed by HPLC. The amount of drug remaining in the dosage form is subtracted from the total drug initially present in the dosage form to obtain the amount released at each time interval.

Alternatively, an in vivo test may be used to determine whether a dosage form provides a drug release profile within the scope of the present invention. However, due to the inherent difficulties and complexity of the in vivo procedure, it is preferred that in vitro procedures be used to evaluate dosage forms even though the ultimate use environment is often the human GI tract. The in vitro tests described above are expected to approximate in vivo behavior, and a dosage form that meets the in vitro release rates described herein are within the scope of the invention. Dosage forms are dosed to a group of test subjects, such as humans, and drug release and drug absorption is monitored either by (1) periodically withdrawing blood and measuring the serum or plasma concentration of drug or (2) measuring the amount of drug remaining in the dosage form following its exit from the anus (residual drug) or (3) both (1) and (2). In the second method, residual drug is measured by recovering the dosage form upon exit from the anus of the test subject and measuring the amount of drug remaining in the dosage form using the same procedure described above for the in vitro residual test. The difference between the amount of drug in the original dosage form and the amount of residual drug is a measure of the amount of drug released during the mouth-to-anus transit time. This test has limited utility since it provides only a single drug release time point but is useful in demonstrating the correlation between in vitro and in vivo release.

In one in vivo method of monitoring drug release and absorption, the serum or plasma drug concentration is plotted along the ordinate (y-axis) against the blood sample time along the abscissa α-axis). The data may then be analyzed to determine drug release rates using any conventional analysis, such as the Wagner-Nelson or Loo-Riegelman analysis. See also Welling, “Pharmacokinetics: Processes and Mathematics” (ACS Monograph 185, Amer. Chem. Soc., Washington, D.C., 1986). Treatment of the data in this manner yields an apparent in vivo drug release profile.

### Dosage Forms

The dosage forms of the present invention provide controlled-release of a CETP inhibitor in solubility-improved form, immediate-release of an HMG-CoA reductase inhibitor, and immediate release of a CETP inhibitor in a solubility improved form. Controlled-release of a CETP inhibitor is desirable for several reasons. It is often desirable to have a method of lowering the maximum CETP inhibitor concentration in the plasma (Cₘₐₓ) after dosing while still providing good bioavailability, in order to decrease undesirable side effects, relative to an immediate release dosage form containing an equivalent amount of CETP inhibitor. Furthermore, it is important that dosing of the CETP inhibitor be convenient, i.e. once-per-day (QD) or twice-per-day (BID), because patients who take multiple drugs may have a difficult time keeping track of which drugs to take at which time of day. Furthermore, some drugs such as CETP inhibitors are advantageously taken with a meal, and it is preferable to minimize the number of times per day that the drug is taken, to simplify the requirement that the drug be taken with a meal.

The means for providing controlled release of the CETP inhibitor in solubility-improved form can be any device or collection of devices known in the pharmaceutical arts that allow delivery of a drug in a controlled manner. The controlled-release means slowly releases the solubility-improved form of the CETP inhibitor to the use environment. The CETP inhibitor in solubility-improved form may be delivered into the use environment as a suspension, that is, as a plurality of small particles, the small particles comprising the controlled-release means, which allow the drug to dissolve at a controlled rate in the use environment. Exemplary controlled-release means include matrix controlled-release devices, osmotic controlled-release devices, and multiparticulate controlled-release devices. The controlled-release devices themselves may or may not dissolve.

Immediate release of an HMG-CoA reductase inhibitor is also desirable. The half life of many HMG-CoA reductase inhibitors is on the order of 20 hours or more. Immediate release of the HMG-CoA reductase inhibitor may be accomplished by any means known in the pharmaceutical arts. Exemplary methods include immediate release coatings, immediate release layers, immediate release multiparticulates or granules, and immediate release tablets, capsules, or pills. The immediate release composition may include the HMG-CoA reductase inhibitor alone or in combination with an immediate release portion of the CETP inhibitor mixed with excipients or other materials to aid in formation of the dosage form.

The present invention embraces any dosage form that combines a controlled-release means for the CETP inhibitor with an immediate release means for the HMG-CoA reductase inhibitor and an immediate release means for the CETP inhibitor. Such means can be combined as required to achieve the desired release profiles disclosed herein. Controlled-release means, immediate release means, and exemplary dosage forms of the present invention are discussed below.

### Controlled-Release Means

The means for providing controlled release of the CETP inhibitor in solubility-improved form can be any device or collection of devices known in the pharmaceutical arts that allow delivery of a drug in a controlled manner. Exemplary devices include erodible and non-erodible matrix controlled-release devices, osmotic controlled-release devices, and multiparticulate controlled-release devices.

### Matrix Controlled Release Devices

In one embodiment, the CETP inhibitor in solubility-improved form is incorporated into an erodible or non-erodible polymeric matrix controlled release device. By an erodible matrix is meant aqueous-erodible or water-swellable or aqueous-soluble in the sense of being either erodible or swellable or dissolvable in pure water or requiring the presence of an acid or base to ionize the polymeric matrix sufficiently to cause erosion or dissolution. When contacted with the aqueous environment of use, the erodible polymeric matrix imbibes water and forms an aqueous-swollen gel or “matrix” that entraps the solubility-improved form of the CETP inhibitor. The aqueous-swollen matrix gradually erodes, swells, disintegrates or dissolves in the environment of use, thereby controlling the release of the CETP inhibitor to the environment of use. Examples of such devices are disclosed more fully in commonly assigned pending U.S. patent application Ser. No. 09/495,059 filed Jan. 31, 2000 which claimed the benefit of priority of provisional patent application Ser. No. 60/119,400 filed Feb. 10, 1999, the relevant disclosure of which is herein incorporated by reference.

The erodible polymeric matrix into which the CETP inhibitor in solubility-improved form is incorporated may generally be described as a set of excipients that are mixed with the solubility-improved form following its formation that, when contacted with the aqueous environment of use imbibes water and forms a water-swollen gel or “matrix” that entraps the drug form. Drug release may occur by a variety of mechanisms: the matrix may disintegrate or dissolve from around particles or granules of the drug in solubility-improved form; or the drug may dissolve in the imbibed aqueous solution and diffuse from the tablet, beads or granules of the device. A key ingredient of this water-swollen matrix is the water-swellable, erodible, or soluble polymer, which may generally be described as an osmopolymer, hydrogel or water-swellable polymer. Such polymers may be linear, branched, or crosslinked. They may be homopolymers or copolymers. Although they may be synthetic polymers derived from vinyl, acrylate, methacrylate, urethane, ester and oxide monomers, they are most preferably derivatives of naturally occurring polymers such as polysaccharides or proteins.

Such materials include naturally occurring polysaccharides such as chitin, chitosan, dextran and pullulan; gum agar, gum arabic, gum karaya, locust bean gum, gum tragacanth, carrageenans, gum ghatti, guar gum, xanthan gum and scleroglucan; starches such as dextrin and maltodextrin; hydrophilic colloids such as pectin; phosphatides such as lecithin; alginates such as ammonium alginate, sodium, potassium or calcium alginate, propylene glycol alginate; gelatin; collagen; and cellulosics. By “cellulosics” is meant a cellulose polymer that has been modified by reaction of at least a portion of the hydroxyl groups on the saccharide repeat units with a compound to form an ester-linked or an ether-linked substituent. For example, the cellulosic ethyl cellulose has an ether linked ethyl substituent attached to the saccharide repeat unit, while the cellulosic cellulose acetate has an ester linked acetate substituent.

A preferred class of cellulosics for the erodible matrix comprises aqueous-soluble and aqueous-erodible cellulosics such as ethyl cellulose (EC), methylethyl cellulose (MEC), carboxymethyl cellulose (CMC), carboxymethyl ethylcellulose (CMEC), hydroxyethyl cellulose (HEC), hydroxypropyl cellulose (HPC), cellulose acetate (CA), cellulose propionate (CP), cellulose butyrate (CB), cellulose acetate butyrate (CAB), cellulose acetate phthalate (CAP), cellulose acetate trimellitate (CAT), hydroxypropyl methyl cellulose (HPMC), hydroxypropyl methyl cellulose phthalate (HPMCP), hydroxypropyl methyl cellulose acetate succinate (HPMCAS), hydroxypropyl methyl cellulose acetate trimellitate (HPMCAT), and ethylhydroxy ethylcellulose (EHEC). A particularly preferred class of such cellulosics comprises various grades of low viscosity (MW less than or equal to 50,000 daltons) and high viscosity (MW greater than 50,000 daltons) HPMC. Commercially available low viscosity HPMC polymers include the Dow METHOCEL series E5, E15LV, E50LV and K100LV, while high viscosity HPMC polymers include E4MCR, E10MCR, K₄M, K15M and K100M; especially preferred in this group are the METHOCEL (Trademark) K series. Other commercially available types of HPMC include the Shin Etsu METOLOSE 90SH series.

Although the primary role of the erodible matrix material is to control the rate of release of CETP inhibitor in solubility-improved form to the environment of use, the inventors have found that the choice of matrix material can have a large effect on the maximum drug concentration attained by the device as well as the maintenance of a high drug concentration. In one embodiment, the matrix material is a concentration-enhancing polymer, as defined herein below.

Other materials useful as the erodible matrix material include, but are not limited to, pullulan, polyvinyl pyrrolidone, polyvinyl alcohol, polyvinyl acetate, glycerol fatty acid esters, polyacrylamide, polyacrylic acid, copolymers of ethacrylic acid or methacrylic acid (EUDRAGIT®), Rohm America, Inc., Piscataway, N.J.) and other acrylic acid derivatives such as homopolymers and copolymers of butylmethacrylate, methylmethacrylate, ethylmethacrylate, ethylacrylate, (2-dimethylaminoethyl)methacrylate, and (trimethylaminoethyl)methacrylate chloride.

The erodible matrix polymer may contain a wide variety of the same types of additives and excipients known in the pharmaceutical arts, including osmopolymers, osmagens, solubility-enhancing or -retarding agents and excipients that promote stability or processing of the device.

Alternatively, the compositions of the present invention may be administered by or incorporated into a non-erodible matrix device. In such devices, the CETP inhibitor in solubility-improved form is distributed in an inert matrix. The drug is released by diffusion through the inert matrix. Examples of materials suitable for the inert matrix include insoluble plastics, such as methyl acrylate-methyl methacrylate copolymers, polyvinyl chloride, and polyethylene; hydrophilic polymers, such as ethyl cellulose, cellulose acetate, and crosslinked polyvinylpyrrolidone (also known as crospovidone); and fatty compounds, such as carnauba wax, microcrystalline wax, and triglycerides. Such devices are described further in Remington: The Science and Practice of Pharmacy, 20^{th} edition (2000).

Matrix controlled release devices may be prepared by blending the CETP inhibitor in solubility-improved form and other excipients together, and then forming the blend into a tablet, caplet, pill, or other device formed by compressive forces. Such compressed devices may be formed using any of a wide variety of presses used in the fabrication of pharmaceutical devices. Examples include single-punch presses, rotary tablet presses, and multilayer rotary tablet presses, all well known in the art. See for example, Remington: The Science and Practice of Pharmacy, 20^{th} Edition, 2000. The compressed device may be of any shape, including round, oval, oblong, cylindrical, or triangular. The upper and lower surfaces of the compressed device may be flat, round, concave, or convex.

When formed by compression, the device preferably has a “strength” of at least about 5 kiloponds (kp), and more preferably at least about 7 kp. Here, “strength” is the fracture force, also known as the tablet “hardness,” required to fracture a tablet formed from the materials, divided by the maximum cross-sectional area of the tablet normal to that force. The fracture force may be measured using a Schleuniger Tablet Hardness Tester, Model 6D. The compression force required to achieve this strength will depend on the size of the tablet, but generally will be greater than about 5 kN. Friability is a well-known measure of a device's resistance to surface abrasion that measures weight loss in percentage after subjecting the device to a standardized agitation procedure. Friability values of from 0.8 to 1.0% are regarded as constituting the upper limit of acceptability. Devices having a strength of greater than 5 kp generally are very robust, having a friability of less than 0.5%,

Other methods for forming matrix controlled-release devices are well known in the pharmaceutical arts. See for example, Remington: The Science and Practice of Pharmacy, 20^{th} Edition, 2000.

### Osmotic Controlled Release Devices

Alternatively, the CETP inhibitor in solubility-improved form may be incorporated into an osmotic controlled release device. Such devices have at least two components: (a) the core which contains an osmotic agent and the solubility-improved form of the CETP inhibitor; and (b) a water permeable, non-dissolving and non-eroding coating surrounding the core, the coating controlling the influx of water to the core from an aqueous environment of use so as to cause drug release by extrusion of some or all of the core to the environment of use. The osmotic agent contained in the core of this device may be an aqueous-swellable hydrophilic polymer or it may be an osmogen, also known as an osmagent. The coating is preferably polymeric, aqueous-permeable, and has at least one delivery port. Examples of such devices are disclosed more fully in commonly assigned pending U.S. patent application Ser. No. 09/495,061 filed Jan. 31, 2000 which claimed the benefit of priority of provisional Patent Application Ser. No. 60/119,406 filed Feb. 10, 1999, and U.S. patent application Ser. No. 10/352,283, filed Jan. 27, 2003, which claimed the benefit of priority of provisional Patent Application Ser. No. 60/353,151 filed Feb. 1, 2002, the disclosures of which are herein incorporated by reference.

In addition to the solubility-improved form of the CETP inhibitor, the core of the osmotic device optionally includes an “osmotic agent.” By “osmotic agent” is meant any agent that creates a driving force for transport of water from the environment of use into the core of the device. Exemplary osmotic agents are water-swellable hydrophilic polymers, and osmogens (or osmagents). Thus, the core may include water-swellable hydrophilic polymers, both ionic and nonionic, often referred to as “osmopolymers” and “hydrogels.” The amount of water-swellable hydrophilic polymers present in the core may range from about 5 to about 80 wt %, preferably 10 to 50 wt %. Exemplary materials include hydrophilic vinyl and acrylic polymers, polysaccharides such as calcium alginate, polyethylene oxide (PEO), polyethylene glycol (PEG), polypropylene glycol (PPG), poly(2-hydroxyethyl methacrylate), poly(acrylic) acid, poly(methacrylic) acid, polyvinylpyrrolidone (PVP) and crosslinked PVP, polyvinyl alcohol (PVA), PVA/PVP copolymers and PVA/PVP copolymers with hydrophobic monomers such as methyl methacrylate, vinyl acetate, and the like, hydrophilic polyurethanes containing large PEO blocks, sodium croscarmellose, carrageenan, hydroxyethyl cellulose (HEC), hydroxypropyl cellulose (HPC), hydroxypropyl methyl cellulose (HPMC), carboxymethyl cellulose (CMC) and carboxyethyl cellulose (CEC), sodium alginate, polycarbophil, gelatin, xanthan gum, and sodium starch glycolate. Other materials include hydrogels comprising interpenetrating networks of polymers that may be formed by addition or by condensation polymerization, the components of which may comprise hydrophilic and hydrophobic monomers such as those just mentioned. Preferred polymers for use as the water-swellable hydrophilic polymers include PEO, PEG, PVP, sodium croscarmellose, HPMC, sodium starch glycolate, polyacrylic acid and crosslinked versions or mixtures thereof.

The core may also include an osmogen (or osmagent). The amount of osmogen present in the core may range from about 2 to about 70 wt %, preferably 10 to 50 wt %. Typical classes of suitable osmogens are water-soluble organic acids, salts and sugars that are capable of imbibing water to thereby effect an osmotic pressure gradient across the barrier of the surrounding coating. Typical useful osmogens include magnesium sulfate, magnesium chloride, calcium chloride, sodium chloride, lithium chloride, potassium sulfate, sodium carbonate, sodium sulfite, lithium sulfate, potassium chloride, sodium sulfate, mannitol, xylitol, urea, sorbitol, inositol, raffinose, sucrose, glucose, fructose, lactose, citric acid, succinic acid, tartaric acid, and mixtures thereof. Particularly preferred osmogens are glucose, lactose, sucrose, mannitol, xylitol and sodium chloride.

The core may include a wide variety of additives and excipients that enhance the performance of the dosage form or that promote stability, tableting or processing. Such additives and excipients include tableting aids, surfactants, water-soluble polymers, pH modifiers, fillers, binders, pigments, disintegrants, antioxidants, lubricants and flavorants. Exemplary of such components are microcrystalline cellulose; metallic salts of acids such as aluminum stearate, calcium stearate, magnesium stearate, sodium stearate, and zinc stearate; pH control agents such as buffers, organic acids and organic acid salts and organic and inorganic bases; fatty acids, hydrocarbons and fatty alcohols such as stearic acid, palmitic acid, liquid paraffin, stearyl alcohol, and palmitol; fatty acid esters such as glyceryl (mono- and di-)stearates, triglycerides, glyceryl(palmiticstearic) ester, sorbitan esters, such as sorbitan monostearate, saccharose monostearate, saccharose monopalmitate, and sodium stearyl fumarate; polyoxyethylene sorbitan esters; surfactants, such as alkyl sulfates such as sodium lauryl sulfate and magnesium lauryl sulfate; polymers such as polyethylene glycols, polyoxyethylene glycols, polyoxyethylene and polyoxypropylene ethers and their copolymers, and polytetrafluoroethylene; and inorganic materials such as talc and dicalcium phosphate; cyclodextrins; sugars such as lactose and xylitol; and sodium starch glycolate. Examples of disintegrants are sodium starch glycolate (e.g., Explotab™), microcrystalline cellulose (e.g., Avicel™), microcrystalline silicified cellulose (e.g., ProSolv™), croscarmellose sodium (e.g., Ac-Di-Sol™).

When the solubility-improved form is a solid amorphous dispersion formed by a solvent process, such additives may be added directly to the spray-drying solution when forming the CETP inhibitor/concentration-enhancing polymer dispersion such that the additive is dissolved or suspended in the solution as a slurry. Alternatively, such additives may be added following the spray-drying process to aid in forming the final controlled release device. Such solubility-enhancing and other additives may also be blended with other solubility-improved forms of the CETP inhibitor.

One embodiment of an osmotic device consists of one or more drug layers containing the solubility-improved form of the CETP inhibitor, such as a solid amorphous drug/polymer dispersion, and a sweller layer that comprises a water-swellable polymer, with a coating surrounding the drug layer and sweller layer. Each layer may contain other excipients such as tableting aids, osmagents, surfactants, water-soluble polymers and water-swellable polymers.

Such osmotic delivery devices may be fabricated in various geometries including bilayer, wherein the core comprises a drug layer and a sweller layer adjacent to each other; trilayer, wherein the core comprises a sweller layer “sandwiched” between two drug layers; and concentric, wherein the core comprises a central sweller composition surrounded by the drug layer.

The coating of such a tablet comprises a membrane permeable to water but substantially impermeable to drug and excipients contained within. The coating contains one or more exit passageways or ports in communication with the drug-containing layer(s) for delivering the drug composition. The drug-containing layer(s) of the core contains the drug composition (including optional osmagents and hydrophilic water-soluble polymers), while the sweller layer consists of an expandable hydrogel, with or without additional osmotic agents.

When placed in an aqueous medium, the tablet imbibes water through the membrane, causing the composition to form a dispensable aqueous composition, and causing the hydrogel layer to expand and push against the drug-containing composition, forcing the composition out of the exit passageway. The composition can swell, aiding in forcing the drug out of the passageway. Drug can be delivered from this type of delivery system either dissolved or dispersed in the composition that is expelled from the exit passageway.

The rate of drug delivery is controlled by such factors as the permeability and thickness of the coating, the osmotic pressure of the drug-containing layer, the degree of hydrophilicity of the hydrogel layer, and the surface area of the device. Those skilled in the art will appreciate that increasing the thickness of the coating will reduce the release rate, while any of the following will increase the release rate: increasing the permeability of the coating; increasing the hydrophilicity of the hydrogel layer; increasing the osmotic pressure of the drug-containing layer; or increasing the device's surface area.

Exemplary materials useful in forming the drug-containing composition, in addition to the solubility-improved form of the CETP inhibitor itself, include HPMC, PEO and PVP and other pharmaceutically acceptable carriers. In addition, osmagents such as sugars or salts, especially sucrose, lactose, xylitol, mannitol, or sodium chloride, may be added. Materials which are useful for forming the hydrogel layer include sodium CMC, PEO, poly(acrylic acid), sodium (polyacrylate), sodium croscarmellose, sodium starch glycolate, PVP, crosslinked PVP, and other high molecular weight hydrophilic materials. Particularly useful are PEO polymers having an average molecular weight from about 5,000,000 to about 7,500,000 daltons.

In the case of a bilayer geometry, the delivery port(s) or exit passageway(s) may be located on the side of the tablet containing the drug composition or may be on both sides of the tablet or even on the edge of the tablet so as to connect both the drug layer and the sweller layer with the exterior of the device. The exit passageway(s) may be produced by mechanical means or by laser drilling, or by creating a difficult-to-coat region on the tablet by use of special tooling during tablet compression or by other means.

The osmotic device can also be made with a homogeneous core surrounded by a semipermeable membrane coating, as in U.S. Pat. No. 3,845,770. The solubility-improved form of the CETP inhibitor can be incorporated into a tablet core and a semipermeable membrane coating can be applied via conventional tablet-coating techniques such as using a pan coater. A drug delivery passageway can then be formed in this coating by drilling a hole in the coating, either by use of a laser or mechanical means. Alternatively, the passageway may be formed by rupturing a portion of the coating or by creating a region on the tablet that is difficult to coat, as described above.

A particularly useful embodiment of an osmotic device comprises: (a) a single-layer compressed core comprising: (i) the solubility-improved form of the CETP inhibitor, (ii) a hydroxyethylcellulose, and (iii) an osmagent, wherein the hydroxyethylcellulose is present in the core from about 2.0% to about 35% by weight and the osmagent is present from about 15% to about 70% by weight; (b) a water-permeable layer surrounding the core; and (c) at least one passageway within the layer (b) for delivering the drug to a fluid environment surrounding the tablet. In a preferred embodiment, the device is shaped such that the surface area to volume ratio (of a water-swollen tablet) is greater than 0.6 mm⁻¹; more preferably greater than 1.0 mm⁻¹. It is preferred that the passageway connecting the core with the fluid environment be situated along the tablet band area. A particularly preferred shape is an oblong shape where the ratio of the tablet tooling axes, i.e., the major and minor axes which define the shape of the tablet, are between 1.3 and 3; more preferably between 1.5 and 2.5. In one embodiment, the combination of the solubility-improved form of the drug and the osmagent have an average ductility from about 100 to about 200 Mpa, an average tensile strength from about 0.8 to about 2.0 Mpa, and an average brittle fracture index less than about 0.2. The single-layer core may optionally include a disintegrant, a bioavailability enhancing additive, and/or a pharmaceutically acceptable excipient, carrier or diluent. Such devices are disclosed more fully in commonly owned, pending U.S. provisional Patent Application Ser. No. 60/353,151, entitled “Osmotic Delivery System,” the disclosure of which are incorporated herein by reference.

Entrainment of particles of the solubility-improved form of the CETP inhibitor in the extruding fluid during operation of such osmotic device is highly desirable. For the particles to be well entrained, the drug form is preferably well dispersed in the fluid before the particles have an opportunity to settle in the tablet core. One means of accomplishing this is by adding a disintegrant that serves to break up the compressed core into its particulate components. Examples of standard disintegrants include materials such as sodium starch glycolate (e.g., Explotab™), microcrystalline cellulose (e.g., Avicel™), microcrystalline silicified cellulose (e.g., ProSolv™) and croscarmellose sodium (e.g., Ac-Di-Sol™), and other disintegrants known to those skilled in the art. Depending upon the particular formulation, some disintegrants work better than others. Several disintegrants tend to form gels as they swell with water, thus hindering drug delivery from the device. Non-gelling, non-swelling disintegrants provide a more rapid dispersion of the drug particles within the core as water enters the core. Preferred non-gelling, non-swelling disintegrants are resins, preferably ion-exchange resins. A preferred resin is Amberlite™ IRP 88 (available from Rohm and Haas, Philadelphia, Pa.). When used, the disintegrant is present in amounts ranging from about 1-25% of the core composition.

Water-soluble polymers are added to keep particles of the solubility-improved drug form suspended inside the device before they can be delivered through the passageway(s) (e.g., an orifice). High viscosity polymers are useful in preventing settling. However, the polymer in combination with the drug is extruded through the passageway(s) under relatively low pressures. At a given extrusion pressure, the extrusion rate typically slows with increased viscosity. Certain polymers in combination with particles of the solubility-improved drug form high viscosity solutions with water but are still capable of being extruded from the tablets with a relatively low force. In contrast, polymers having a low weight-average, molecular weight (<about 300,000) do not form sufficiently viscous solutions inside the tablet core to allow complete delivery due to particle settling. Settling of the particles is a problem when such devices are prepared with no polymer added, which leads to poor drug delivery unless the tablet is constantly agitated to keep the particles from settling inside the core. Settling is also problematic when the particles are large and/or of high density such that the rate of settling increases.

Preferred water-soluble polymers for such osmotic devices do not interact with the drug. Non-ionic polymers are preferred. An example of a non-ionic polymer forming solutions having a high viscosity yet still extrudable at low pressures is Natrosol™ 250H (high molecular weight hydroxyethylcellulose, available from Hercules Incorporated, Aqualon Division, Wilmington, Del.; MW equal to about 1 million daltons and a degree of polymerization equal to about 3,700). Natrosol™ 250H provides effective drug delivery at concentrations as low as about 3% by weight of the core when combined with an osmagent. Natrosol™ 250H NF is a high-viscosity grade nonionic cellulose ether that is soluble in hot or cold water. The viscosity of a 1% solution of Natrosol™ 250H using a Brookfield LVT (30 rpm) at 25° C. is between about 1,500 and about 2,500 cps.

Preferred hydroxyethylcellulose polymers for use in these monolayer osmotic tablets have a weight-average, molecular weight from about 300,000 to about 1.5 million. The hydroxyethylcellulose polymer is typically present in the core in an amount from about 2.0% to about 35% by weight.

Another example of an osmotic device is an osmotic capsule. The capsule shell or portion of the capsule shell can be semipermeable. The capsule can be filled either by a powder or liquid consisting of the CETP inhibitor in solubility-improved form, excipients that imbibe water to provide osmotic potential, and/or a water-swellable polymer, or optionally solubilizing excipients. The capsule core can also be made such that it has a bilayer or multilayer composition analogous to the bilayer, trilayer or concentric geometries described above.

Another class of osmotic device useful in this invention comprises coated swellable tablets, as described in EP 378 404, incorporated herein by reference. Coated swellable tablets comprise a tablet core comprising the solubility-improved form of the drug and a swelling material, preferably a hydrophilic polymer, coated with a membrane, which contains holes, or pores through which, in the aqueous use environment, the hydrophilic polymer can extrude and carry out the drug composition. Alternatively, the membrane may contain polymeric or low molecular weight water-soluble “porosigens”. Porosigens dissolve in the aqueous use environment, providing pores through which the hydrophilic polymer and drug may extrude. Examples of porosigens are water-soluble polymers such as HPMC, PEG, and low molecular weight compounds such as glycerol, sucrose, glucose, and sodium chloride. In addition, pores may be formed in the coating by drilling holes in the coating using a laser or other mechanical means. In this class of osmotic devices, the membrane material may comprise any film-forming polymer, including polymers which are water permeable or impermeable, providing that the membrane deposited on the tablet core is porous or contains water-soluble porosigens or possesses a macroscopic hole for water ingress and drug release. Embodiments of this class of sustained release devices may also be multilayered, as described in EP 378 404 A2.

When the CETP inhibitor in solubility-improved form is a liquid or oil, such as a lipid vehicle formulation described herein, the osmotic controlled-release device may comprise a soft-gel or gelatin capsule formed with a composite wall and comprising the liquid formulation where the wall comprises a barrier layer formed over the external surface of the capsule, an expandable layer formed over the barrier layer, and a semipermeable layer formed over the expandable layer. A delivery port connects the liquid formulation with the aqueous use environment. Such devices are described more fully in U.S. Pat. Nos. 6,419,952, 6,342,249, 5,324,280, 4,672,850, 4,627,850, 4,203,440, and 3,995,631, all of which are incorporated herein by reference.

The osmotic controlled release devices of the present invention also comprise a coating. The essential constraints on the coating for an osmotic device are that it be water-permeable, have at least one port for the delivery of drug, and be non-dissolving and non-eroding during release of the drug formulation, such that drug is substantially entirely delivered through the delivery port(s) or pores as opposed to delivery primarily via permeation through the coating material itself. By “delivery port” is meant any passageway, opening or pore whether made mechanically, by laser drilling, by pore formation either during the coating process or in situ during use or by rupture during use. The coating should be present in an amount ranging from about 5 to 30 wt %, preferably 10 to 20 wt % relative to the core weight.

A preferred form of coating is a semipermeable polymeric membrane that has the port(s) formed therein either prior to or during use. Thickness of such a polymeric membrane may vary between about 20 and 800 μm, and is preferably in the range of 100 to 500 μm. The delivery port(s) should generally range in size from 0.1 to 3000 μm or greater, preferably on the order of 50 to 3000 μm in diameter. Such port(s) may be formed post-coating by mechanical or laser drilling or may be formed in situ by rupture of the coatings; such rupture may be controlled by intentionally incorporating a relatively small weak portion into the coating. Delivery ports may also be formed in situ by erosion of a plug of water-soluble material or by rupture of a thinner portion of the coating over an indentation in the core. In addition, delivery ports may be formed during coating, as in the case of asymmetric membrane coatings of the type disclosed in U.S. Pat. Nos. 5,612,059 and 5,698,220, the disclosures of which are incorporated by reference.

When the delivery port is formed in situ by rupture of the coating, a particularly preferred embodiment is a collection of beads that may be of essentially identical or of a variable composition. Drug is primarily released from such beads following rupture of the coating and, following rupture, such release may be gradual or relatively sudden. When the collection of beads has a variable composition, the composition may be chosen such that the beads rupture at various times following administration, resulting in the overall release of drug being sustained for a desired duration.

Coatings may be dense, microporous or “asymmetric,” having a dense region supported by a thick porous region such as those disclosed in U.S. Pat. Nos. 5,612,059 and 5,698,220. When the coating is dense the coating is composed of a water-permeable material. When the coating is porous, it may be composed of either a water-permeable or a water-impermeable material. When the coating is composed of a porous water-impermeable material, water permeates through the pores of the coating as either a liquid or a vapor.

Examples of osmotic devices that utilize dense coatings include U.S. Pat. Nos. 3,995,631 and 3,845,770, the disclosures of which pertaining to dense coatings are incorporated herein by reference. Such dense coatings are permeable to the external fluid such as water and may be composed of any of the materials mentioned in these patents as well as other water-permeable polymers known in the art.

The membranes may also be porous as disclosed in U.S. Pat. Nos. 5,654,005 and 5,458,887 or even be formed from water-resistant polymers. U.S. Pat. No. 5,120,548 describes another suitable process for forming coatings from a mixture of a water-insoluble polymer and a leachable water-soluble additive, the pertinent disclosures of which are incorporated herein by reference. The porous membranes may also be formed by the addition of pore-formers as disclosed in U.S. Pat. No. 4,612,008, the pertinent disclosures of which are incorporated herein by reference.

In addition, vapor-permeable coatings may even be formed from extremely hydrophobic materials such as polyethylene or polyvinylidene difluoride that, when dense, are essentially water-impermeable, as long as such coatings are porous.

Materials useful in forming the coating include various grades of acrylics, vinyls, ethers, polyamides, polyesters and cellulosic derivatives that are water-permeable and water-insoluble at physiologically relevant pHs, or are susceptible to being rendered water-insoluble by chemical alteration such as by crosslinking.

Specific examples of suitable polymers (or crosslinked versions) useful in forming the coating include plasticized, unplasticized and reinforced cellulose acetate (CA), cellulose diacetate, cellulose triacetate, CA propionate, cellulose nitrate, cellulose acetate butyrate (CAB), CA ethyl carbamate, CAP, CA methyl carbamate, CA succinate, cellulose acetate trimellitate (CAT), CA dimethylaminoacetate, CA ethyl carbonate, CA chloroacetate, CA ethyl oxalate, CA methyl sulfonate, CA butyl sulfonate, CA p-toluene sulfonate, agar acetate, amylose triacetate, beta glucan acetate, beta glucan triacetate, acetaldehyde dimethyl acetate, triacetate of locust bean gum, hydroxlated ethylene-vinylacetate, EC, PEG, PPG, PEG/PPG copolymers, PVP, HEC, HPC, CMC, CMEC, HPMC, HPMCP, HPMCAS, HPMCAT, poly(acrylic) acids and esters and poly-(methacrylic) acids and esters and copolymers thereof, starch, dextran, dextrin, chitosan, collagen, gelatin, polyalkenes, polyethers, polysulfones, polyethersulfones, polystyrenes, polyvinyl halides, polyvinyl esters and ethers, natural waxes and synthetic waxes.

A preferred coating composition comprises a cellulosic polymer, in particular cellulose ethers, cellulose esters and cellulose ester-ethers, i.e., cellulosic derivatives having a mixture of ester and ether substituents.

Another preferred class of coating materials are poly(acrylic) acids and esters, poly(methacrylic) acids and esters, and copolymers thereof.

A more preferred coating composition comprises cellulose acetate. An even more preferred coating comprises a cellulosic polymer and PEG. A most preferred coating comprises cellulose acetate and PEG.

Coating is conducted in conventional fashion, typically by dissolving or suspending the coating material in a solvent and then coating by dipping, spray coating, fluid bed coating or preferably by pan-coating. A preferred coating solution contains 5 to 15 wt % polymer. Typical solvents useful with the cellulosic polymers mentioned above include acetone, methyl acetate, ethyl acetate, isopropyl acetate, n-butyl acetate, methyl isobutyl ketone, methyl propyl ketone, ethylene glycol monoethyl ether, ethylene glycol monoethyl acetate, methylene dichloride, ethylene dichloride, propylene dichloride, nitroethane, nitropropane, tetrachloroethane, 1,4-dioxane, tetrahydrofuran, diglyme, water, and mixtures thereof. Pore-formers and non-solvents (such as water, glycerol and ethanol) or plasticizers (such as diethyl phthalate) may also be added in any amount as long as the polymer remains soluble at the spray temperature. Pore-formers and their use in fabricating coatings are described in U.S. Pat. No. 5,612,059, the pertinent disclosures of which are incorporated herein by reference.

Coatings may also be hydrophobic microporous layers wherein the pores are substantially filled with a gas and are not wetted by the aqueous medium but are permeable to water vapor, as disclosed in U.S. Pat. No. 5,798,119, the pertinent disclosures of which are incorporated herein by reference. Such hydrophobic but water-vapor permeable coatings are typically composed of hydrophobic polymers such as polyalkenes, polyacrylic acid derivatives, polyethers, polysulfones, polyethersulfones, polystyrenes, polyvinyl halides, polyvinyl esters and ethers, natural waxes and synthetic waxes. Especially preferred hydrophobic microporous coating materials include polystyrene, polysulfones, polyethersulfones, polyethylene, polypropylene, polyvinyl chloride, polyvinylidene fluoride and polytetrafluoroethylene. Such hydrophobic coatings can be made by known phase inversion methods using any of vapor-quench, liquid quench, thermal processes, leaching soluble material from the coating or by sintering coating particles. In thermal processes, a solution of polymer in a latent solvent is brought to liquid-liquid phase separation in a cooling step. When evaporation of the solvent is not prevented, the resulting membrane will typically be porous. Such coating processes may be conducted by the processes disclosed in U.S. Pat. Nos. 4,247,498; 4,490,431 and 4,744,906, the disclosures of which are also incorporated herein by reference.

Osmotic controlled-release devices may be prepared using procedures known in the pharmaceutical arts. See for example, Remington: The Science and Practice of Pharmacy, 20^{th} Edition, 2000.

### Multiparticulate Controlled Release Devices

The dosage forms of the present invention may also provide controlled release of the CETP inhibitor in solubility-improved form through the use of multiparticulate controlled release devices.

Multiparticulates generally refer to devices that comprise a multiplicity of particles or granules that may range in size from about 10 μm to about 2 mm, more typically about 100 μm to 1 mm in diameter. Such multiparticulates may be packaged, for example, in a capsule such as a gelatin capsule or a capsule formed from an aqueous-soluble polymer such as HPMCAS, HPMC or starch; dosed as a suspension or slurry in a liquid; or they may be formed into a tablet, caplet, or pill by compression or other processes known in the art.

Such multiparticulates may be made by any known process, such as wet- and dry-granulation processes, extrusion/spheronization, roller-compaction, melt-congealing, or by spray-coating seed cores. For example, in wet- and dry-granulation processes, the composition comprising the solubility-improved form of the CETP inhibitor and optional excipients may be granulated to form multiparticulates of the desired size. Other excipients, such as a binder (e.g., microcrystalline cellulose), may be blended with the composition to aid in processing and forming the multiparticulates. In the case of wet granulation, a binder such as HPC or PVP may be included in the granulation fluid to aid in forming a suitable multiparticulate. See, for example, Remington: The Science and Practice of Pharmacy, 20^{th} Edition, 2000.

In any case, the resulting particles may themselves constitute the multiparticulate device or they may be coated by various film-forming materials such as enteric polymers or water-swellable or water-soluble polymers, or they may be combined with other excipients or vehicles to aid in dosing to patients.

### Immediate-Release of an HMG CoA Reductase Inhibitor

The dosage forms of the present invention also provide immediate-release of an HMG-CoA reductase inhibitor. This means that the dosage form releases at least about 70 wt % of the HMG-CoA reductase inhibitor initially present in the dosage form within one hour or less following introduction to a use environment. Preferably, the dosage form releases at least about 80 wt % at one hour, and most preferably, at least about 90 wt % at one hour after administering the dosage form to a use environment.

Virtually any means for providing immediate release of the HMG-CoA reductase inhibitor known in the pharmaceutical arts can be used with the dosage form of the present invention. In one embodiment, the HMG-CoA reductase inhibitor is in the form of an immediate release coating that surrounds a composition containing the CETP inhibitor in solubility-improved form. The HMG-CoA reductase inhibitor may be combined with a water soluble or water dispersible polymer, such as HPC, HPMC, HEC, and the like. The coating can be formed using solvent-based coating processes, powder-coating processes, and hot-melt coating processes, all well known in the art. In solvent-based processes, the coating is made by first forming a solution or suspension comprising the solvent, the HMG-CoA reductase inhibitor, the coating polymer and optional coating additives. Preferably, the HMG-CoA reductase inhibitor is suspended in the coating solvent. The coating materials may be completely dissolved in the coating solvent, or only dispersed in the solvent as an emulsion or suspension or anywhere in between. Latex dispersions, including aqueous latex dispersions, are a specific example of an emulsion or suspension that may be useful as a coating solution. The solvent used for the solution should be inert in the sense that it does not react with or degrade the HMG-CoA reductase inhibitor, and be pharmaceutically acceptable. In one aspect, the solvent is a liquid at room temperature. Preferably, the solvent is a volatile solvent. By “volatile solvent” is meant that the material has a boiling point of less than about 150° C. at ambient pressure, although small amounts of solvents with higher boiling points can be used and acceptable results still obtained.

Examples of solvents suitable for use in applying a coating to a CETP inhibitor-containing core include alcohols, such as methanol, ethanol, isomers of propanol and isomers of butanol; ketones, such as acetone, methylethyl ketone and methyl isobutyl ketone; hydrocarbons, such as pentane, hexane, heptane, cyclohexane, methylcyclohexane, octane and mineral oil; ethers, such as methyl tert-butyl ether, ethyl ether and ethylene glycol monoethyl ether; chlorocarbons, such as chloroform, methylene dichloride and ethylene dichloride; tetrahydrofuran; dimethylsulfoxide; N-methylpyrrolidinone; acetonitrile; water; and mixtures thereof.

The coating formulation may also include additives to promote the desired immediate release characteristics or to ease the application or improve the durability or stability of the coating. Types of additives include plasticizers, pore formers, and glidants. Examples of coating additives suitable for use in the compositions of the present invention include plasticizers, such as mineral oils, petrolatum, lanolin alcohols, polyethylene glycol, polypropylene glycol, triethyl citrate, sorbitol, triethanol amine, diethyl phthalate, dibutyl phthalate, castor oil, triacetin and others known in the art; emulsifiers, such as polysorbate-80; pore formers, such as polyethylene glycol, polyvinyl pyrrolidone, polyethylene oxide, hydroxypropyl cellulose, hydroxyethyl cellulose and hydroxypropylmethyl cellulose; and glidants, such as colloidal silicon dioxide, talc and cornstarch. In one embodiment, the HMG-CoA reductase inhibitor is suspended in a commercially available coating formulation, such as Opadry® clear (available from Colorcon, Inc., WestPoint, Pa.). Coating is conducted in conventional fashion, typically by dipping, fluid-bed coating, spray-coating, or pan-coating.

The immediate release coating may also be applied using powder coating techniques well known in the art. In these techniques, the HMG-CoA reductase inhibitor is blended with optional coating excipients and additives, to form an HMG-CoA reductase inhibitor composition. This composition may then be applied using compression forces, such as in a tablet press.

The coating may also be applied using a hot-melt coating technique. In this method, a molten mixture comprising the HMG-CoA reductase inhibitor, and optional coating excipients and additives, is formed and then sprayed onto the composition containing the CETP inhibitor in solubility-improved form. Typically, the hot-melt coating is applied in a fluidized bed equipped with a top-spray arrangement.

In another embodiment, the HMG-CoA reductase inhibitor is first formed into an HMG-CoA reductase inhibitor composition comprising the HMG-CoA reductase inhibitor and optional excipients. This composition is then formed into an immediate-release layer, multiparticulates, powder or granules that are combined with the controlled-release CETP inhibitor device to form the dosage form of the current invention. In one aspect, the immediate-release HMG-CoA reductase inhibitor composition consists essentially of the HMG-CoA reductase inhibitor alone, such as crystalline drug. In another aspect, the immediate-release HMG-CoA reductase inhibitor composition comprises optional excipients, such as a stabilizing agents, diluents, disintegrants, and surfactants. The basic excipient, calcium carbonate, has been found to chemically stabilize HMG-CoA reductase inhibitors, such as atorvastatin calcium and pharmaceutically acceptable derivatives thereof. Microcrystalline cellulose and hydrous lactose are applied as suitable diluents. Croscarmellose sodium is present as a disintegrant. The non-ionic detergent Tween 80 is used as a surfactant. The composition may also contain hydroxypropyl cellulose as binder selected from among several applicable substances such as, i.e., polyethylene glycol, polyvinylpyrrolidone, polyvinyl alcohol, hydroxymethylcellulose or hydroxypropylmethylcellulose. As anti-oxidants, reagents such as butylated hydroxyanisole, sodium ascorbate, ascorbic acid or others may optionally be incorporated in the composition. Magnesium stearate can be selected from a group including other substances such as stearic acid, palmitic acid, talc or similar lubricating compounds.

Such immediate release HMG-CoA reductase inhibitor compositions may be formed by any conventional method for combining the HMG-CoA reductase inhibitor and excipients. Exemplary methods include wet and dry granulation. If wet granulation is used, a stabilizing agent such as calcium carbonate is preferably included to keep chemical degradation of the HMG-CoA reductase inhibitor at an acceptable level.

One exemplary method for forming the HMG-CoA reductase inhibitor composition comprises (a) milling the drug, (b) dissolving at least one binder additive in aqueous surfactant solution; (c) blending the milled drug with at least one drug-stabilizing additive and at least one diluent additive with the drug-stabilizing additive and one half of a disintegrant additive in a rotary mixing vessel equipped with a chopping device; (d) granulating the blended drug ingredient mixture of step (c) with the surfactant/binder solution of step (b) in gradual increments in the chopper equipped mixing vessel; (e) drying the granulated drug mixture overnight at about 50° C.; (f) sieving the dried granulated drug mixture; (g) tumble blending the sieved drug mixture with the remaining amount of the disintegrant additive; (h) mixing separately an aliquot of the drug mixture of step (g) with magnesium stearate, sieving same, and returning same to the drug mixture of step (g) and tumble blending the entire drug mixture.

In addition to the HMG-CoA reductase inhibitor, the immediate release layer may include other excipients to aid in formulating the composition into tablets, capsules, suspensions, powders for suspension, and the like. See, for example, Remington: The Science and Practice of Pharmacy (20th ed. 2000). Examples of other excipients include disintegrants, porosigens, matrix materials, fillers, diluents, lubricants, glidants, and the like, such as those previously described.

In one embodiment, the HMG-CoA reductase inhibitor composition also includes a base. The inclusion of a base can improve the chemical stability of the HMG-CoA reductase inhibitor. The term “base” is used broadly to include not only strong bases such as sodium hydroxide, but also weak bases and buffers that are capable of achieving the desired increase in chemical stability. Examples of bases include hydroxides, such as sodium hydroxide, calcium hydroxide, ammonium hydroxide, and choline hydroxide; bicarbonates, such as sodium bicarbonate, potassium bicarbonate, and ammonium bicarbonate; carbonates, such as ammonium carbonate, calcium carbonate, and sodium carbonate; amines, such as tris(hydroxymethyl)amino methane, ethanolamine, diethanolamine, N-methyl glucamine, glucosamine, ethylenediamine, N,N′-dibenzylethylenediamine, N-benzyl-2-phenethylamine, cyclohexylamine, cyclopentylamine, diethylamine, isopropylamine, diisopropylamine, dodecylamine, and triethylamine; proteins, such as gelatin; amino acids such as lysine, arginine, guanine, glycine, and adenine; polymeric amines, such as polyamino methacrylates, such as Eudragit E; conjugate bases of various acids, such as sodium acetate, sodium benzoate, ammonium acetate, disodium phosphate, trisodium phosphate, calcium hydrogen phosphate, sodium phenolate, sodium sulfate, ammonium chloride, and ammonium sulfate; salts of EDTA, such as tetra sodium EDTA; and salts of various acidic polymers such as sodium starch glycolate, sodium carboxymethyl cellulose and sodium polyacrylic acid.

### Immediate-Release Portion of the CETP Inhibitor

The dosage forms of the present invention also provide for a portion of the CETP inhibitor in solubility-improved form to be in an immediate-release form. In one embodiment, the immediate release portion of the CETP inhibitor in solubility-improved form can be included with the HMG-CoA reductase inhibitor composition as described above in immediate release coatings, layers, granules, multiparticulates or the like that are combined with the controlled release portion of the CETP inhibitor device to form the dosage form of the current invention. In another embodiment the immediate release portion of the CETP inhibitor in solubility-improved form is separated from the HMG-CoA reductase inhibitor composition. By immediate release means that the immediate release CETP inhibitor portion of the dosage form releases at least about 70 wt % of the immediate-release portion of the CETP inhibitor initially present in the dosage form within one hour or less following introduction to a use environment. Preferably, the dosage form releases at least about 80 wt % at one hour, and most preferably, at least about 90 wt % at one hour after administering the dosage form to a use environment.

Virtually any means for providing immediate release of the CETP inhibitor in solubility-improved form known in the pharmaceutical arts can be used with the dosage form of the present invention. In one embodiment, the CETP inhibitor in solubility-improved form is in the immediate release coating that surrounds a composition containing the CR portion of the CETP inhibitor in solubility-improved form. The CETP inhibitor may be combined with a water soluble or water dispersible polymer, such as HPC, HPMC, HEC, and the like. The coating can be formed using solvent-based coating processes, powder-coating processes, and hot-melt coating processes, all well known in the art. In solvent-based processes, the coating is made by first forming a solution or suspension comprising the solvent, the CETP inhibitor, the coating polymer and optional coating additives. Preferably, the CETP inhibitor in solubility-improved form is suspended in the coating solvent. The coating materials may be completely dissolved in the coating solvent, or only dispersed in the solvent as an emulsion or suspension or anywhere in between. Latex dispersions, including aqueous latex dispersions, are a specific example of an emulsion or suspension that may be useful as a coating solution. The solvent used for the solution should be inert in the sense that it does not react with or degrade the CETP inhibitor in solubility-improved form, and be pharmaceutically acceptable. Preferably, the solvent does not negatively impact the solubility-enhancing features of the solubility-enhancing form. In one aspect, the solvent is a liquid at room temperature. In one aspect, the solvent is a liquid at room temperature. Preferably, the solvent is a volatile solvent. By “volatile solvent” is meant that the material has a boiling point of less than about 150° C. at ambient pressure, although small amounts of solvents with higher boiling points can be used and acceptable results still obtained.

Examples of solvents suitable for use in applying an immediate release coating to a CR CETP inhibitor-containing core include alcohols, such as methanol, ethanol, isomers of propanol and isomers of butanol; ketones, such as acetone, methylethyl ketone and methyl isobutyl ketone; hydrocarbons, such as pentane, hexane, heptane, cyclohexane, methylcyclohexane, octane and mineral oil; ethers, such as methyl tert-butyl ether, ethyl ether and ethylene glycol monoethyl ether; chlorocarbons, such as chloroform, methylene dichloride and ethylene dichloride; tetrahydrofuran; dimethylsulfoxide; N-methyl pyrrolidinone; acetonitrile; water; and mixtures thereof.

The coating formulation may also include additives to promote the desired immediate release characteristics or to ease the application or improve the durability or stability of the coating. Types of additives include plasticizers, pore formers, and glidants. Examples of coating additives suitable for use in the compositions of the present invention include plasticizers, such as mineral oils, petrolatum, lanolin alcohols, polyethylene glycol, polypropylene glycol, triethyl citrate, sorbitol, triethanol amine, diethyl phthalate, dibutyl phthalate, castor oil, triacetin and others known in the art; emulsifiers, such as polysorbate-80; pore formers, such as polyethylene glycol, polyvinyl pyrrolidone, polyethylene oxide, hydroxyethyl cellulose and hydroxypropylmethyl cellulose; and glidants, such as colloidal silicon dioxide, talc and cornstarch. In one embodiment, the CETP inhibitor in solubility-improved form is suspended in a commercially available coating formulation, such as Opadry® clear (available from Colorcon, Inc., West Point, Pa.). Coating is conducted in conventional fashion, typically by dipping, fluid-bed coating, spray-coating, or pan-coating.

The immediate release coating may also be applied using powder coating techniques well known in the art. In these techniques, the CETP inhibitor in solubility-improved form is blended with optional coating excipients and additives, to form a CETP inhibitor composition. This composition may then be applied using compression forces, such as in a tablet press.

The coating may also be applied using a hot-melt coating technique. In this method, a molten mixture comprising the CETP inhibitor in solubility-improved form, and optional coating excipients and additives, is formed and then sprayed onto the composition containing the CR CETP inhibitor in solubility-improved form. Typically, the hot-melt coating is applied in a fluidized bed equipped with a top-spray arrangement.

In another embodiment, the CETP inhibitor is first formed into a CETP inhibitor composition comprising the CETP inhibitor in solubility-improved form and optional excipients. This composition is then formed into an immediate-release layer, multiparticulates, or granules that are combined with the controlled-release CETP inhibitor device to form the dosage form of the current invention. In one aspect, the immediate-release CETP inhibitor composition consists essentially of the CETP inhibitor in solubility-improved form alone. In another aspect, the immediate-release CETP inhibitor composition comprises optional excipients, such as a stabilizing agents, diluents, disintegrants, and surfactants. Microcrystalline cellulose and hydrous lactose are applied as suitable diluents. Croscarmellose sodium is present as a disintegrant. The non-ionic detergent Tween 80 is used as a surfactant. The composition may also contain hydroxypropyl cellulose as a binder selected from among several applicable substances such as, i.e., polyethylene glycol, polyvinylpyrrolidone, polyvinyl alcohol, hydroxymethylcellulose or hydroxypropylmethylcellulose. As anti-oxidants, reagents such as butylated hydroxyanisole, sodium ascorbate, ascorbic acid or others may optionally be incorporated in the composition. Magnesium stearate can be selected from a group including other substances such as stearic acid, palmitic acid, talc or similar lubricating compounds.

Such immediate release CETP inhibitor compositions may be formed by any conventional method for combining the CETP inhibitor in solubility-improved form and excipients. Exemplary methods include wet and dry granulation.

One exemplary method for forming the CETP inhibitor composition comprises (a) milling the CETP inhibitor in solubility-improved form, (b) dissolving at least one binder additive in aqueous surfactant solution; (c) blending the milled drug with at least one diluent additive and one half of a disintegrant additive in a rotary mixing vessel equipped with a chopping device; (d) granulating the blended drug ingredient mixture of step (c) with the surfactant/binder solution of step (b) in gradual increments in the chopper equipped mixing vessel; (e) drying the granulated drug mixture overnight at about 50° C.; (f) sieving the dried granulated drug mixture; (g) tumble blending the sieved drug mixture with the remaining amount of the disintegrant additive; (h) mixing separately an aliquot of the drug mixture of step (g) with magnesium stearate, sieving same, and returning same to the drug mixture of step (g) and tumble blending the entire drug mixture.

In addition to the CETP inhibitor in solubility-improved form, the immediate release layer may include other excipients to aid in formulating the composition into tablets, capsules, suspensions, powders for suspension, and the like. See, for example, Remington: The Science and Practice of Pharmacy (20th ed. 2000). Examples of other excipients include disintegrants, porosigens, matrix materials, fillers, diluents, lubricants, glidants, and the like, such as those previously described.

### Exemplary Embodiments

The dosage forms of the present invention comprise a CETP inhibitor in a solubility-improved form and an HMG-CoA reductase inhibitor. The amount of CETP inhibitor and HMG-CoA reductase inhibitor present in the dosage form will vary depending on the desired dose for each compound, which in turn, depends on the potency of the compound and the condition being treated. For example, the desired dose for the CETP inhibitor torcetrapib, also known as [2R,4S]-4-[(3,5-bis-trifluoromethyl-benzyl)-methoxycarbonyl-amino]-2-ethyl-6-trifluoromethyl-3,4-dihydro-2H-quinoline-1-carboxylic acid ethyl ester, ranges from 1 mg/day to 1000 mg/day, preferably 5 mg/day to 500 mg/day. For the HMG-CoA reductase inhibitor atorvastatin calcium, the dose ranges from 1 to 160 mg/day. For the HMG-CoA reductase inhibitors lovastatin, pravastatin sodium, simvastatin, rosuvastatin calcium, and fluvastatin sodium, the dose ranges from 2 to 160 mg/day. One skilled in the art will understand that the above dose ranges are exemplary for the drugs listed. It is intended that other CETP inhibitors and other HMG-CoA reductase inhibitors, including pharmaceutically acceptable forms of the above, be within the scope of the invention, and the dose of such compounds should be adjusted based on the potency and bioavailability of the drug.

In a specific preferred embodiment, the CETP inhibitor is torcetrapib and the HMG-CoA reductase inhibitor is atorvastatin calcium, also known as atorvastatin hemicalcium, or pharmaceutically acceptable forms thereof. For these compounds, it is preferred that the weight ratio of CETP inhibitor to HMG-CoA reductase inhibitor in the dosage form range from about 0.1 to about 36, preferably about 0.3 to about 20, more preferably about 0.5 to about 18.

The dosage forms of the present invention provide immediate release of the HMG-CoA reductase inhibitor and controlled and immediate release of the CETP inhibitor in solubility improved form. In one aspect, the dosage form is in the form of a unitary dosage form. By “unitary dosage form” is meant a single dosage form containing both the CETP inhibitor in solubility-improved form and the HMG-CoA reductase inhibitor so that, following administration of the unitary dosage form to a use environment, both the CETP inhibitor and HMG-CoA reductase inhibitor are delivered to the use environment, the HMG-CoA reductase inhibitor being delivered as immediate release and the CETP inhibitor being delivered as controlled release and immediate release. The term “unitary dosage form” includes a single tablet, caplet, pill, capsule, sachet, powder, solution, and a kit comprising one or more tablets, caplets, pills, capsules, sachets, powders, or solutions intended to be taken together.

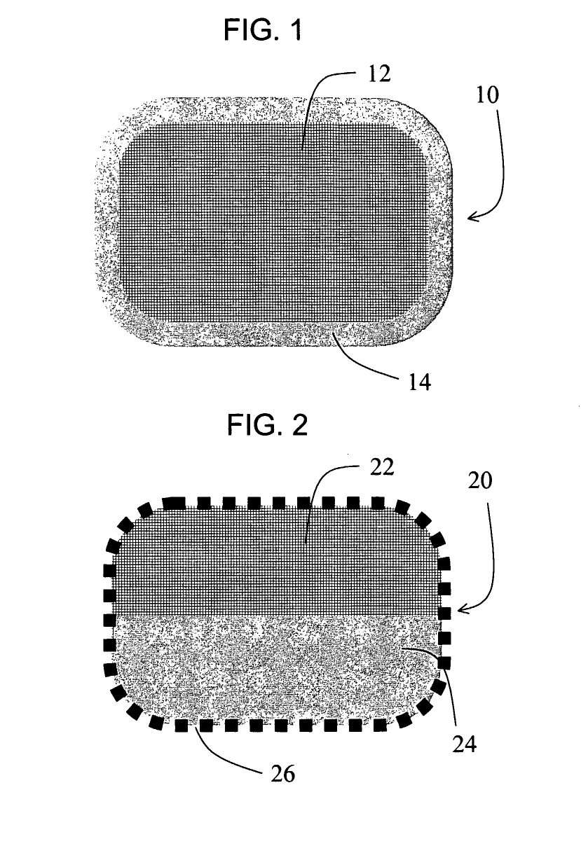

In one embodiment, the unitary dosage form comprises a CETP inhibitor composition and an HMG-CoA reductase inhibitor composition, wherein the CR portion of the CETP inhibitor composition is in the form of a matrix controlled release device and the HMG-CoA reductase inhibitor composition is in the form of an immediate release coating. The immediate release coating may include the immediate release portion of the CETP inhibitor in the same coating layer as the HMG-CoA reductase inhibitor or alternatively as a separate layer or a combination of separate and combined layers. The CR CETP inhibitor composition comprises the CETP inhibitor in solubility-improved form, a matrix polymer, and optional excipients as previously discussed for matrix controlled-release devices. The HMG-CoA reductase inhibitor composition comprises the HMG-CoA reductase inhibitor and optional excipients. Referring to FIG. 1, in one aspect, the unitary dosage form 10 is in the form of a matrix tablet 12 comprising the CETP inhibitor in solubility-improved form that is coated with an immediate release coating 14 comprising the HMG-CoA reductase inhibitor and optional excipients, as discussed above. Preferably, the solvent does not negatively impact the solubility-enhancing features of the solubility-enhancing form. The immediate release coating 14 may optionally be coated with a conventional coating (not shown in FIG. 1).

Alternatively, the unitary dosage form comprises a CETP inhibitor composition and an HMG-CoA reductase inhibitor composition, shown schematically as dosage form 20 in FIG. 2. The CETP inhibitor composition 22 is in the form of a matrix controlled release device and the HMG-CoA reductase inhibitor composition is in the form of an immediate release layer 24 associated with the matrix device. By associated with is meant that the layer comprising the HMG-CoA reductase inhibitor 24 is adjacent to or substantially in contact with the matrix controlled release device 22. The immediate release layer 24 may also be separated from the matrix controlled-release device by an intermediate layer (not shown in FIG. 2) comprising a binder or diluent, as known in the art. Preferably, the solvent does not negatively impact the solubility-enhancing features of the solubility-enhancing form. The unitary dosage form 20 may optionally be coated with a conventional coating 26. The immediate release coating may include the immediate release portion of the CETP inhibitor.

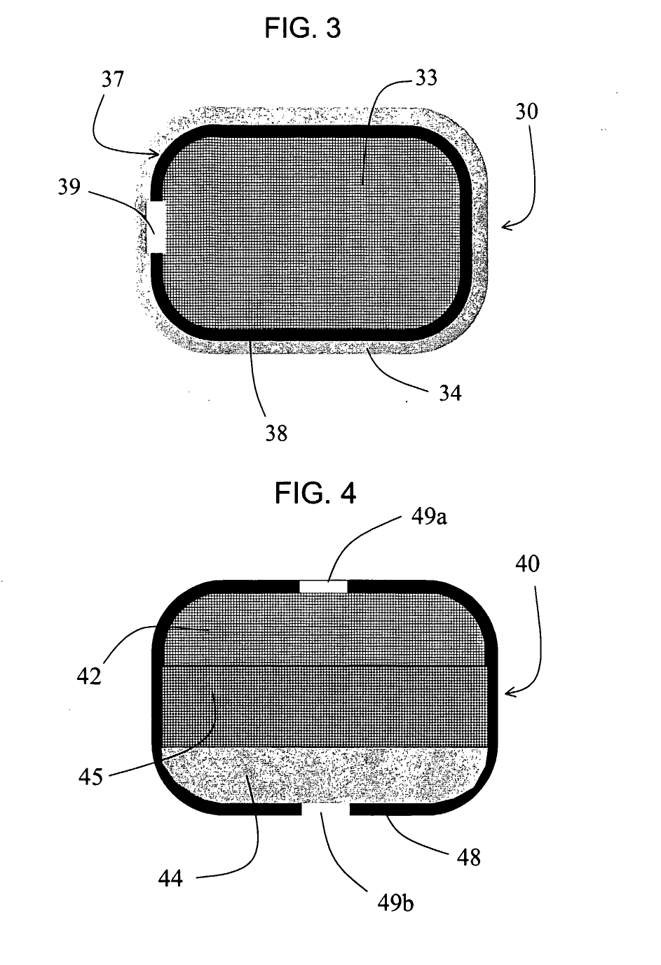

In another embodiment, the unitary dosage form comprises a CETP inhibitor composition and an HMG-CoA reductase inhibitor composition, shown schematically as dosage form 30 in FIG. 3. The CR CETP inhibitor composition is in the form of an osmotic controlled release device 37 and the HMG-CoA reductase inhibitor composition is in the form of an immediate release coating 34. The osmotic controlled release device 37 comprises a core 33, a coating 38, and a delivery port 39. The core may be a single composition, or may consist of several layers, including layers comprising the CETP inhibitor in solubility-improved form and highly swelling layers for extruding the CETP inhibitor into the use environment. The immediate release coating 34 may optionally be coated with a conventional coating (not shown in FIG. 3). The immediate release coating may include the immediate release portion of the CETP inhibitor in the same coating layer as the HMG-CoA reductase inhibitor or alternatively as a separate coating or a combination of separate and combined coatings.

In another embodiment, the unitary dosage form is in the form of a tri-layer tablet, shown schematically as dosage form 40 in FIG. 4. The tri-layer tablet comprises (1) a CETP inhibitor composition 42, (2) an HMG-CoA reductase inhibitor composition 44, (3) a sweller-layer composition 45 sandwiched between layers (1) and (2), (4) a water permeable coating 48 surround layers (1), (2), and (3), and (5) at least two delivery ports providing fluid communication between layer (1) and the use environment 49a and between layer (2) and the use environment 49b. The dosage form is designed such that the HMG-CoA reductase inhibitor composition 44 is released immediately following administration to the use environment, while the CETP inhibitor composition 42 is released slowly over time. The composition 44 contains a portion of an immediate release form of the CETP inhibitor in a solubility improved form.

In another embodiment, the unitary dosage form is in the form of a tri-layer tablet (not shown) comprising (1) an immediate release of the HMG-CoA reductase inhibitor composition, and (2) a controlled-release of the CETP inhibitor composition. A low-permeability coating is placed on the controlled-release CETP inhibitor composition. Such dosage forms are disclosed in U.S. Pat. Nos. 4,839,177, 5,422,123, 5,464,633, 5,650,169, 5,738,874 and 6,183,778, the disclosures of which are incorporated herein by reference.

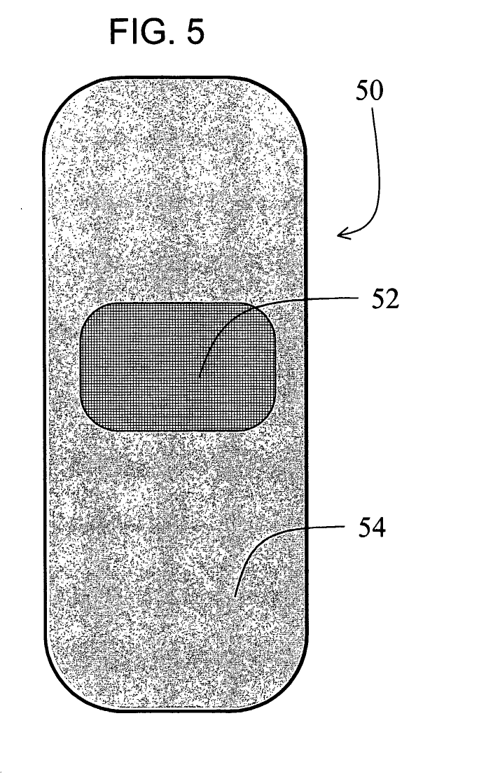

In another embodiment, the unitary dosage form is in the form of a capsule, the capsule, shown schematically as dosage form 50 in FIG. 5. The capsule comprises (1) at least one controlled-release device 52, such as a matrix controlled release device or an osmotic controlled release device, comprising the CETP inhibitor in solubility-improved form, and immediate release portion of the CETP inhibitor in a solubility-improved form, and (2) an immediate release HMG-CoA reductase inhibitor composition 54. In this embodiment, the controlled-release device 52 comprising the CETP inhibitor and the HMG-CoA reductase inhibitor composition 54 are first made using procedures known in the art, and then may be combined, such as by placing into a suitable capsule, such as a hard gelatin capsule or a soft gelatin capsule, well known in the art (see, for example, Remington: The Science and Practice of Pharmacy, (20th ed. 2000)). In one embodiment, the CETP inhibitor is in the form of a matrix controlled-release device previously discussed. In another embodiment, the CETP inhibitor is in the form of an osmotic controlled-release device, previously discussed. The immediate release HMG-CoA reductase inhibitor composition 54 may be simply particles of the active drug alone, or it may be combined with optional excipients and an immediate release portion of CETP inhibitor in solubility-improved form such that it is in the form of a powder, granules, or multiparticulates, previously described.

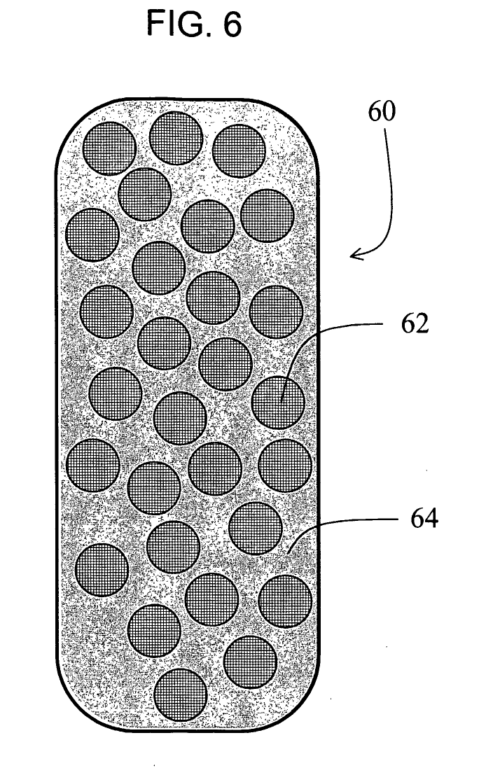

In another embodiment, the unitary dosage form is in the form of a capsule, shown schematically as dosage form 60 in FIG. 6. The capsule comprises (1) a plurality of controlled-release devices, such as controlled-release multiparticulates or granules 62 comprising the CETP inhibitor in solubility-improved form, and (2) an immediate release HMG-CoA reductase inhibitor composition 64. The controlled-release CETP inhibitor multiparticulates or granules 62 and HMG-CoA reductase inhibitor composition 64 are first made using the procedures previously outlined, and then may be combined, such as by placing them into a suitable capsule, such as a hard gelatin capsule or a soft gelatin capsule, well known in the art (see, for example, Remington: The Science and Practice of Pharmacy, (20th ed. 2000)). Composition 64 may also include an immediate release portion of CETP inhibitor in solubility-improved form.

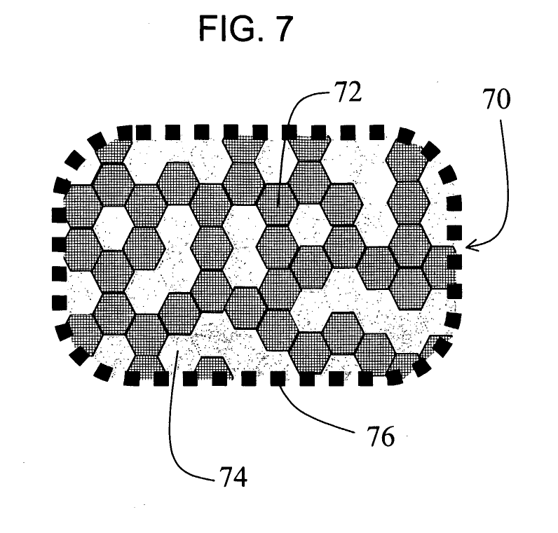

In yet another embodiment, the unitary dosage form is in the form of a compressed tablet, caplet, or pill, shown schematically as dosage form 70 in FIG. 7. The dosage form comprises (1) a plurality of controlled-release multiparticulates or granules 72 comprising the CETP inhibitor in solubility-improved form, and (2) a plurality of particles that immediately release the HMG-CoA reductase inhibitor, such as particles of active drug alone, or multiparticulates or granules 74 comprising the HMG-CoA reductase inhibitor. Multiparticulates or granules 74 may also include an immediate release portion of CETP inhibitor in solubility-improved form.

The unitary dosage form may optionally be coated with a conventional coating 76 or with a immediate release coating containing HMG-CoA reductase inhibitor or an immediate release portion of CETP inhibitor in solubility-improved form or both drugs

Yet another embodiment of the unitary dosage form is a powder or granulation, often referred to in the art as a sachet or oral powder for constitution (OPC). Controlled release granules or multiparticulates of the CETP inhibitor in solubility-improved form and particles that immediately release the HMG-CoA reductase inhibitor, such as particles of active drug alone, or granules or multiparticulates comprising the HMG-CoA reductase inhibitor, are mixed with optional excipients or an immediate release portion of CETP inhibitor in solubility-improved form and placed into a suitable container, such as a pouch, bottle, box, bag, or other container known in the art. The powder dosage form can then be taken dry or mixed with a liquid to form a paste, suspension or slurry prior to dosing.

Yet another embodiment of the unitary dosage form is a kit comprising at least two separate compositions: (1) one containing a controlled release device comprising the CETP inhibitor in solubility-improved form, and (2) one containing the HMG-CoA reductase inhibitor in immediate release form and the CETP inhibitor in immediate release form, or alternatively, three separate compositions: (1) one containing a controlled release device comprising the CETP inhibitor in solubility-improved form, (2) one containing the HMG-CoA reductase inhibitor in immediate release form and (3) one containing the CETP inhibitor in immediate release form. The kit may include means for containing the separate compositions such as a divided container, such as a bottle, pouch, box, bag, or other container known in the art, or a divided foil packet; however, the separate compositions may also be contained within a single, undivided container. Typically the kit includes directions for the administration of the separate components.

In another embodiment, the CETP inhibitor in solubility-improved form and the HMG-CoA reductase inhibitor are present in separate dosage forms that are co-administered to the environment of use. The CETP inhibitor in solubility-improved form is in a controlled release dosage form, while the HMG-CoA reductase inhibitor is in an immediate release dosage form. In another aspect of the embodiment the immediate release dosage form could also include an immediate release portion of the CETP inhibitor in solubility-improved form. By “co-administered” is meant that the two dosage forms are administered separately from each other. In one embodiment, the two dosage forms are co-administered within the same general time frame as each other, such as within 60 minutes, preferably within 30 minutes, more preferably within 15 minutes of each other. In another embodiment, the two dosage forms are taken at separate times. For example, the dosage form comprising the controlled release and immediate release CETP inhibitor in solubility-improved form may be taken at meal time, for example, breakfast, lunch, or dinner, while the immediate-release dosage form comprising the HMG-CoA reductase inhibitor is taken in the evening. Either of these scenarios or variations on these scenarios are considered within the scope of the invention.

The invention also covers a method of treating a subject in need of CETP inhibitor and/or HMG-CoA reductase inhibitor therapy comprising administering to a subject in need of such therapy a dosage form of the present invention. The dosage form provides at least one of: (i) at least about 50% inhibition of plasma cholesteryl ester transfer protein for at least about 12 hours; (ii) a maximum drug concentration in the blood that is less than or equal to about 80% of the maximum drug concentration in the blood provided by a dosage form that provides immediate release of the same amount of the solubility-improved form of said CETP inhibitor; (iii) a mean HDL cholesterol level after dosing for 8 weeks that is at least about 1.2-fold that obtained prior to dosing; and (iv) a mean LDL cholesterol level after dosing for 8 weeks that is less than or equal to about 90% that obtained prior to dosing.

The dosage forms of the present invention may optionally be coated with a conventional coating well known in the art. The coatings may be used to mask taste, improve appearance, facilitate swallowing of the dosage form, or to delay, sustain or otherwise control the release of the drug from the dosage form. Such coatings may be fabricated by any conventional means including fluidized bed coating, spray-coating, dip-coating, pan-coating and powder-coating using aqueous or organic solvents. Examples of suitable coating materials include sucrose, maltitol, cellulose acetate, ethyl cellulose, methylcellulose, sodium carboxymethyl cellulose, hydroxyethyl cellulose, hydroxypropyl cellulose, hydroxypropyl methylcellulose, polymethacrylates, polyacrylates, polyvinyl alcohol, polyvinyl pyrrolidone, cetyl alcohol, gelatin, maltodextrin, paraffin wax, microcrystalline wax, and carnauba wax. Mixtures of polymers may also be used. Preferred coatings include the commercial aqueous coating formulations Surelease® and Opadry® available from Colorcon Inc. (West Point, Pa.).

### Cholesteryl Ester Transfer Protein Inhibitors

The CETP inhibitor may be any compound capable of inhibiting the cholesteryl ester transfer protein. The CETP inhibitor is typically “sparingly water-soluble,” which means that the CETP inhibitor has a minimum aqueous solubility of less than about 1 to 2 mg/mL at any physiologically relevant pH (e.g., pH 1-8) and at about 22° C. Many CETP inhibitors are “substantially water-insoluble,” which means that the CETP inhibitor has a minimum aqueous solubility of less than about 0.01 mg/mL (or 10 μg/mL) at any physiologically relevant pH (e.g., pH 1-8) and at about 22° C. (Unless otherwise specified, reference to aqueous solubility herein and in the claims is determined at about 22° C.) Compositions of the present invention find greater utility as the solubility of the CETP inhibitors decreases, and thus are preferred for CETP inhibitors with solubilities less than about 10 μg/mL, and even more preferred for CETP inhibitors with solubilities less than about 1 μg/mL. Many CETP inhibitors have even lower solubilities (some even less than 0.1 μg/mL), and require dramatic concentration enhancement to be sufficiently bioavailable upon oral dosing for effective plasma concentrations to be reached at practical doses.

In general, the CETP inhibitor has a dose-to-aqueous solubility ratio greater than about 100 mL, where the solubility (mg/mL) is the minimum value observed in any physiologically relevant aqueous solution (e.g., those with pH values from 1 to 8) including USP simulated gastric and intestinal buffers, and dose is in mg. Compositions of the present invention, as mentioned above, find greater utility as the solubility of the CETP inhibitor decreases and the dose increases. Thus, the compositions are preferred as the dose-to-solubility ratio increases, and thus are preferred for dose-to-solubility ratios greater than 1000 mL, and more preferred for dose-to-solubility ratios greater than about 5000 mL. The dose-to-solubility ratio may be determined by dividing the dose (in mg) by the aqueous solubility (in mg/mL).

Oral delivery of many CETP inhibitors is particularly difficult because their aqueous solubility is usually extremely low, typically being less than 2 μg/mL, often being less than 0.1 μg/mL. Such low solubilities are a direct consequence of the particular structural characteristics of species that bind to CETP and thus act as CETP inhibitors. This low solubility is primarily due to the hydrophobic nature of CETP inhibitors. Clog P, defined as the base 10 logarithm of the ratio of the drug solubility in octanol to the drug solubility in water, is a widely accepted measure of hydrophobicity. In general, Clog P values for CETP inhibitors are greater than 4 and are often greater than 5. Thus, the hydrophobic and insoluble nature of CETP inhibitors as a class pose a particular challenge for oral delivery. Achieving therapeutic drug levels in the blood by oral dosing of practical quantities of drug generally requires a large enhancement in drug concentrations in the gastrointestinal fluid and a resulting large enhancement in bioavailability. Such enhancements in drug concentration in gastrointestinal fluid typically need to be at least about 10-fold and often at least about 50-fold or even at least about 200-fold to achieve desired blood levels.

The inventors have recognized a subclass of CETP inhibitors that are essentially aqueous insoluble, highly hydrophobic, and are characterized by a set of physical properties. The first property of this subclass of essentially insoluble, hydrophobic CETP inhibitors is extremely low aqueous solubility. By extremely low aqueous solubility is meant that the minimum aqueous solubility at physiologically relevant pH (pH of 1 to 8) is less than about 10 μg/mL and preferably less than about 1 μg/mL.

A second property is a very high dose-to-solubility ratio. Extremely low aqueous solubility often leads to poor or slow absorption of the drug from the fluid of the gastrointestinal tract, when the drug is dosed orally in a conventional manner. For extremely low solubility drugs, poor absorption generally becomes progressively more difficult as the dose (mass of drug given orally) increases. Thus, a second property of this subclass of essentially insoluble, hydrophobic CETP inhibitors is a very high dose (in mg) to solubility (in mg/mL) ratio (mL). By “very high dose-to-solubility ratio” is meant that the dose-to-solubility ratio has a value of at least about 1000 mL, and preferably at least about 5,000 mL, and more preferably at least about 10,000 mL.

A third property of this subclass of essentially insoluble, hydrophobic CETP inhibitors is that they are extremely hydrophobic. By extremely hydrophobic is meant that the Clog P value of the drug, has a value of at least about 4.0, preferably a value of at least about 5.0, and more preferably a value of at least about 5.5.

A fourth property of this subclass of essentially insoluble CETP inhibitors is that they have a low melting point. Generally, drugs of this subclass will have a melting point of about 150° C. or less, and preferably about 140° C. or less.

Primarily, as a consequence of some or all of these four properties, CETP inhibitors of this subclass typically have very low absolute bioavailabilities. Specifically, the absolute bioavailability of drugs in this subclass when dosed orally in their undispersed state is less than about 10% and more often less than about 5%.

In the following, by “pharmaceutically acceptable forms” thereof is meant any pharmaceutically acceptable derivative or variation, including stereoisomers, stereoisomer mixtures, enantiomers, solvates, hydrates, isomorphs, pseudomorphs, polymorphs, salt forms and prodrugs.

In a preferred embodiment, the CETP inhibitor is [2R,4S]-4-[(3,5-bis-trifluoromethyl-benzyl)-methoxycarbonyl-amino]-2-ethyl-6-trifluoromethyl-3,4-dihydro-2H-quinoline-1-carboxylic acid ethyl ester also known as torcetrapib. Torcetrapib is shown by the following Formula

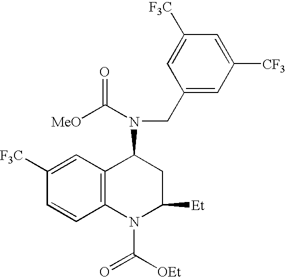

CETP inhibitors, in particular torcetrapib, and methods for preparing such compounds are disclosed in detail in U.S. Pat. Nos. 6,197,786 and 6,313,142, in PCT Application Nos. WO 01/40190A1, WO 02/088085A2, and WO 02/088069A2, the disclosures of which are herein incorporated by reference. Torcetrapib has an unusually low solubility in aqueous environments such as the lumenal fluid of the human GI tract. The aqueous solubility of torcetrapib is less than about 0.04 μg/mL. Torcetrapib must be presented to the GI tract in a solubility-improved form in order to achieve a sufficient drug concentration in the GI tract in order to achieve sufficient absorption into the blood to elicit the desired therapeutic effect.

CETP inhibitors are also described in U.S. Pat. No. 6,723,752, which includes a number of CETP inhibitors including (2R)-3-{[3-(4-Chloro-3-ethyl-phenoxy)-phenyl]-[[3-(1,1,2,2-tetrafluoro-ethoxy)-phenyl]-methyl]-amino}-1,1,1-trifluoro-2-propanol. Moreover, CETP inhibitors included herein are also described in U.S. patent application Ser. No. 10/807,838 filed Mar. 23, 2004, and U.S. Patent Application No. 60/612,863, filed Sep. 23, 2004, which includes (2R,4R,4aS)-4-[Amino-(3,5-bis-(trifluoromethyl-phenyl)-methyl]-2-ethyl-6-(trifluoromethyl)-3,4-dihydroquinoline-1-carboxylic acid isopropyl ester. Further CETP inhibitors include JTT-705, also known as S-[2-([[1-(2-ethyl butyl)cyclohexyl]carbonyl]amino)phenyl]2-methylpropanethioate, and those compounds disclosed in PCT Application No. WO04/020393, such as S-[2-([[1-(2-ethylbutyl)cyclohexyl]carbonyl]amino)phenyl]2-methylpropanethioate, trans-4-[[[2-[[[[3,5-bis(trifluoromethyl)phenyl]methyl](2-methyl-2H-tetrazol-5-yl)amino]methyl]-4-(trifluoromethyl)phenyl]ethylamino]methyl]-cyclohexaneacetic acid and trans-4-[[[2-[[[[3,5-bis(trifluoromethyl)phenyl]methyl](2-methyl-2H-tetrazol-5-yl)amino]methyl]-5-methyl-4-(trifluoromethyl)phenyl]ethylamino]methyl]-cyclohexaneacetic acid, the drugs disclosed in commonly owned U.S. patent application Ser. Nos. 09/918,127 and 10/066,091, the disclosures of both of which are incorporated herein by reference, and the drugs disclosed in the following patents and published applications, the disclosures of all of which are incorporated herein by reference: DE 19741400 A1; DE 19741399 A1; WO 9914215 A1; WO 9914174; DE 19709125 A1; DE 19704244 A1; DE 19704243 A1; EP 818448 A1; WO 9804528 A2; DE 19627431 A1; DE 19627430 A1; DE 19627419 A1; EP 796846 A1; DE 19832159; DE 818197; DE 19741051; WO 9941237 A1; WO 9914204 A1; WO 9835937 A1; JP 11049743; WO 0018721; WO 0018723; WO 0018724; WO 0017164; WO 0017165; WO 0017166; WO 04020393; EP 992496; and EP 987251.

### Solubility-Improved Forms

The solubility-improved form of the CETP inhibitor is any form that is capable of supersaturating, at least temporarily, in an aqueous use environment by a factor of about 1.25-fold or more, relative to the solubility of crystalline CETP inhibitor. That is, the solubility-improved form provides a maximum dissolved drug concentration (MDC) of the CETP inhibitor in a use environment that is at least about 1.25-fold the equilibrium drug concentration provided by the crystalline form of the CETP inhibitor alone. Preferably, the solubility-improved form increases the MDC of the CETP inhibitor in aqueous solution by at least about 2-fold relative to a control composition, more preferably by at least about 3-fold, and most preferably by at least about 5-fold. Surprisingly, the solubility-improved form may achieve extremely large enhancements in aqueous concentration. In some cases, the MDC of CETP inhibitor provided by the solubility-improved form is at least about 10-fold, at least about 50-fold, at least about 200-fold, at least about 500-fold, to more than 1000-fold the equilibrium concentration provided by the control.

Alternatively, the solubility-improved form provides an area under the drug concentration versus time curve (“AUC”) in the use environment that may be at least about 1.25-fold that provided by a control composition. The AUC is the integration of a plot of the drug concentration versus time. When the use environment is in vitro, the AUC can be determined by plotting the drug concentration in the test solution over time or for in vivo tests by plotting the drug concentration in the in vivo use environment (such as the GI tract of an animal) over time. The calculation of an AUC is a well-known procedure in the pharmaceutical arts and is described, for example, in Welling, “Pharmacokinetics Processes and Mathematics,” ACS Monograph 185 (1986). More specifically, in the environment of use, the CETP inhibitor in solubility-improved form provides an AUC for any 90-minute period of from about 0 to about 270 minutes following introduction to the use environment that is at least about 1.25-fold that of a control composition. The control composition is conventionally the lowest-energy crystalline form of the CETP inhibitor alone without any solubilizing additives. It is to be understood that the control composition is free from solubilizers or other components that would materially affect the solubility of the CETP inhibitor, and that the CETP inhibitor is in solid form in the control composition. The control composition is conventionally the lowest energy or most stable crystalline form of the CETP inhibitor alone, otherwise referred to hereinafter and in the claims as CETP inhibitor in “bulk crystalline form.” Preferably, the AUC provided by the solubility-improved form is at least about 2-fold, more preferably at least about 3-fold that of the control composition. For some CETP inhibitors, the solubility-improved form may provide an AUC value that is at least about 5-fold, at least about 25-fold, at least about 100-fold, and even more than 250-fold that of the control described above.

The solubility-improved form may comprise a solid amorphous dispersion of the CETP inhibitor in a concentration-enhancing polymer or low molecular weight water-soluble material. Solid amorphous dispersions of CETP inhibitors and concentration-enhancing polymers are disclosed more fully in commonly assigned U.S. patent application Ser. No. 09/918,127, filed Jul. 30, 2001, and U.S. patent application Ser. No. 10/066,091, filed Feb. 1, 2002, both of which are herein incorporated by reference. Alternatively, the solubility-improved form may comprise amorphous CETP inhibitor. The solubility-improved form may comprise nanoparticles, i.e. solid CETP inhibitor particles of diameter less than approximately 900 nm, optionally stabilized by small quantities of surfactants or polymers, as described in U.S. Pat. No. 5,145,684. The solubility-improved form may comprise adsorbates of the CETP inhibitor in a crosslinked polymer, as described in U.S. Pat. No. 5,225,192. The solubility-improved form may comprise a nanosuspension, the nanosuspension being a disperse system of solid-in-liquid or solid-in-semisolid, the dispersed phase comprising pure CETP inhibitor or a CETP inhibitor mixture, as described in U.S. Pat. No. 5,858,410. The solubility-improved form may comprise CETP inhibitor that is in a supercooled form, as described in U.S. Pat. No. 6,197,349. The solubility-improved form may comprise a CETP inhibitor/cyclodextrin form, including those described in U.S. Pat. Nos. 5,134,127, 6,046,177, 5,874,418, and 5,376,645. The solubility-improved form may comprise a softgel form, such as a CETP inhibitor mixed with a lipid or colloidal protein (e.g., gelatin), including those described in U.S. Pat. Nos. 5,851,275, 5,834,022 and 5,686,133. The solubility-improved form may comprise a self-emulsifying form, including those described in U.S. Pat. Nos. 6,054,136 and 5,993,858. The solubility-improved form may comprise a three-phase drug form, including those described in U.S. Pat. No. 6,042,847. The above solubility-improved forms may also be mixed with a concentration-enhancing polymer to provide improved solubility enhancements, as disclosed in commonly assigned copending U.S. patent application Ser. No. 10/176,462 filed Jun. 20, 2002, which is incorporated in its entirety by reference. The solubility-improved form may also comprise (1) a crystalline highly soluble form of the CETP inhibitor such as a salt; (2) a high-energy crystalline form of the CETP inhibitor; (3) a hydrate or solvate crystalline form of a CETP inhibitor; (4) an amorphous form of a CETP inhibitor (for a CETP inhibitor that may exist as either amorphous or crystalline); (5) a mixture of the CETP inhibitor (amorphous or crystalline) and a solubilizing agent; or (6) a solution of the CETP inhibitor dissolved in an aqueous or organic liquid. The above solubility-improved forms may also be mixed with a concentration-enhancing polymer to provide improved solubility enhancements, as disclosed in commonly assigned copending U.S. patent application Ser. No. 09/742,785 filed Dec. 20, 2000, which is incorporated in its entirety by reference. The solubility-improved form may also comprise (a) a solid dispersion comprising a CETP inhibitor and a matrix, wherein at least a major portion of the CETP inhibitor in the dispersion is amorphous; and (b) a concentration-enhancing polymer, as disclosed in commonly assigned copending U.S. Provisional Patent Application Ser. No. 60/300,261, filed Jun. 22, 2001, which is incorporated in its entirety by reference. The solubility-improved form may also comprise a solid adsorbate comprising a low-solubility CETP inhibitor adsorbed onto a substrate, the substrate having a surface area of at least about 20 m²/g, and wherein at least a major portion of the CETP inhibitor in the solid adsorbate is amorphous. The solid adsorbate may optionally comprise a concentration-enhancing polymer. The solid adsorbate may also be mixed with a concentration-enhancing polymer. Such solid adsorbates are disclosed in commonly assigned copending U.S. patent application Ser. No. 10/173,987, filed Jun. 17, 2002, which is incorporated in its entirety by reference. The solubility-improved form may also comprise a CETP inhibitor formulated in a lipid vehicle of the type disclosed in commonly assigned copending U.S. patent application Ser. No. 10/175,643 filed on Jun. 19, 2002, which is also incorporated in its entirety by reference.

The aqueous use environment can be either the in vivo environment, such as the GI tract of an animal, particularly a human, or the in vitro environment of a test solution, such as phosphate buffered saline (PBS) solution or Model Fasted Duodenal (MFD) solution.

The solubility-improved forms of CETP inhibitor used in the inventive dosage forms provide enhanced concentration of the dissolved CETP inhibitor in in vitro dissolution tests. It has been determined that enhanced drug concentration in in vitro dissolution tests in MFD solution or in PBS solution is a good indicator of in vivo performance and bioavailability. An appropriate PBS solution is an aqueous solution comprising 20 mM Na₂HPO₄, 47 mM KH₂PO₄, 87 mM NaCl, and 0.2 mM KCl, adjusted to pH 6.5 with NaOH. An appropriate MFD solution is the same PBS solution wherein there is also present 7.3 mM sodium taurocholic acid and 1.4 mM of 1-palmitoyl-2-oleyl-sn-glycero-3-phosphocholine. In particular, the CETP inhibitor in solubility-improved form can be dissolution-tested by adding it to MFD or PBS solution and agitating to promote dissolution.

An in vitro test to evaluate enhanced CETP inhibitor concentration in aqueous solution can be conducted by (1) adding with agitation a sufficient quantity of control composition, i.e., the CETP inhibitor in bulk crystalline form alone, to the in vitro test medium, such as an MFD or a PBS solution, to achieve equilibrium concentration of the CETP inhibitor; (2) in a separate test, adding with agitation a sufficient quantity of test composition (e.g., the CETP inhibitor in solubility-improved form) in the same test medium, such that if all the CETP inhibitor dissolved, the theoretical concentration of CETP inhibitor would exceed the equilibrium concentration of the CETP inhibitor by a factor of at least about 2, and preferably by a factor of at least about 10; and (3) comparing the measured MDC and/or aqueous AUC of the test composition in the test medium with the equilibrium concentration, and/or with the aqueous AUC of the control composition. In conducting such a dissolution test, the amount of test composition or control composition used is an amount such that if all of the CETP inhibitor dissolved the CETP inhibitor concentration would be at least about 2-fold, and preferably at least about 100-fold that of the equilibrium concentration. Indeed, for some extremely insoluble CETP inhibitors, in order to identify the MDC achieved it may be necessary to use an amount of test composition such that if all of the CETP inhibitor dissolved, the CETP inhibitor concentration would be 1000-fold or even more, that of the equilibrium concentration of the CETP inhibitor.

The concentration of dissolved CETP inhibitor is typically measured as a function of time by sampling the test medium and plotting CETP inhibitor concentration in the test medium vs. time so that the MDC can be ascertained. The MDC is taken to be the maximum value of dissolved CETP inhibitor measured over the duration of the test. The aqueous AUC is calculated by integrating the concentration versus time curve over any 90-minute time period between the time of introduction of the composition into the aqueous use environment (when time equals zero) and 270 minutes following introduction to the use environment (when time equals 270 minutes). Typically, when the composition reaches its MDC rapidly, in say less than about 30 minutes, the time interval used to calculate AUC is from time equals zero to time equals 90 minutes. However, if the AUC of a composition over any 90-minute time period described above meets the criterion of this invention, then the composition formed is considered to be within the scope of this invention.

To avoid large CETP inhibitor particulates that would give an erroneous determination, the test solution is either filtered or centrifuged. “Dissolved drug” is typically taken as that material that either passes a 0.45 μm syringe filter or, alternatively, the material that remains in the supernatant following centrifugation. Filtration can be conducted using a 13 mm, 0.45 μm polyvinylidine difluoride syringe filter sold by Scientific Resources under the trademark TITAN®. Centrifugation is typically carried out in a polypropylene microcentrifuge tube by centrifuging at 13,000 G for 60 seconds. Other similar filtration or centrifugation methods can be employed and useful results obtained. For example, using other types of microfilters may yield values somewhat higher or lower (±10-40%) than that obtained with the filter specified above but will still allow identification of preferred dispersions.

Alternatively, the CETP inhibitor in solubility-improved form, when dosed orally to a human or other animal, provides an AUC in CETP inhibitor concentration in the blood (serum or plasma) that is at least about 1.25-fold, preferably at least about 2-fold, preferably at least about 3-fold, preferably at least about 4-fold, preferably at least about 6-fold, preferably at least about 10-fold, and even more preferably at least about 20-fold that observed when a control composition consisting of an equivalent quantity of CETP inhibitor in bulk crystalline form is dosed. It is noted that such compositions can also be said to have a relative bioavailability of from about 1.25-fold to about 20-fold that of the control composition.

Relative bioavailability of CETP inhibitors in solubility-improved form can be tested in vivo in animals or humans using conventional methods for making such a determination. An in vivo test, such as a crossover study, may be used to determine whether a composition of CETP inhibitor in solubility-improved form provides an enhanced relative bioavailability compared with a control composition as described above. In an in vivo crossover study a test composition of a CETP inhibitor in solubility-improved form is dosed to half a group of test subjects and, after an appropriate washout period (e.g., one week) the same subjects are dosed with a control composition that consists of an equivalent quantity of crystalline CETP inhibitor as the test composition. The other half of the group is dosed with the control composition first, followed by the test composition. The relative bioavailability is measured as the concentration in the blood (serum or plasma) versus time area under the curve (AUC) determined for the test group divided by the AUC in the blood provided by the control composition. Preferably, this test/control ratio is determined for each subject, and then the ratios are averaged over all subjects in the study. In vivo determinations of AUC can be made by plotting the serum or plasma concentration of drug along the ordinate (y-axis) against time along the abscissa (x-axis). To facilitate dosing, a dosing vehicle may be used to administer the dose. The dosing vehicle is preferably water, but may also contain materials for suspending the test or control composition, provided these materials do not dissolve the composition or change the drug solubility in vivo.

### Solid Amorphous Dispersions of CETP Inhibitors

In one embodiment, the CETP inhibitor in a solubility-improved form comprises a solid amorphous dispersion of the CETP inhibitor and a concentration-enhancing polymer. By solid amorphous dispersion is meant a solid material in which at least a portion of the CETP inhibitor is in the amorphous form and dispersed in the polymer. Preferably, at least a major portion of the CETP inhibitor in the solid amorphous dispersion is amorphous. By “amorphous” is meant simply that the CETP inhibitor is in a non-crystalline state. As used herein, the term “a major portion” of the CETP inhibitor means that at least about 60 wt % of the drug in the solid amorphous dispersion is in the amorphous form, rather than the crystalline form. Preferably, the CETP inhibitor in the solid amorphous dispersion is substantially amorphous. As used herein, “substantially amorphous” means that the amount of the CETP inhibitor in crystalline form does not exceed about 25 wt %. More preferably, the CETP inhibitor in the solid amorphous dispersion is “almost completely amorphous,” meaning that the amount of CETP inhibitor in the crystalline form does not exceed about 10 wt %. Amounts of crystalline CETP inhibitor may be measured by Powder X-Ray Diffraction (PXRD), Scanning Electron Microscope (SEM) analysis, differential scanning calorimetry (DSC), or any other standard quantitative measurement.

The solid amorphous dispersions may contain from about 1 to about 80 wt % CETP inhibitor, depending on the dose of the CETP inhibitor and the effectiveness of the concentration-enhancing polymer. Enhancement of aqueous CETP inhibitor concentrations and relative bioavailability are typically best at low CETP inhibitor levels, typically less than about 25 to about 40 wt %. However, due to the practical limit of the dosage form size, higher CETP inhibitor levels may be preferred and in many cases perform well.

The amorphous CETP inhibitor can exist within the solid amorphous dispersion in relatively pure amorphous drug domains or regions, as a solid solution of drug homogeneously distributed throughout the polymer or any combination of these states or those states that lie intermediate between them. The solid amorphous dispersion is preferably substantially homogeneous so that the amorphous CETP inhibitor is dispersed as homogeneously as possible throughout the polymer. As used herein, “substantially homogeneous” means that the fraction of CETP inhibitor that is present in relatively pure amorphous drug domains or regions within the solid amorphous dispersion is relatively small, on the order of less than about 20 wt %, and preferably less than about 10 wt % of the total amount of drug. Solid amorphous dispersions that are substantially homogeneous generally are more physically stable and have improved concentration-enhancing properties and, in turn, improved bioavailability, relative to nonhomogeneous dispersions.

In cases where the CETP inhibitor and the polymer have glass transition temperatures sufficiently far apart (greater than about 20° C.), the fraction of drug that is present in relatively pure amorphous drug domains or regions within the solid amorphous dispersion can be determined by examining the glass transition temperature (T_{g}) of the solid amorphous dispersion. T_{g} as used herein is the characteristic temperature where a glassy material, upon gradual heating, undergoes a relatively rapid (e.g., in 10 to 100 seconds) physical change from a glassy state to a rubbery state. The T_{g} of an amorphous material such as a polymer, drug, or dispersion can be measured by several techniques, including by a dynamic mechanical analyzer (DMA), a dilatometer, a dielectric analyzer, and by DSC. The exact values measured by each technique can vary somewhat, but usually fall within 100 to 30° C. of each other. When the solid amorphous dispersion exhibits a single T_{g}, the amount of CETP inhibitor in pure amorphous drug domains or regions in the solid amorphous dispersion is generally less than about 10 wt %, confirming that the solid amorphous dispersion is substantially homogeneous. This is in contrast to a simple physical mixture of pure amorphous drug particles and pure amorphous polymer particles which generally display two distinct T_{g}s, one being that of the drug and one that of the polymer. For a solid amorphous dispersion that exhibits two distinct T_{g}s, one in the proximity of the drug T_{g} and one of the remaining drug/polymer dispersion, at least a portion of the drug is present in relatively pure amorphous domains. The amount of CETP inhibitor present in relatively pure amorphous drug domains or regions may be determined by first preparing calibration standards of substantially homogeneous dispersions to determine T_{g} of the solid amorphous dispersion versus drug loading in the dispersion. From these calibration data and the T_{g} of the drug/polymer dispersion, the fraction of CETP inhibitor in relatively pure amorphous drug domains or regions can be determined. Alternatively, the amount of CETP inhibitor present in relatively pure amorphous drug domains or regions may be determined by comparing the magnitude of the heat capacity for the transition in the proximity of the drug T_{g} with calibration standards consisting essentially of a physical mixture of amorphous drug and polymer. In either case, a solid amorphous dispersion is considered to be substantially homogeneous if the fraction of CETP inhibitor that is present in relatively pure amorphous drug domains or regions within the solid amorphous dispersion is less than about 20 wt %, and preferably less than about 10 wt % of the total amount of CETP inhibitor.

### Concentration-Enhancing Polymers

Concentration-enhancing polymers suitable for use in the compositions of the present invention should be inert, in the sense that they do not chemically react with the CETP inhibitor in an adverse manner, are pharmaceutically acceptable, and have at least some solubility in aqueous solution at physiologically relevant pHs (e.g. 1-8). The polymer can be neutral or ionizable, and should have an aqueous-solubility of at least about 0.1 mg/mL over at least a portion of the pH range of 1-8.

Concentration-enhancing polymers suitable for use with the present invention may be cellulosic or non-cellulosic. The polymers may be neutral or ionizable in aqueous solution. Of these, ionizable and cellulosic polymers are preferred, with ionizable cellulosic polymers being more preferred.

A preferred class of polymers comprises polymers that are “amphiphilic” in nature, meaning that the polymer has hydrophobic and hydrophilic portions. The hydrophobic portion may comprise groups such as aliphatic or aromatic hydrocarbon groups. The hydrophilic portion may comprise either ionizable or non-ionizable groups that are capable of hydrogen bonding such as hydroxyls, carboxylic acids, esters, amines or amides.

Amphiphilic and/or ionizable polymers are preferred because it is believed that such polymers may tend to have relatively strong interactions with the CETP inhibitor and may promote the formation of the various types of polymer/drug assemblies in the use environment as described previously. In addition, the repulsion of the like charges of the ionized groups of such polymers may serve to limit the size of the polymer/drug assemblies to the nanometer or submicron scale. For example, while not wishing to be bound by a particular theory, such polymer/drug assemblies may comprise hydrophobic CETP inhibitor clusters surrounded by the polymer with the polymer's hydrophobic regions turned inward towards the CETP inhibitor and the hydrophilic regions of the polymer turned outward toward the aqueous environment. Alternatively, depending on the specific chemical nature of the CETP inhibitor, the ionized functional groups of the polymer may associate, for example, via ion pairing or hydrogen bonds, with ionic or polar groups of the CETP inhibitor. In the case of ionizable polymers, the hydrophilic regions of the polymer would include the ionized functional groups. Such polymer/drug assemblies in solution may well resemble charged polymeric micellar-like structures. In any case, regardless of the mechanism of action, such amphiphilic polymers, particularly ionizable cellulosic polymers, have been shown to improve the MDC and/or AUC of CETP inhibitor in aqueous solution relative to control compositions free from such polymers (described in commonly assigned U.S. patent application Ser. No. 09/918,127, filed Jul. 31, 2001, which is incorporated herein by reference).

Surprisingly, such amphiphilic polymers can greatly enhance the maximum concentration of CETP inhibitor obtained when CETP inhibitor is dosed to a use environment. In addition, such amphiphilic polymers interact with the CETP inhibitor to prevent the precipitation or crystallization of the CETP inhibitor from solution despite its concentration being substantially above its equilibrium concentration. In particular, when the preferred compositions are solid amorphous dispersions of the CETP inhibitor and the concentration-enhancing polymer, the compositions provide a greatly enhanced drug concentration, particularly when the dispersions are substantially homogeneous. The maximum drug concentration may be 10-fold and often more than 50-fold the equilibrium concentration of the crystalline CETP inhibitor. Such enhanced CETP inhibitor concentrations in turn lead to substantially enhanced relative bioavailability for the CETP inhibitor.

One class of polymers suitable for use with the present invention comprises neutral non-cellulosic polymers. Exemplary polymers include: vinyl polymers and copolymers having substituents of hydroxyl, alkylacyloxy, or cyclicamido; polyvinyl alcohols that have at least a portion of their repeat units in the unhydrolyzed (vinyl acetate) form; polyvinyl alcohol polyvinyl acetate copolymers; polyvinyl pyrrolidone; polyoxyethylene-polyoxypropylene copolymers, also known as poloxamers; and polyethylene polyvinyl alcohol copolymers.

Another class of polymers suitable for use with the present invention comprises ionizable non-cellulosic polymers. Exemplary polymers include: carboxylic acid-functionalized vinyl polymers, such as the carboxylic acid functionalized polymethacrylates and carboxylic acid functionalized polyacrylates such as the EUDRAGITS® manufactured by Rohm Tech Inc., of Malden, Mass.; amine-functionalized polyacrylates and polymethacrylates; proteins; and carboxylic acid functionalized starches such as starch glycolate.

Non-cellulosic polymers that are amphiphilic are copolymers of a relatively hydrophilic and a relatively hydrophobic monomer. Examples include acrylate and methacrylate copolymers, and polyoxyethylene-polyoxypropylene copolymers. Exemplary commercial grades of such copolymers include the EUDRAGITS, which are copolymers of methacrylates and acrylates, and the PLURONICS supplied by BASF, which are polyoxyethylene-polyoxypropylene copolymers.

A preferred class of polymers comprises ionizable and neutral cellulosic polymers with at least one ester- and/or ether-linked substituent in which the polymer has a degree of substitution of at least about 0.1 for each substituent.

It should be noted that in the polymer nomenclature used herein, ether-linked substituents are recited prior to “cellulose” as the moiety attached to the ether group; for example, “ethylbenzoic acid cellulose” has ethoxybenzoic acid substituents. Analogously, ester-linked substituents are recited after “cellulose” as the carboxylate; for example, “cellulose phthalate” has one carboxylic acid of each phthalate moiety ester-linked to the polymer and the other carboxylic acid unreacted.

It should also be noted that a polymer name such as “cellulose acetate phthalate” (CAP) refers to any of the family of cellulosic polymers that have acetate and phthalate groups attached via ester linkages to a significant fraction of the cellulosic polymer's hydroxyl groups. Generally, the degree of substitution of each substituent group can range from 0.1 to 2.9 as long as the other criteria of the polymer are met. “Degree of substitution” refers to the average number of the three hydroxyls per saccharide repeat unit on the cellulose chain that have been substituted. For example, if all of the hydroxyls on the cellulose chain have been phthalate substituted, the phthalate degree of substitution is 3. Also included within each polymer family type are cellulosic polymers that have additional substituents added in relatively small amounts that do not substantially alter the performance of the polymer.

Amphiphilic cellulosics comprise polymers in which the parent cellulosic polymer has been substituted at any or all of the 3 hydroxyl groups present on each saccharide repeat unit with at least one relatively hydrophobic substituent. Hydrophobic substituents may be essentially any substituent that, if substituted to a high enough level or degree of substitution, can render the cellulosic polymer essentially aqueous insoluble. Examples of hydrophobic substituents include ether-linked alkyl groups such as methyl, ethyl, propyl, butyl, etc.; or ester-linked alkyl groups such as acetate, propionate, butyrate, etc.; and ether- and/or ester-linked aryl groups such as phenyl, benzoate, or phenylate. Hydrophilic regions of the polymer can be either those portions that are relatively unsubstituted, since the unsubstituted hydroxyls are themselves relatively hydrophilic, or those regions that are substituted with hydrophilic substituents. Hydrophilic substituents include ether- or ester-linked nonionizable groups such as the hydroxy alkyl substituents hydroxyethyl, hydroxypropyl, and the alkyl ether groups such as ethoxyethoxy or methoxyethoxy. Particularly preferred hydrophilic substituents are those that are ether- or ester-linked ionizable groups such as carboxylic acids, thiocarboxylic acids, substituted phenoxy groups, amines, phosphates or sulfonates.

One class of cellulosic polymers comprises neutral polymers, meaning that the polymers are substantially non-ionizable in aqueous solution. Such polymers contain non-ionizable substituents, which may be either ether-linked or ester-linked. Exemplary ether-linked non-ionizable substituents include: alkyl groups, such as methyl, ethyl, propyl, butyl, etc.; hydroxy alkyl groups such as hydroxymethyl, hydroxyethyl, hydroxypropyl, etc.; and aryl groups such as phenyl. Exemplary ester-linked non-ionizable substituents include: alkyl groups, such as acetate, propionate, butyrate, etc.; and aryl groups such as phenylate. However, when aryl groups are included, the polymer may need to include a sufficient amount of a hydrophilic substituent so that the polymer has at least some water solubility at any physiologically relevant pH of from 1 to 8.

Exemplary non-ionizable polymers that may be used as the polymer include: hydroxypropyl methyl cellulose acetate, hydroxypropyl methyl cellulose, hydroxypropyl cellulose, methyl cellulose, hydroxyethyl methyl cellulose, hydroxyethyl cellulose acetate, and hydroxyethyl ethyl cellulose.

A preferred set of neutral cellulosic polymers are those that are amphiphilic. Exemplary polymers include hydroxypropyl methyl cellulose and hydroxypropyl cellulose acetate, where cellulosic repeat units that have relatively high numbers of methyl or acetate substituents relative to the unsubstituted hydroxyl or hydroxypropyl substituents constitute hydrophobic regions relative to other repeat units on the polymer. Neutral polymers suitable for use in the solid amorphous dispersions of the present invention are more fully disclosed in commonly assigned pending U.S. patent application Ser. No. 10/175,132, filed Jun. 18, 2002, herein incorporated by reference.

A preferred class of cellulosic polymers comprises polymers that are at least partially ionizable at physiologically relevant pH and include at least one ionizable substituent, which may be either ether-linked or ester-linked. Exemplary ether-linked ionizable substituents include: carboxylic acids, such as acetic acid, propionic acid, benzoic acid, salicylic acid, alkoxybenzoic acids such as ethoxybenzoic acid or propoxybenzoic acid, the various isomers of alkoxyphthalic acid such as ethoxyphthalic acid and ethoxyisophthalic acid, the various isomers of alkoxynicotinic acid such as ethoxynicotinic acid, and the various isomers of picolinic acid such as ethoxypicolinic acid, etc.; thiocarboxylic acids, such as thioacetic acid; substituted phenoxy groups, such as hydroxyphenoxy, etc.; amines, such as aminoethoxy, diethylaminoethoxy, trimethylaminoethoxy, etc.; phosphates, such as phosphate ethoxy; and sulfonates, such as sulphonate ethoxy. Exemplary ester linked ionizable substituents include: carboxylic acids, such as succinate, citrate, phthalate, terephthalate, isophthalate, trimellitate, and the various isomers of pyridinedicarboxylic acid, etc.; thiocarboxylic acids, such as thiosuccinate; substituted phenoxy groups, such as amino salicylic acid; amines, such as natural or synthetic amino acids, such as alanine or phenylalanine; phosphates, such as acetyl phosphate; and sulfonates, such as acetyl sulfonate. For aromatic-substituted polymers to also have the requisite aqueous solubility, it is also desirable that sufficient hydrophilic groups such as hydroxypropyl or carboxylic acid functional groups be attached to the polymer to render the polymer aqueous soluble at least at pH values where any ionizable groups are ionized. In some cases, the aromatic group may itself be ionizable, such as phthalate or trimellitate substituents.

Exemplary cellulosic polymers that are at least partially ionized at physiologically relevant pHs include: hydroxypropyl methyl cellulose acetate succinate, hydroxypropyl methyl cellulose succinate, hydroxypropyl cellulose acetate succinate, hydroxyethyl methyl cellulose succinate, hydroxyethyl cellulose acetate succinate, hydroxypropyl methyl cellulose phthalate, hydroxyethyl methyl cellulose acetate succinate, hydroxyethyl methyl cellulose acetate phthalate, carboxyethyl cellulose, carboxymethyl cellulose, carboxymethyl ethyl cellulose, cellulose acetate phthalate, methyl cellulose acetate phthalate, ethyl cellulose acetate phthalate, hydroxypropyl cellulose acetate phthalate, hydroxypropyl methyl cellulose acetate phthalate, hydroxypropyl cellulose acetate phthalate succinate, hydroxypropyl methyl cellulose acetate succinate phthalate, hydroxypropyl methyl cellulose succinate phthalate, cellulose propionate phthalate, hydroxypropyl cellulose butyrate phthalate, cellulose acetate trimellitate, methyl cellulose acetate trimellitate, ethyl cellulose acetate trimellitate, hydroxypropyl cellulose acetate trimellitate, hydroxypropyl methyl cellulose acetate trimellitate, hydroxypropyl cellulose acetate trimellitate succinate, cellulose propionate trimellitate, cellulose butyrate trimellitate, cellulose acetate terephthalate, cellulose acetate isophthalate, cellulose acetate pyridinedicarboxylate, salicylic acid cellulose acetate, hydroxypropyl salicylic acid cellulose acetate, ethylbenzoic acid cellulose acetate, hydroxypropyl ethylbenzoic acid cellulose acetate, ethyl phthalic acid cellulose acetate, ethyl nicotinic acid cellulose acetate, and ethyl picolinic acid cellulose acetate.

Exemplary cellulosic polymers that meet the definition of amphiphilic, having hydrophilic and hydrophobic regions include polymers such as cellulose acetate phthalate and cellulose acetate trimellitate where the cellulosic repeat units that have one or more acetate substituents are hydrophobic relative to those that have no acetate substituents or have one or more ionized phthalate or trimellitate substituents.

A particularly desirable subset of cellulosic ionizable polymers are those that possess both a carboxylic acid functional aromatic substituent and an alkylate substituent and thus are amphiphilic. Exemplary polymers include cellulose acetate phthalate, methyl cellulose acetate phthalate, ethyl cellulose acetate phthalate, hydroxypropyl cellulose acetate phthalate, hydroxypropyl methyl cellulose phthalate, hydroxypropyl methyl cellulose acetate phthalate, hydroxypropyl cellulose acetate phthalate succinate, cellulose propionate phthalate, hydroxypropyl cellulose butyrate phthalate, cellulose acetate trimellitate, methyl cellulose acetate trimellitate, ethyl cellulose acetate trimellitate, hydroxypropyl cellulose acetate trimellitate, hydroxypropyl methyl cellulose acetate trimellitate, hydroxypropyl cellulose acetate trimellitate succinate, cellulose propionate trimellitate, cellulose butyrate trimellitate, cellulose acetate terephthalate, cellulose acetate isophthalate, cellulose acetate pyridinedicarboxylate, salicylic acid cellulose acetate, hydroxypropyl salicylic acid cellulose acetate, ethylbenzoic acid cellulose acetate, hydroxypropyl ethylbenzoic acid cellulose acetate, ethyl phthalic acid cellulose acetate, ethyl nicotinic acid cellulose acetate, and ethyl picolinic acid cellulose acetate.

Another particularly desirable subset of cellulosic ionizable polymers are those that possess a non-aromatic carboxylate substituent. Exemplary polymers include hydroxypropyl methyl cellulose acetate succinate, hydroxypropyl methyl cellulose succinate, hydroxypropyl cellulose acetate succinate, hydroxyethyl methyl cellulose acetate succinate, hydroxyethyl methyl cellulose succinate, hydroxyethyl cellulose acetate succinate, and carboxymethyl ethyl cellulose.

While, as listed above, a wide range of polymers may be used to form dispersions of CETP inhibitors, the inventors have found that relatively hydrophobic polymers have shown the best performance as demonstrated by high MDC and AUC values. In particular, cellulosic polymers that are aqueous insoluble in their nonionized state but are aqueous soluble in their ionized state perform particularly well. A particular subclass of such polymers are the so-called “enteric” polymers, which include, for example, certain grades of hydroxypropyl methyl cellulose phthalate and cellulose acetate trimellitate. Dispersions formed from such polymers generally show very large enhancements, on the order of 50-fold to over 1000-fold, in the maximum drug concentration achieved in dissolution tests relative to that for a crystalline drug control. In addition, non-enteric grades of such polymers as well as closely related cellulosic polymers are expected to perform well due to the similarities in physical properties within the CETP inhibitor class.

Thus, especially preferred polymers are hydroxypropyl methyl cellulose acetate succinate (HPMCAS), hydroxypropyl methyl cellulose phthalate (HPMCP), cellulose acetate phthalate (CAP), cellulose acetate trimellitate (CAT), methyl cellulose acetate phthalate, hydroxypropyl cellulose acetate phthalate, cellulose acetate terephthalate, cellulose acetate isophthalate, and carboxymethyl ethyl cellulose. The most preferred ionizable cellulosic polymers are hydroxypropyl methyl cellulose acetate succinate, hydroxypropyl methyl cellulose phthalate, cellulose acetate phthalate, cellulose acetate trimellitate, and carboxymethyl ethyl cellulose.

One particularly effective polymer for forming dispersions of the present invention is carboxymethyl ethyl cellulose (CMEC). Dispersions made from CETP inhibitors and CMEC typically have high glass-transition temperatures at high relative humidities, due to the high glass-transition temperature of CMEC. As discussed below, such high T_{g}s result in solid amorphous dispersions with excellent physical stability. In addition, because all of the substituents on CMEC are attached to the cellulose backbone through ether linkages, CMEC has excellent chemical stability. Additionally, commercial grades of CMEC, such as that provided by Freund Industrial Company, Limited (Tokyo, Japan), are amphiphilic, leading to high degrees of concentration enhancement. Finally, hydrophobic CETP inhibitors often have a high solubility in CMEC allowing for formation of physically stable dispersions with high drug loadings.

A particularly effective concentration-enhancing polymer for use with CETP inhibitors is HPMCAS.

While specific polymers have been discussed as being suitable for use in the compositions of the present invention, blends of such polymers may also be suitable. Thus the term “polymer” is intended to include blends of polymers in addition to a single species of polymer.

To obtain the best performance, particularly upon storage for long times prior to use, it is preferred that the CETP inhibitor remain, to the extent possible, in the amorphous state. This is best achieved when the glass-transition temperature, T_{g}, of the amorphous CETP inhibitor material is substantially above the storage temperature of the composition. In particular, it is preferable that the T_{g} of the amorphous state of the CETP inhibitor be at least about 40° C. and preferably at least about 60° C. However, this is not always the case. For example, the T_{g} of amorphous torcetrapib is about 30° C. For those aspects of the invention in which the composition is a solid, substantially amorphous dispersion of a CETP inhibitor in the concentration-enhancing polymer, it is preferred that the concentration-enhancing polymer have a T_{g} of at least about 40° C., preferably at least about 70° C. and more preferably greater than 100° C. Exemplary high T_{g} polymers include HPMCAS, HPMCP, CAP, CAT, CMEC and other cellulosics that have alkylate or aromatic substituents or both alkylate and aromatic substituents.

Another preferred class of polymers consists of neutralized acidic polymers. By “neutralized acidic polymer” is meant any acidic polymer for which a significant fraction of the “acidic moieties” or “acidic substituents” have been “neutralized”; that is, exist in their deprotonated form. By “acidic polymer” is meant any polymer that possesses a significant number of acidic moieties. In general, a significant number of acidic moieties would be greater than or equal to about 0.1 milliequivalents of acidic moieties per gram of polymer. “Acidic moieties” include any functional groups that are sufficiently acidic that, in contact with or dissolved in water, can at least partially donate a hydrogen cation to water and thus increase the hydrogen-ion concentration. This definition includes any functional group or “substituent,” as it is termed when the functional group is covalently attached to a polymer that has a pKa of less than about 10. Exemplary classes of functional groups that are included in the above description include carboxylic acids, thiocarboxylic acids, phosphates, phenolic groups, and sulfonates. Such functional groups may make up the primary structure of the polymer such as for polyacrylic acid, but more generally are covalently attached to the backbone of the parent polymer and thus are termed “substituents.” Neutralized acidic polymers are described in more detail in commonly assigned copending U.S. patent application Ser. No. 10/175,566 entitled “Pharmaceutical Compositions of Drugs and Neutralized Acidic Polymers” filed Jun. 17, 2002, the relevant disclosure of which is incorporated by reference.

In addition, the preferred polymers listed above, that is amphiphilic cellulosic polymers, tend to have greater concentration-enhancing properties relative to the other polymers of the present invention. Generally those concentration-enhancing polymers that have ionizable substituents tend to perform best. In vitro tests of compositions with such polymers tend to have higher MDC and AUC values than compositions with other polymers of the invention.

### Preparation of Dispersions

The solid amorphous dispersions of CETP inhibitor and concentration-enhancing polymer may be made according to any conventional process for forming solid amorphous dispersions that results in at least a major portion (at least about 60%) of the CETP inhibitor being in the amorphous state. Such processes include mechanical, thermal and solvent processes. Exemplary mechanical processes include milling and extrusion; melt processes including high temperature fusion, solvent-modified fusion and melt-congeal processes; and solvent processes including non-solvent precipitation, spray-coating and spray-drying. See, for example, the following U.S. patents, the pertinent disclosures of which are incorporated herein by reference: Nos. 5,456,923 and 5,939,099, which describe forming dispersions by extrusion processes; Nos. 5,340,591 and 4,673,564, which describe forming dispersions by milling processes; and Nos. 5,707,646 and 4,894,235, which describe forming dispersions by melt congeal processes.

When the CETP inhibitor has a relatively low melting point, typically less than about 200° C. and preferably less than about 150° C., the use of a melt-congeal or melt-extrusion process is advantageous. In such processes, a molten mixture comprising the CETP inhibitor and concentration-enhancing polymer is rapidly cooled to solidify the molten mixture to form a solid amorphous dispersion. By “molten mixture” is meant that the mixture comprising the CETP inhibitor and concentration-enhancing polymer is heated sufficiently that it becomes sufficiently fluid that the CETP inhibitor substantially disperses in one or more of the concentration-enhancing polymers and other excipients. Generally, this requires that the mixture be heated to about 10° C. or more above the melting point of the lowest melting excipient or CETP inhibitor in the composition. The CETP inhibitor may exist in the molten mixture as a pure phase, as a solution of CETP inhibitor homogeneously distributed throughout the molten mixture, or any combination of these states or those states that lie intermediate between them. The molten mixture is preferably substantially homogeneous so that the CETP inhibitor is dispersed as homogeneously as possible throughout the molten mixture. When the temperature of the molten mixture is below the melting point of both the CETP inhibitor and the concentration-enhancing polymer, the molten excipients, concentration-enhancing polymer, and CETP inhibitor are preferably sufficiently soluble in each other that a substantial portion of the CETP inhibitor disperses in the concentration-enhancing polymer or excipients. It is often preferred that the mixture be heated above the lower of the melting points of the concentration-enhancing polymer and the CETP inhibitor. It should be noted that many concentration-enhancing polymers are amorphous. In such cases, melting point refers to the softening point of the polymer. Thus, although the term “melting point” generally refers specifically to the temperature at which a crystalline material transitions from its crystalline to its liquid state, as used herein, the term is used more broadly, referring to the heating of any material or mixture of materials sufficiently that it becomes fluid in a manner similar to a crystalline material in the fluid state.

Generally, the processing temperature may vary from 50° C. up to about 200° C. or higher, depending on the melting point of the CETP inhibitor and polymer, the latter being a function of the polymer grade selected. However, the processing temperature should not be so high that an unacceptable level of degradation of the CETP inhibitor or polymer occurs. In some cases, the molten mixture should be formed under an inert atmosphere to prevent degradation of the CETP inhibitor and/or polymer at the processing temperature. When relatively high temperatures are used, it is often preferable to minimize the time that the mixture is at the elevated temperature to minimize degradation.

The molten mixture may also include an excipient that will reduce the melting temperature of the molten mixture, thereby allowing processing at a lower temperature. When such excipients have low volatility and substantially remain in the mixture upon solidification, they generally can comprise up to 30 wt % of the molten mixture. For example, a plasticizer may be added to the mixture to reduce the melting temperature of the polymer. Examples of plasticizers include water, triethylcitrate, triacetin, and dibutyl sebacate. Volatile agents that dissolve or swell the polymer, such as acetone, water, methanol and ethyl acetate, may also be added to reduce the melting point of the molten mixture. When such volatile excipients are added, at least a portion, up to essentially all of such excipients may evaporate in the process of or following conversion of the molten mixture to a solid mixture. In such cases, the processing may be considered to be a combination of solvent processing and melt-congealing or melt-extrusion. Removal of such volatile excipients from the molten mixture can be accomplished by breaking up or atomizing the molten mixture into small droplets and contacting the droplets with a fluid so that the droplets both cool and lose all or part of the volatile excipient. Examples of other excipients that can be added to the mixture to reduce the processing temperature include low molecular weight polymers or oligomers, such as polyethylene glycol, polyvinylpyrrolidone, and poloxamers; fats and oils, including mono-, di-, and triglycerides; natural and synthetic waxes, such as carnauba wax, beeswax, microcrystalline wax, castor wax, and paraffin wax; long chain alcohols, such as cetyl alcohol and stearyl alcohol; and long chain fatty acids, such as stearic acid. As mentioned above, when the excipient added is volatile, it may be removed from the mixture while still molten or following solidification to form the solid amorphous dispersion.

Virtually any process may be used to form the molten mixture. One method involves melting the concentration-enhancing polymer in a vessel and then adding the CETP inhibitor to the molten polymer. Another method involves melting the CETP inhibitor in a vessel and then adding the concentration-enhancing polymer. In yet another method, a solid blend of the CETP inhibitor and concentration-enhancing polymer may be added to a vessel and the blend heated to form the molten mixture.

Once the molten mixture is formed, it may be mixed to ensure the CETP inhibitor is homogeneously distributed throughout the molten mixture. Such mixing may be done using mechanical means, such as overhead mixers, magnetically driven mixers and stir bars, planetary mixers, and homogenizers. Optionally, when the molten mixture is formed in a vessel, the contents of the vessel can be pumped out of the vessel and through an in-line or static mixer and then returned to the vessel. The amount of shear used to mix the molten mixture should be sufficiently high to ensure uniform distribution of the CETP inhibitor in the molten mixture. The molten mixture can be mixed from a few minutes to several hours, the mixing time depending on the viscosity of the mixture and the solubility of the CETP inhibitor and the presence of optional excipients in the concentration-enhancing polymer.

Yet another method of preparing the molten mixture is to use two vessels, melting the CETP inhibitor in the first vessel and the concentration-enhancing polymer in a second vessel. The two melts are then pumped through an in-line static mixer or extruder to produce the molten mixture that is then rapidly solidified.

Still another method of preparing the molten mixture is by the use of an extruder, such as a single-screw or twin-screw extruder, both well known in the art. In such devices, a solid feed of the composition is fed to the extruder, whereby the combination of heat and shear forces produce a uniformly mixed molten mixture, which can then be rapidly solidified to form the solid amorphous dispersion. The solid feed can be prepared using methods well known in the art for obtaining solid mixtures with high content uniformity. Alternatively, the extruder may be equipped with two feeders, allowing the CETP inhibitor to be fed to the extruder through one feeder and the polymer through the other. Other excipients to reduce the processing temperature as described above may be included in the solid feed, or in the case of liquid excipients, such as water, may be injected into the extruder using methods well known in the art.

The extruder should be designed so that it produces a molten mixture with the CETP inhibitor uniformly distributed throughout the composition. Various zones in the extruder should be heated to appropriate temperatures to obtain the desired extrudate temperature as well as the desired degree of mixing or shear, using procedures well known in the art.

When the CETP inhibitor has a high solubility in the concentration-enhancing polymer, a lower amount of mechanical energy will be required to form the solid amorphous dispersion. In the case where the melting point of the undispersed CETP inhibitor is greater than the melting point of the undispersed concentration-enhancing polymer, the processing temperature may be below the melting temperature of the undispersed CETP inhibitor but greater than the melting point of the polymer, since the CETP inhibitor will dissolve into the molten polymer. When the melting point of the undispersed CETP inhibitor is less than the melting point of the undispersed concentration-enhancing polymer, the processing temperature may be above the melting point of the undispersed CETP inhibitor but below the melting point of the undispersed concentration-enhancing polymer since the molten CETP inhibitor will dissolve in or be absorbed into the polymer.

When the CETP inhibitor has a low solubility in the polymer, a higher amount of mechanical energy may be required to form the solid amorphous dispersion. Here, the processing temperature may need to be above the melting point of the CETP inhibitor and the polymer. As mentioned above, alternatively, a liquid or low-melting point excipient may be added that promotes melting or the mutual solubility of the concentration-enhancing polymer and a CETP inhibitor. A high amount of mechanical energy may also be needed to mix the CETP inhibitor and the polymer to form a dispersion. Typically, the lowest processing temperature and an extruder design that imparts the lowest amount of mechanical energy, i.e., shear, that produces a satisfactory dispersion (substantially amorphous and substantially homogeneous) is chosen in order to minimize the exposure of the CETP inhibitor to harsh conditions.

Once the molten mixture of CETP inhibitor and concentration-enhancing polymer is formed, the mixture should be rapidly solidified to form the solid amorphous dispersion. By “rapidly solidified” is meant that the molten mixture is solidified sufficiently fast that substantial phase separation of the CETP inhibitor and polymer does not occur. Typically, this means that the mixture should be solidified in less than about 10 minutes, preferably less than about 5 minutes and more preferably less than about 1 minute. If the mixture is not rapidly solidified, phase separation can occur, resulting in the formation of CETP inhibitor-rich and polymer-rich phases.

Solidification often takes place primarily by cooling the molten mixture to at least about 10° C. and preferably at least about 30° C. below it's melting point. As mentioned above, solidification can be additionally promoted by evaporation of all or part of one or more volatile excipients or solvents. To promote rapid cooling and evaporation of volatile excipients, the molten mixture is often formed into a high surface area shape such as a rod or fiber or droplets. For example, the molten mixture can be forced through one or more small holes to form long thin fibers or rods or may be fed to a device, such as an atomizer such as a rotating disk, that breaks the molten mixture up into droplets from 1 μm to 1 cm in diameter. The droplets are then contacted with a relatively cool fluid such as air or nitrogen to promote cooling and evaporation.

A useful tool for evaluating and selecting conditions for forming substantially homogeneous, substantially amorphous dispersions via a melt-congeal or melt-extrusion process is the differential scanning calorimeter (DSC). While the rate at which samples can be heated and cooled in a DSC is limited, it does allow for precise control of the thermal history of a sample. For example, the CETP inhibitor and concentration-enhancing polymer may be dry-blended and then placed into the DSC sample pan. The DSC can then be programmed to heat the sample at the desired rate, hold the sample at the desired temperature for a desired time, and then rapidly cool the sample to ambient or lower temperature. The sample can then be re-analyzed on the DSC to verify that it was transformed into a substantially homogeneous, substantially amorphous dispersion (i.e., the sample has a single Tg). Using this procedure, the temperature and time required to achieve a substantially homogeneous, substantially amorphous dispersion for a given CETP inhibitor and concentration-enhancing polymer can be determined.

Another method for forming solid amorphous dispersions is by “solvent processing,” which consists of dissolution of the CETP inhibitor and one or more polymers in a common solvent. “Common” here means that the solvent, which can be a mixture of compounds, will dissolve both the CETP inhibitor and the polymer(s). After both the CETP inhibitor and the polymer have been dissolved, the solvent is rapidly removed by evaporation or by mixing with a non-solvent. Exemplary processes are spray-drying, spray-coating (pan-coating, fluidized bed coating, etc.), and precipitation by rapid mixing of the polymer and CETP inhibitor solution with CO₂, water, or some other non-solvent. Preferably, removal of the solvent results in the formation of a substantially homogeneous, solid amorphous dispersion. In such dispersions, the CETP inhibitor is dispersed as homogeneously as possible throughout the polymer and can be thought of as a solid solution of CETP inhibitor dispersed in the polymer(s), wherein the solid amorphous dispersion is thermodynamically stable, meaning that the concentration of CETP inhibitor in the polymer is at or below its equilibrium value, or it may be considered to be a supersaturated solid solution where the CETP inhibitor concentration in the concentration-enhancing polymer(s) is above its equilibrium value.

The solvent may be removed by spray-drying. The term “spray-drying” is used conventionally and broadly refers to processes involving breaking up liquid mixtures into small droplets (atomization) and rapidly removing solvent from the mixture in a spray-drying apparatus where there is a strong driving force for evaporation of solvent from the droplets. Spray-drying processes and spray-drying equipment are described generally in Perry's Chemical Engineers' Handbook, pages 20-54 to 20-57 (Sixth Edition 1984). More details on spray-drying processes and equipment are reviewed by Marshall, “Atomization and Spray-Drying,” 50 Chem. Eng. Prog. Monogr. Series 2 (1954), and Masters, Spray Drying Handbook (Fourth Edition 1985). The strong driving force for solvent evaporation is generally provided by maintaining the partial pressure of solvent in the spray-drying apparatus well below the vapor pressure of the solvent at the temperature of the drying droplets. This is accomplished by (1) maintaining the pressure in the spray-drying apparatus at a partial vacuum (e.g., 0.01 to 0.50 atm); or (2) mixing the liquid droplets with a warm drying gas; or (3) both (1) and (2). In addition, at least a portion of the heat required for evaporation of solvent may be provided by heating the spray solution.

Solvents suitable for spray-drying can be any organic compound in which the CETP inhibitor and polymer are mutually soluble. Preferably, the solvent is also volatile with a boiling point of 150° C. or less. In addition, the solvent should have relatively low toxicity and be removed from the solid amorphous dispersion to a level that is acceptable according to The International Committee on Harmonization (ICH) guidelines. Removal of solvent to this level may require a subsequent processing step such as tray-drying. Preferred solvents include alcohols such as methanol, ethanol, n-propanol, iso-propanol, and butanol; ketones such as acetone, methyl ethyl ketone and methyl iso-butyl ketone; esters such as ethyl acetate and propylacetate; and various other solvents such as acetonitrile, methylene chloride, toluene, and 1,1,1-trichloroethane. Lower volatility solvents such as dimethyl acetamide or dimethylsulfoxide can also be used. Mixtures of solvents, such as 50% methanol and 50% acetone, can also be used, as can mixtures with water, so long as the polymer and CETP inhibitor are sufficiently soluble to make the spray-drying process practicable. Generally, due to the hydrophobic nature of low-solubility CETP inhibitors, non-aqueous solvents are preferred, meaning that the solvent comprises less than about 10 wt % water.

The solvent-bearing feed, comprising the CETP inhibitor and the concentration-enhancing polymer, can be spray-dried under a wide variety of conditions and yet still yield dispersions with acceptable properties. For example, various types of nozzles can be used to atomize the spray solution, thereby introducing the spray solution into the spray-dry chamber as a collection of small droplets. Essentially any type of nozzle may be used to spray the solution as long as the droplets that are formed are sufficiently small that they dry sufficiently (due to evaporation of solvent) that they do not stick to or coat the spray-drying chamber wall.

Although the maximum droplet size varies widely as a function of the size, shape and flow pattern within the spray-dryer, generally droplets should be less than about 500 μm in diameter when they exit the nozzle. Examples of types of nozzles that may be used to form the solid amorphous dispersions include the two-fluid nozzle, the fountain-type nozzle, the flat fan-type nozzle, the pressure nozzle and the rotary atomizer. In a preferred embodiment, a pressure nozzle is used, as disclosed in detail in commonly assigned copending U.S. Provisional Application No. 60/353,986, the disclosure of which is incorporated herein by reference.

The spray solution can be delivered to the spray nozzle or nozzles at a wide range of temperatures and flow rates. Generally, the spray solution temperature can range anywhere from just above the solvent's freezing point to about 20° C. above its ambient pressure boiling point (by pressurizing the solution) and in some cases even higher. Spray solution flow rates to the spray nozzle can vary over a wide range depending on the type of nozzle, spray-dryer size and spray-dry conditions such as the inlet temperature and flow rate of the drying gas. Generally, the energy for evaporation of solvent from the spray solution in a spray-drying process comes primarily from the drying gas.

The drying gas can, in principle, be essentially any gas, but for safety reasons and to minimize undesirable oxidation of the CETP inhibitor or other materials in the solid amorphous dispersion, an inert gas such as nitrogen, nitrogen-enriched air or argon is utilized. The drying gas is typically introduced into the drying chamber at a temperature between about 60° and about 300° C. and preferably between about 80° and about 240° C.

The large surface-to-volume ratio of the droplets and the large driving force for evaporation of solvent leads to rapid solidification times for the droplets. Solidification times should be less than about 20 seconds, preferably less than about 10 seconds, and more preferably less than 1 second. This rapid solidification is often critical to the particles maintaining a uniform, homogeneous dispersion instead of separating into CETP inhibitor-rich and polymer-rich phases. In a preferred embodiment, the height and volume of the spray-dryer are adjusted to provide sufficient time for the droplets to dry prior to impinging on an internal surface of the spray-dryer, as described in detail in commonly assigned, copending U.S. Provisional Application No. 60/354,080, incorporated herein by reference. As noted above, to get large enhancements in concentration and bioavailability it is often necessary to obtain as homogeneous a dispersion as possible.

Following solidification, the solid powder typically stays in the spray-drying chamber for about 5 to 60 seconds, further evaporating solvent from the solid powder. The final solvent content of the solid dispersion as it exits the dryer should be low, since this reduces the mobility of the CETP inhibitor molecules in the solid amorphous dispersion, thereby improving its stability. Generally, the solvent content of the solid amorphous dispersion as it leaves the spray-drying chamber should be less than about 10 wt % and preferably less than about 2 wt %. Following formation, the solid amorphous dispersion can be dried to remove residual solvent using suitable drying processes, such as tray drying, fluid bed drying, microwave drying, belt drying, rotary drying, and other drying processes known in the art.

The solid amorphous dispersion is usually in the form of small particles. The mean size of the particles may be less than about 500 μm in diameter, or less than about 100 μm in diameter, less than about 50 μm in diameter or less than about 25 μm in diameter. When the solid amorphous dispersion is formed by spray-drying, the resulting dispersion is in the form of such small particles. When the solid amorphous dispersion is formed by other methods such by melt-congeal or extrusion processes, the resulting dispersion may be sieved, ground, or otherwise processed to yield a plurality of small particles.

Once the solid amorphous dispersion comprising the CETP inhibitor and concentration-enhancing polymer has been formed, several processing operations can be used to facilitate incorporation of the dispersion into a dosage form. These processing operations include drying, granulation, and milling.

The solid amorphous dispersion may be granulated to increase particle size and improve handling of the dispersion while forming a suitable dosage form. Preferably, the average size of the granules will range from 50 to 1000 μm. Such granulation processes may be performed before or after the composition is dried, as described above. Dry or wet granulation processes can be used for this purpose. An example of a dry granulation process is roller compaction. Wet granulation processes can include so-called low shear and high shear granulation, as well as fluid bed granulation. In these processes, a granulation fluid is mixed with the composition after the dry components have been blended to aid in the formation of the granulated composition. Examples of granulation fluids include water, ethanol, isopropyl alcohol, n-propanol, the various isomers of butanol, and mixtures thereof.

If a wet granulation process is used, the granulated composition is often dried prior to further processing. Examples of suitable drying processes to be used in connection with wet granulation are the same as those described above. Where the solid amorphous dispersion is made by a solvent process, the composition can be granulated prior to removal of residual solvent. During the drying process, residual solvent and granulation fluid are concurrently removed from the composition.

Once the composition has been granulated, it may then be milled to achieve the desired particle size. Examples of suitable processes for milling the composition include hammer milling, ball milling, fluid-energy milling, roller milling, cutting milling, and other milling processes known in the art.

Processes for forming solid amorphous dispersions of CETP inhibitors and concentration-enhancing polymers are described in detail in commonly assigned, copending U.S. patent application Ser. Nos. 09/918,127 and 10/066,091, incorporated herein by reference.

The solid amorphous dispersions of CETP inhibitors may be formulated into a controlled-release device using the methods outlined above.

### Lipid Vehicle Formulations

In a separate aspect of the invention, the CETP inhibitor in a solubility-improved form comprises a CETP inhibitor and a lipophilic vehicle selected from a digestible oil, a lipophilic solvent (also referred to herein as a “cosolvent”, whether or not another solvent is in fact present), a lipophilic surfactant, and mixtures of any two or more thereof. Embodiments include a CETP inhibitor and: (1) the combination of a pharmaceutically acceptable digestible oil and a surfactant; (2) the combination of a pharmaceutically acceptable digestible oil and a lipophilic solvent that is miscible therewith; and (3) the combination of a pharmaceutically acceptable digestible oil, a lipophilic solvent, and a surfactant.

In one embodiment, the invention provides a composition of matter for increasing the oral bioavailability of a CETP inhibitor. The composition comprises:

1. a CETP inhibitor;
2. a cosolvent;
3. a surfactant having an HLB of from 1 to not more than about 8;
4. a surfactant having an HLB of over 8 up to 20; and
5. optionally, a digestible oil.

In such formulations, all of the excipients are pharmaceutically acceptable. The above composition is sometimes referred to herein as a “pre-concentrate”, in reference to its function of forming a stable emulsion when gently mixed with water or other aqueous medium, usually gastrointestinal fluids. It is also referred to herein as a “fill”, referring to its utility as a fill for a softgel capsule.

Reference herein is frequently made to a softgel as a preferred dosage form for use with this invention, “softgel” being an abbreviation for soft gelatin capsules. It is understood that when reference is made to the term “softgel” alone, it shall be understood that the invention applies equally to all types of gelatin and non-gelatin capsules, regardless of hardness, softness, and so forth.

A cosolvent means a solvent in which the CETP inhibitor of interest is highly soluble, having, for any given CETP inhibitor, a solubility of at least about 150 mg/mL.

As noted above, and as discussed further below, a digestible oil can form a part of the pre-concentrate. If no other component of the pre-concentrate is capable of functioning as an emulsifiable oily phase, a digestible oil can be included as the oil which acts as a solvent for the CETP inhibitor and which disperses to form the (emulsifiable) oil droplet phase once the pre-concentrate has been added to water. Some surfactants can serve a dual function, however, i.e., that of acting as a surfactant and also as a solvent and an oily vehicle for forming an oil-in-water emulsion. In the event such a surfactant is employed, and, depending on the amount used, a digestible oil may be required in less of an amount, or not required at all.

The pre-concentrate can be self-emulsifying or self-microemulsifying.

The term “self-emulsifying” refers to a formulation which, when diluted by a factor of at least about 100 by water or other aqueous medium and gently mixed, yields an opaque, stable oil/water emulsion with a mean droplet diameter less than about 5 microns, but greater than about 100 nm, and which is generally polydisperse. Such an emulsion is stable for at least several (i.e., for at least about 6) hours, meaning there is no visibly detectable phase separation and that there is no visibly detectable crystallization of CETP inhibitor.

The term “self-microemulsifying” refers to a pre-concentrate which, upon at least about 100× dilution with an aqueous medium and gentle mixing, yields a non-opaque, stable oil/water emulsion with an average droplet size of about 1 micron or less, said average particle size preferably being less than about 100 nm. The particle size is primarily unimodal. Most preferably the emulsion is transparent and has a unimodal particle size distribution with a mean diameter less than about 50 nm as determined, for example, by dynamic light scattering. The microemulsion is thermodynamically stable and without any indication of crystallization of CETP inhibitor.

“Gentle mixing” as used above is understood in the art to refer to the formation of an emulsion by gentle hand (or machine) mixing, such as by repeated inversions on a standard laboratory mixing machine. High shear mixing is not required to form the emulsion. Such pre-concentrates generally emulsify nearly spontaneously when introduced into the human (or other animal) gastrointestinal tract.

Combinations of 2 surfactants, one being a low HLB surfactant with an HLB of 1 to 8, the other being a high HLB surfactant with a higher HLB of over 8 to 20, preferably 9 to 20, can be employed to create the right conditions for efficient emulsification. The HLB, an acronym for “hydrophobic-lipophilic balance”, is a rating scale that can range from 1-20 for non-ionic surfactants. The higher the HLB, the more hydrophilic the surfactant. Hydrophilic surfactants (HLB ca. 8-20), when used alone, provide fine emulsions which are, advantageously, more likely to empty uniformly from the stomach and provide a much higher surface area for absorption. Disadvantageously, however, limited miscibility of such high HLB surfactants with oils can limit their effectiveness, and thus a low HLB, lipophilic surfactant (HLB ca. 1-8) is also included. This combination of surfactants can also provide superior emulsification. A combination of a medium chain triglyceride (such as Miglyol® 812), Polysorbate 80 (HLB 15) and medium chain mono/diglycerides (Capmul® MCM, HLB=6) was found to be as efficient as Miglyol® 812 and a surfactant with an HLB of 10 (Labrafac® CM). N. H. Shah et al. Int. J. Pharm., vol 106, 15 (1994). The advantages of using combinations of high and low HLB surfactants for self-emulsifying systems, including promotion of lipolysis, have been demonstrated by Lacy, U.S. Pat. No. 6,096,338.

Suitable digestible oils, which can be used alone as the vehicle or in a vehicle that includes a digestible oil as part of a mixture, include medium chain triglycerides (MCT, C6-C12) and long chain triglycerides (LCT, C14-C20) and mixtures of mono-, di-, and triglycerides, or lipophilic derivatives of fatty acids such as esters with alkyl alcohols. Examples of preferred MCT's include fractionated coconut oils, such as Miglyol® 812, which is a 56% caprylic (C8) and 36% capric (C10) triglyceride, Miglyol® 810 (68% C8 and 28% C10), Neobee® M5, Captex® 300, Captex® 355, and Crodamol® GTCC. The Miglyols are supplied by Condea Vista Inc. (Huls), Neobee® by Stepan Europe, Voreppe, France, Captex® by Abitec Corp., and Crodamol® by Croda Corp. Examples of LCTs include vegetable oils such as soybean, safflower, corn, olive, cottonseed, arachis, sunflower seed, palm, or rapeseed. Examples of fatty acid esters of alkyl alcohols include ethyl oleate and glyceryl monooleate. Of the digestible oils MCT's are preferred, and Miglyol® 812 is most preferred.

The vehicle may also be a pharmaceutically acceptable solvent, for use alone, or as a cosolvent in a mixture. Suitable solvents include any solvent that is used to increase solubility of the CETP inhibitor in the formulation in order to allow delivery of the desired dose per dosing unit. It is not generally possible to predict the solubility of CETP inhibitors in the individual solvents, but such can be easily determined by “trial runs”. Suitable solvents include triacetin (1,2,3-propanetriyl triacetate or glyceryl triacetate available from Eastman Chemical Corp.) or other polyol esters of fatty acids, trialkyl citrate esters, propylene carbonate, dimethylisosorbide, ethyl lactate, N-methylpyrrolidones, transcutol, glycofurol, peppermint oil, 1,2-propylene glycol, ethanol, and polyethylene glycols. Preferred as solvents are triacetin, propylene carbonate (Huntsman Corp.), transcutol (Gattefosse), ethyl lactate (Purac, Lincolnshire, Nebr.) and dimethylisosorbide (sold under the registered trademark ARLASOLVE DMI, ICI Americas). A hydrophilic solvent is more likely to migrate to the capsule shell and soften the shell, and, if volatile, its concentration in the composition can be reduced, but with a potential negative impact on active component (CETP inhibitor) solubility. More preferred are the lipophilic solvents triacetin, ethyl lactate and propylene carbonate.

Hydrophilic surfactants having an HLB of 8-20, preferably having an HLB greater than about 10, are particularly effective at reducing emulsion droplet particle size. Suitable choices include nonionic surfactants such as polyoxyethylene 20 sorbitan monooleate, polysorbate 80, sold under the trademark TWEEN 80, available commercially from ICI; polyoxyethylene 20 sorbitan monolaurate (Polysorbate 20, TWEEN 20); polyethylene (40 or 60) hydrogenated castor oil (available under the registered trademarks CREMOPHOR® RH40 and RH60 from BASF); polyoxyethylene (35) castor oil (CREMOPHOR® EL); polyethylene (60) hydrogenated castor oil (Nikkole HCO-60); alpha tocopheryl polyethylene glycol 1000 succinate (Vitamin E TPGS); glyceryl PEG 8 caprylate/caprate (available commercially under the registered trademark LABRASOL® from Gattefosse); PEG 32 glyceryl laurate (sold commercially under the registered trademark GELUCIRE® 44/14 by Gattefosse), polyoxyethylene fatty acid esters (available commercially under the registered trademark MYRJ from ICI), polyoxyethylene fatty acid ethers (available commercially under the registered trademark BRIJ from ICI). Preferred are Polysorbate 80, CREMOPHOR® RH40 (BASF), and Vitamin E TPGS (Eastman).

Lipophilic surfactants having an HLB of less than about 8 are useful for achieving a balance of polarity to provide a stable emulsion, and have also been used to reverse the lipolysis inhibitory effect of hydrophilic surfactants. Suitable lipophilic surfactants include mono and diglycerides of capric and caprylic acid under the following registered trademarks: Capmul® MCM, MCM 8, and MCM 10, available commercially from Abitec; and Imwitor® 988, 742 or 308, available commercially from Condea Vista; polyoxyethylene 6 apricot kernel oil, available under the registered trademark Labrafil® M 1944 CS from Gattefosse; polyoxyethylene corn oil, available commercially as Labrafil® M 2125; propylene glycol monolaurate, available commercially as Lauroglycol from Gattefosse; propylene glycol dicaprylate/caprate available commercially as Captex® 200 from Abitec or Miglyol® 840 from Condea Vista, polyglyceryl oleate available commercially as Plurol oleique from Gattefosse, sorbitan esters of fatty acids (e.g. Span® 20, Crill® 1, Crill® 4, available commercially from ICI and Croda), and glyceryl monooleate (Maisine, Peceol). Preferred from this class are Capmul® MCM (Abitec Corp.) and Labrafil® M1944 CS (Gattefosse).

In addition to the main liquid formulation ingredients previously noted, other stabilizing additives, as conventionally known in the art of softgel formulation, can be introduced to the fill as needed, usually in relatively small quantities, such as antioxidants (BHA, BHT, tocopherol, propyl gallate, etc.) and other preservatives such as benzyl alcohol or parabens.

The composition can be formulated as a fill encapsulated in a soft gelatin capsule, a hard gelatin capsule with an appropriate seal, a non-gelatin capsule such as a hydroxypropyl methylcellulose capsule or an oral liquid or emulsion by methods commonly employed in the art. The fill is prepared by mixing the excipients and CETP inhibitor with heating if required.

The ratio of CETP inhibitor, digestible oil, cosolvent, and surfactants depends upon the efficiency of emulsification and the solubility, and the solubility depends on the dose per capsule that is desired. A self-emulsifying formulation is generally useful if the primary goals are to deliver a high dose per softgel (at least about 60 mg) with, generally, a much lower food effect than with an oil solution alone. In general, softgel preconcentrates having solubilities of CETP inhibitor of at least about 140 mg/mL in the preconcentrate, and thus requiring higher amounts of cosolvent and lower levels of surfactants and oil, are preferred.

In general, the following ranges, in weight percent, of the components for a self-emulsifying formulation of CETP inhibitors are:

1-50% CETP inhibitor

5-60% cosolvent

5-75% high HLB surfactant

5-75% low HLB surfactant

Preferred ranges that have advantageously low food effects include those stated immediately below:
1-33% CETP inhibitor
0-30% digestible oil
15-55% cosolvent
5-40% high HLB surfactant
10-50% low HLB surfactant

General ranges, in weight percent, for the components for a self-microemulsifying formulation of CETP inhibitors are

1-40% CETP inhibitor

5-65% digestible oil

5-60% cosolvent

10-75% high HLB surfactant

5-75% low HLB surfactant

Further details of such lipid vehicle formulations are disclosed in commonly assigned copending U.S. patent application Ser. No. 10/175,643 filed on Jun. 19, 2002, which is incorporated in its entirety by reference.

Such lipid vehicle formulations can be formulated into controlled and immediate release devices, such as those described above.

### HMG-CoA Reductase Inhibitors

The HMG-CoA reductase inhibitor may be any HMG-CoA reductase inhibitor capable of lowering plasma concentrations of low-density lipoprotein, total cholesterol, or both. In one aspect, the HMG-CoA reductase inhibitor is from a class of therapeutics commonly called statins. Examples of HMG-CoA reductase inhibitors that may be used include but are not limited to lovastatin (MEVACOR®); see U.S. Pat. Nos. 4,231,938; 4,294,926; 4,319,039), simvastatin (ZOCOR®; see U.S. Pat. Nos. 4,444,784; 4,450,171, 4,820,850; 4,916,239), pravastatin (PRAVACHOL®; see U.S. Pat. Nos. 4,346,227; 4,537,859; 4,410,629; 5,030,447 and 5,180,589), lactones of pravastatin (see U.S. Pat. No. 4,448,979), fluvastatin (LESCOL®; see U.S. Pat. Nos. 5,354,772; 4,911,165; 4,739,073; 4,929,437; 5,189,164; 5,118,853; 5,290,946; 5,356,896), lactones of fluvastatin, atorvastatin (LIPITOR®; see U.S. Pat. Nos. 5,273,995; 4,681,893; 5,489,691; 5,342,952), lactones of atorvastatin, rosuvastatin (Crestor®; see U.S. Pat. Nos. 5,260,440 and RE37314, and European Patent No. EP521471), lactones of rosuvastatin, itavastatin, nisvastatin, visastatin, atavastatin, bervastatin, compactin, dihydrocompactin, dalvastatin, fluindostatin, pitivastatin, mevastatin (see U.S. Pat. No. 3,983,140), and velostatin (also referred to as synvinolin). Other examples of HMG-CoA reductase inhibitors are described in U.S. Pat. Nos. 5,217,992; 5,196,440; 5,189,180; 5,166,364; 5,157,134; 5,110,940; 5,106,992; 5,099,035; 5,081,136; 5,049,696; 5,049,577; 5,025,017; 5,011,947; 5,010,105; 4,970,221; 4,940,800; 4,866,058; 4,686,237; 4,647,576; European Application Nos. 0142146A2 and 0221025A1; and PCT Application Nos. WO 86/03488 and WO 86/07054. Also included are pharmaceutically acceptable forms of the above. All of the above references are incorporated herein by reference. Preferably the HMG-CoA reductase inhibitor is selected from the group consisting of fluvastatin, lovastatin, pravastatin, atorvastatin, simvastatin, rivastatin, mevastatin, velostatin, compactin, dalvastatin, fluindostatin, rosuvastatin, pitivastatin, dihydrocompactin, and pharmaceutically acceptable forms thereof. By “pharmaceutically acceptable forms” is meant any pharmaceutically acceptable derivative or variation, including stereoisomers, stereoisomer mixtures, enantiomers, solvates, hydrates, isomorphs, pseudomorphs, polymorphs, salt forms and prodrugs.

In one embodiment, the HMG-CoA reductase inhibitor is selected from the group consisting of trans-6-[2-(3 or 4-carboxamido-substituted pyrrol-1-yl)alkyl]-4-hydroxypyran-2-ones and corresponding pyran ring-opened hydroxy acids derived therefrom. These compounds have been described in U.S. Pat. No. 4,681,893, which is herewith incorporated by reference in the present specification. The pyran ring-opened hydroxy acids that are intermediates in the synthesis of the lactone compounds can be used as free acids or as pharmaceutically acceptable metal or amine salts. In particular, these compounds can be represented by the following structure:

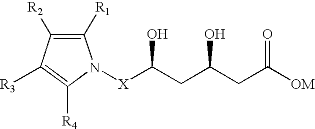

wherein X is —CH₂—, —CH₂CH₂—, —CH₂CH₂CH₂— or —CH₂CH(CH₃)—;
R₁ is 1-naphthyl; 2-naphthyl; cyclohexyl, norbornenyl; 2-, 3-, or 4-pyridinyl; phenyl; phenyl substituted with fluorine, chlorine, bromine, hydroxyl, trifluoromethyl, alkyl of from one to four carbon atoms, alkoxy of from one to four carbon atoms, or alkanoylalkoxy of from two to eight carbon atoms; either R₂ or R₃ is —CONR₅ R₆ where R₅ and R₆ are independently hydrogen; alkyl of from one to six carbon atoms; 2-, 3-, or 4-pyridinyl; phenyl; phenyl substituted with fluorine, chlorine, bromine, cyano, trifluoromethyl, or carboalkoxy of from three to eight carbon atoms; and the other of R₂ or R₃ is hydrogen; alkyl of from one to six carbon atoms; cyclopropyl; cyclobutyl; cyclopentyl; cyclohexyl; phenyl; or phenyl substituted with fluorine, chlorine, bromine, hydroxyl, trifluoromethyl, alkyl of from one to four carbon atoms, alkoxy of from one to four carbon atoms, or alkanoyloxy of from two to eight carbon atoms; R₄ is alkyl of from one to six carbon atoms; cyclopropyl; cyclobutyl; cyclopentyl; cyclohexyl; or trifluoromethyl; and M is a pharmaceutically acceptable salt (e.g., counter ion), which includes a pharmaceutically acceptable metal salt or a pharmaceutically acceptable amine salt.

Among the stereo-specific isomers, one preferred HMG-CoA reductase inhibitor is atorvastatin trihydrate hemi-calcium salt. This preferred compound is the ring-opened form of (2R-trans)-5-(4-fluorophenyl)-2-(1 methylethyl)-N,4-diphenyl-1-[2-(tetrahydro-4-hydroxy-6-oxo-2H-pyran-2-yl)ethyl]-1H-pyrrole-3-carboxamide, namely, the enantiomer [R—(R*, R*)]-2-(4-fluorophenyl-β, δ̅dihydroxy-5-(1-methylethyl)-3-phenyl-4-[(phenylamino)carbonyl)]-1H-pyrrole-1-heptanoic acid hemicalcium salt. Its chemical structure may be represented by the following structure:

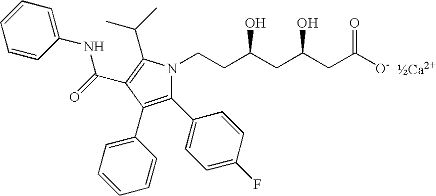

The specific isomer has been described in U.S. Pat. No. 5,273,995, herein incorporated by reference. In a preferred embodiment, the HMG-CoA reductase inhibitor is selected from the group consisting of atorvastatin, the cyclized lactone form of atorvastatin, a 2-hydroxy, 3-hydroxy or 4-hydroxy derivative of such compounds, and a pharmaceutically acceptable salt thereof.

In practice, use of the salt form amounts to use of the acid or lactone form. Appropriate pharmaceutically acceptable salts within the scope of the invention are those derived from bases such as sodium hydroxide, potassium hydroxide, lithium hydroxide, calcium hydroxide, 1-deoxy-2-(methylamino)-D-glucitol, magnesium hydroxide, zinc hydroxide, aluminum hydroxide, ferrous or ferric hydroxide, ammonium hydroxide or organic amines such as N-methylglucamine, choline, arginine and the like. Preferably, the lithium, calcium, magnesium, aluminum and ferrous or ferric salts are prepared from the sodium or potassium salt by adding the appropriate reagent to a solution of the sodium or potassium salt, i.e., addition of calcium chloride to a solution of the sodium or potassium salt of the compound of the formula A will give the calcium salt thereof.

### Methods of Treatment

The dosage forms of the present invention may be used to treat any condition, which is subject to treatment by administering a CETP inhibitor and an HMG-CoA reductase inhibitor, as disclosed in commonly assigned, copending U.S. Patent Application No. 2002/0035125A1, the disclosure of which is herein incorporated by reference.

In one aspect, the dosage forms of the present invention are used for antiatherosclerotic treatment.

In another aspect, the dosage forms of the present invention are used for slowing and/or arresting the progression of atherosclerotic plaques.

In another aspect, the dosage forms of the present invention are used for slowing the progression of atherosclerotic plaques in coronary arteries.

In another aspect, the dosage forms of the present invention are used for slowing the progression of atherosclerotic plaques in carotid arteries.

In another aspect, the dosage forms of the present invention are used for slowing the progression of atherosclerotic plaques in the peripheral arterial system.

In another aspect, the dosage form of the present invention, when used for treatment of atherosclerosis, causes the regression of atherosclerotic plaques.

In another aspect, the dosage forms of the present invention are used for regression of atherosclerotic plaques in coronary arteries.

In another aspect, the dosage forms of the present invention are used for regression of atherosclerotic plaques in carotid arteries.

In another aspect, the dosage forms of the present invention are used for regression of atherosclerotic plaques in the peripheral arterial system.

In another aspect, the dosage forms of the present invention are used for HDL elevation treatment and antihyperlipidemic treatment (including LDL lowering).

In another aspect, the dosage forms of the present invention are used for antianginal treatment.

In another aspect, the dosage forms of the present invention are used for cardiac risk management.

Other features and embodiments of the invention will become apparent from the following examples, which are given for illustration of the invention rather than for limiting its intended scope.

### Example 1

This example demonstrates a dosage form of the invention that provides a combination of immediate and controlled-release delivery of a solubility-improved form of the CETP inhibitor [2R,4S] 4-[(3,5-bis-trifluoromethyl-benzyl)-methoxycarbonyl-amino]-2-ethyl-6-trifluoromethyl-3,4-dihydro-2H-quinoline-1-carboxylic acid ethyl ester (torcetrapib), and immediate-release delivery of the HMG-CoA reductase inhibitor atorvastatin hemicalcium trihydrate (hereinafter termed “atorvastatin”).

### Formation of the Solubility-Improved Form of the CETP Inhibitor

A solubility-improved form of torcetrapib was prepared by forming a solid amorphous dispersion of torcetrapib in hydroxypropyl methyl cellulose acetate succinate (HPMCAS). The dispersion was prepared by spray-drying a solution containing 4.0 wt % torcetrapib, 12.0 wt % HPMCAS-MG (AQUOT-MG manufactured by Shin Etsu (Tokyo, Japan)), and 84 wt % acetone. The solution was spray-dried using a pressure spray nozzle (Delavan SDX III) at an atomization pressure of 48 atm (700 psig) with a liquid feed rate of about 100 kg/hour into the stainless steel chamber of a Niro PSD-4 spray-dryer maintained at a temperature of about 110° C. at the inlet and about 45° C. at the outlet. Secondary drying was performed using an Aeromatic MP-6 fluid bed dryer with a drying bed temperature of 40° C., and a drying time of 360 minutes.

### Controlled-Release CETP Inhibitor Composition

A bilayer osmotic controlled-release device was formed from the solubility-improved form of torcetrapib as follows. A drug-containing composition was formed by blending 30 wt % torcetrapib solid amorphous dispersion (25 wt % torcetrapib:HPMCAS-MG), 30 wt % polyethylene oxide (PEO) having an average molecular weight of 600,000, 39 wt % xylitol (trade name XYLITOL C granular), and 1 wt % magnesium stearate. First the drug containing composition, torcetrapib, PEO (pre-screened) and xylitol, were combined together. The formulation was then blended for 10 minutes in a TURBULA mixer. This blend was pushed through a 20 mesh screen (screen size of 850 microns), then blended again for 10 minutes in the same mixer. Next, the magnesium stearate was added and the drug-containing composition was blended again for 3 minutes in the same mixer.

A water-swellable composition was formed by blending the following materials: 65 wt % PEO having an average molecular weight of 5,000,000, 34.3 wt % of the tableting aid microcrystalline cellulose (trade name Avicel PH102), 0.5 wt % magnesium stearate, and 0.2 wt % Blue Lake #2. The PEO (pre-screened), Avicel, and Blue Lake dye were combined and blended for 10 minutes in a TURBULA mixer. All ingredients were pushed through a 20 mesh screen (screen size of 850 microns), then blended again for 10 minutes in the same mixer. Next, the magnesium stearate was added and the drug-containing composition was blended again for 3 minutes in the same mixer.

Tablet cores were formed using a F-press by placing 500 mg of the drug-containing composition in a standard 7/16 inch standard round concave (SRC) die and gently leveling with the press. Then 200 mg of the water-swellable composition was placed in the die on top of the drug-containing composition. The tablet core was then compressed to a hardness of about 15 kp. The resulting bi-layer tablet core has a total weight of 700 mg and contains a total of 5.36 wt % torcetrapib (37.5 mg), 16.07 wt % HPMCAS-MG, 27.86 wt % XYLITOL C granular, 21.43 wt % PEO 600,000, 18.57 wt % PEO 5,000,000, 9.8 wt % AVICEL PH102, 0.85 wt % magnesium stearate, and 0.06 wt % Blue Lake dye.

A water-permeable coating was applied to the core using a Vector LDCS-20 pan coater. The coating solution contains cellulose acetate (CA 398-10 from Eastman Fine Chemical, Kingsport, Tenn.), polyethylene glycol (PEG 3350, Dow Chemical), water, and acetone in a weight ratio of 3.5/1.5/4/91 (wt %). The flow rate of the inlet heated drying air of the pan coater was set at 35 CFM with an outlet temperature of 28° C. Air at 15 psig (2.1 SCFM) was used to atomize the coating solution from the spray nozzle, with a nozzle-to-bed distance of 2 inches. The pan rotation was set to 22 rpm. The so-coated tablets were dried at 40° C. for 16 hours in a convection oven removing essentially all of the acetone and water from the coating. The final dry coating weight (86.5 mg) amounted to 11.0 wt % of the tablet core, and consisted of about 60.6 mg of CA, and 25.9 mg PEG 3350. One 900 μm diameter hole was then mechanically drilled in the coating on the drug-containing composition side of the tablet to provide 1 delivery port per tablet.

### Immediate-Release CETP Inhibitor and Atorvastatin Coating

The osmotic controlled-release device above was coated with an immediate-release layer of torcetrapib solid amorphous dispersion (25 wt % torcetrapib:HPMCAS-MG) and atorvastatin by dipping each tablet in the following solution: 90.0 wt % water, 3.0 wt % Opadry clear (available from Colorcon, Inc., WestPoint, Pa.), 4.0% torcetrapib solid amorphous dispersion, and 3.0 wt % atorvastatin. The coating solution was formed by adding Opadry clear polymer to rapidly-stirring water and stirred at room temperature for about 1 hour. Next, atorvastatin and torcetrapib solid amorphous dispersion were added to the coating solution to form a suspension and the mixture was stirred about 30 minutes. Each tablet was dipped in the stirred suspension and dried with hot air on a screen before the tablet was coated again. Several coatings were applied to each tablet, and the tablets were dried overnight at room temperature before weighing to determine the total amount of immediate-release coating applied. An average of 79.1 mg of coating material (about 7.9 mg of torcetrapib and about 23.7 mg of atorvastatin) was applied to each tablet.

### In Vitro Dissolution Tests

In vitro tests were performed to measure the release of torcetrapib and atorvastatin from the dosage form of Example 1. To perform an in vitro dissolution test, each dosage form was first placed into a stirred USP type 1 dissoette flask containing 900 mL of a buffer solution simulating the contents of the intestine (5 mM KH₂PO₄, 1.5% wt. CTAC, pH 6.3). The solutions were stirred using baskets rotating at a rate of 200 rpm. Samples were taken at periodic intervals using an autosampling dissoette device programmed to periodically remove a sample of the receptor solution. The drug concentrations were analyzed by HPLC using a Zorbax SB-CN column, and a mobile phase of 56/44 (vol. %) acetonitrile/acetate buffer, pH 4 with 1.5% wt. CTAC. UV absorption was measured at 244 nm. Results are shown in Table 1.

TABLE 1

| | torcetrapib | atorvastatin |
|---|---|---|
| Time (hours) | (wt % released) | (wt % released)* |
| 0 | 0 | 0 |
| 0.5 | 15 | 73 |
| 1 | 21 | 94 |
| 2 | 23 | 97 |
| 4 | 30 | 99 |
| 8 | 53 | 97 |
| 12 | 78 | 99 |
| 18 | 94 | 99 |
| 24 | 98 | 100 |
| 36 | 99 | 100 |

*Atorvastatin values normalized to 36 hour value of 6.4 mg.

The data show that the dosage form of Example 1 provided immediate release of atorvastatin, providing 94% release in one hour. The atorvastatin wt % released values from 0.5 to 24 hours were normalized to the 36 hour value due to lower than expected potency of atorvastatin in the coating. The low potency was probably due to settling of atorvastatin in the coating suspension vessel during dip coating. In addition, the dosage form of Example 1 provided immediate and controlled release of the torcetrapib, with the time to release 70 wt % of the drug from the dosage form being about 11 hours. The dosage form released the torcetrapib at an average rate of 6.5 wt %/hr during the first 12 hours following administration to the test medium.

### Example 2

This example demonstrates a second dosage form of the invention that provides a combination of immediate and controlled-release delivery of a solubility-improved form of the CETP inhibitor [2R,4S] 4-[(3,5-bis-trifluoromethyl-benzyl)-methoxycarbonyl-amino]-2-ethyl-6-trifluoromethyl-3,4-dihydro-2H-quinoline-1-carboxylic acid ethyl ester (torcetrapib), and immediate-release delivery of the HMG-CoA reductase inhibitor atorvastatin hemicalcium trihydrate (hereinafter termed “atorvastatin”). The torcetrapib was in the form of a solid amorphous dispersion, made as described in Example 1.

Controlled-Release CETP Inhibitor Composition: The torcetrapib bilayer osmotic controlled-release device was made as described in Example 1.
Immediate-Release CETP Inhibitor Coating: The osmotic controlled-release device above was coated with an immediate-release layer of torcetrapib solid amorphous dispersion (25 wt % torcetrapib:HPMCAS-MG) by dipping each tablet in the following solution: 92.0 wt % water, 4.0 wt % Opadry clear (available from Colorcon, Inc., WestPoint, Pa.), and 4.0% torcetrapib solid amorphous dispersion. The coating solution was formed by adding Opadry clear polymer to rapidly-stirring water and stirring at room temperature for about 1 hour. Next, torcetrapib solid amorphous dispersion was added to the coating solution to form a suspension and the mixture was stirred about 30 minutes. Each tablet was dipped in the stirred suspension and dried with hot air on a screen before the tablet was coated again. Several coatings were applied to each tablet, and the tablets were dried overnight at room temperature before weighing to determine the total amount of immediate-release coating applied. An average of 67.1 mg of coating material (about 8.4 mg of torcetrapib) was applied to each tablet.
Immediate-Release Atorvastatin Coating: The osmotic controlled-release device with immediate-release torcetrapib coating above was coated with an immediate-release layer of atorvastatin by dipping each tablet in the following solution: 92.0 wt % water, 4.0 wt % Opadry® clear (available from Colorcon, Inc., WestPoint, Pa.), and 4.0 wt % atorvastatin. The coating solution was formed by adding Opadry clear polymer to rapidly-stirring water, and stirring at room temperature for about 1 hour. Next, atorvastatin was added to the coating solution to form a suspension and the mixture was stirred about 30 minutes. Each tablet was dipped in the stirred suspension and dried with hot air on a screen before the tablet was coated again. Several coatings were applied to each tablet, and the tablets were dried overnight at room temperature before weighing to determine the total amount of immediate-release coating applied. An average of 52.7 mg of coating material (about 26.4 mg of atorvastatin) was applied to each tablet.

### In Vitro Dissolution Tests

In vitro tests were performed as described in Example 1. Results are shown in Table 2.

TABLE 2

| | torcetrapib | atorvastatin |
|---|---|---|
| Time (hours) | (wt % released) | (wt % released)* |
| 0 | 0 | 0 |
| 0.5 | 7 | 75 |
| 1 | 12 | 95 |
| 2 | 14 | 99 |
| 4 | 19 | 99 |
| 8 | 44 | 98 |
| 12 | 64 | 98 |
| 18 | 84 | 99 |
| 24 | 89 | 99 |
| 36 | 91 | 100 |

*Atorvastatin values normalized to 36 hour value of 12.4 mg.

The data show that the dosage form of Example 2 provided immediate release of atorvastatin, providing 95% release in one hour. The atorvastatin wt % released values from 0.5 to 24 hours were normalized to the 36 hour value due to lower than expected potency of atorvastatin in the coating. The low potency was probably due to settling of atorvastatin in the coating suspension vessel during dip coating. In addition, the dosage form of Example 2 provided immediate and controlled release of the torcetrapib, with the time to release 70 wt % of the drug from the dosage form being about 13 hours. The dosage form released the torcetrapib at an average rate of 5.3 wt %/hr during the first 12 hours following administration to the test medium.

### Example 3

This example demonstrates a third dosage form of the invention that provides a combination of immediate and controlled-release delivery of a solubility-improved form of the CETP inhibitor [2R,4S] 4-[(3,5-bis-trifluoromethyl-benzyl)-methoxycarbonyl-amino]-2-ethyl-6-trifluoromethyl-3,4-dihydro-2H-quinoline-1-carboxylic acid ethyl ester (torcetrapib), and immediate-release delivery of the HMG-CoA reductase inhibitor atorvastatin hemicalcium trihydrate (hereinafter termed “atorvastatin”). The torcetrapib was in the form of a solid amorphous dispersion, made as described in Example 1.

Controlled-Release Device: An osmotic controlled-release device comprising the solid amorphous dispersion of torcetrapib in HPMCAS-MG was prepared as follows. A mixture was prepared containing 25.0 wt % of the torcetrapib:HPMCAS-MG dispersion of Example 1, 64.5 wt % sorbitol (NEOSORB P110, available from Roquette), 8.0 wt % hydroxyethylcellulose (NATROSOL 250HX, available from Hercules), 1.5% sodium lauryl sulfate (SLS), and 1.0 wt % magnesium stearate. Torcetrapib, soribitol (pre-screened), hydroxyethylcellulose and SLS were blended for 10 minutes in a TURBULA mixer, pushed through a 20-mesh screen, and then blended again for 10 minutes in the same mixer. Next, magnesium stearate was added and the composition was blended again for 3 minute in the same mixer. Tablet cores were formed by placing 600 mg of the tablet mixture in a caplet die (0.87 cm×1.73 cm [0.343×0.6807 inch]) and compressed using an F-press to a hardness of 14 kp. A water-permeable coating was applied as described in Example 1 using a Vector LDCS-20 pan coater. The coating solution contains CA 398-10, PEG 3350, water, and acetone in a weight ratio of 412/5/89. The final dry coating weight was 5.6 wt % of the tablet core (35.7 mg, comprising about 23.8 mg CA and about 11.9 mg PEG 3350), and one 900 μm diameter hole was mechanically-drilled in the coating to provide a delivery port. The delivery port was drilled at one end of the caplet at approximately the point where the longest axis through the caplet intersects the caplet surface. The monolayer osmotic controlled-release device contains 37.5 mg of torcetrapib.
Immediate-Release CETP Inhibitor and Atorvastatin Coating: Immediate-release CETP Inhibitor and Atorvastatin coating was made and applied as described in Example 1. An average of 79.3 mg of coating material (about 7.9 mg of torcetrapib and about 23.8 mg of atorvastatin) was applied to each tablet.

### In Vitro Dissolution Tests

In vitro tests were performed as described in Example 1. Results are shown in Table 3.

TABLE 3

| | torcetrapib | atorvastatin |
|---|---|---|
| Time (hours) | (wt % released) | (wt % released)* |
| 0 | 0 | 0 |
| 0.5 | 13 | 82 |
| 1 | 19 | 96 |
| 2 | 24 | 98 |
| 4 | 33 | 97 |
| 8 | 57 | 98 |
| 12 | 72 | 98 |
| 18 | 84 | 98 |
| 24 | 92 | 99 |
| 36 | 97 | 100 |

*Atorvastatin values normalized to 36 hour value of 6.3 mg.

The data show that the dosage form of Example 3 provided immediate release of atorvastatin, providing 96% release in one hour. The atorvastatin wt % released values from 0.5 to 24 hours were normalized to the 36 hour value due to lower than expected potency of atorvastatin in the coating. The low potency was probably due to settling of atorvastatin in the coating suspension vessel during dip coating. In addition, the dosage form of Example 3 provided immediate and controlled release of the torcetrapib, with the time to release 70 wt % of the drug from the dosage form being about 12 hours. The dosage form released the torcetrapib at an average rate of 6.0 wt %/hr during the first 12 hours following administration to the test medium.

### Example 4

This example demonstrates a forth dosage form of the invention that provides a combination of immediate and controlled-release delivery of a solubility-improved form of the CETP inhibitor [2R,4S] 4-[(3,5-bis-trifluoromethyl-benzyl)-methoxycarbonyl-amino]-2-ethyl-6-trifluoromethyl-3,4-dihydro-2H-quinoline-1-carboxylic acid ethyl ester (torcetrapib), and immediate-release delivery of the HMG-CoA reductase inhibitor atorvastatin hemicalcium trihydrate (hereinafter termed “atorvastatin”). The torcetrapib was in the form of a solid amorphous dispersion, made as described in Example 1.

Controlled-Release Device: The torcetrapib monolayer osmotic controlled-release device was made as described in Example 3.
Immediate-Release CETP Inhibitor Coating: The immediate-release CETP inhibitor coating was made and applied as described in Example 2. An average of 72.2 mg of coating material (about 9.0 mg of torcetrapib) was applied to each tablet.
Immediate-Release Atorvastatin Coating: The immediate-release atorvastatin coating was made and applied as described in Example 2. An average of 42.4 mg of coating material (about 21.2 mg of atorvastatin) was applied to each tablet.

### In Vitro Dissolution Tests

In vitro tests were performed as described in Example 1. Results are shown in Table 4.

TABLE 4

| | torcetrapib | atorvastatin |
|---|---|---|
| Time (hours) | (wt % released) | (wt % released)* |
| 0 | 0 | 0 |
| 0.5 | 12 | 79 |
| 1 | 17 | 93 |
| 2 | 21 | 96 |
| 4 | 29 | 97 |
| 8 | 51 | 98 |
| 12 | 68 | 98 |
| 18 | 81 | 98 |
| 24 | 90 | 99 |
| 36 | 95 | 100 |

*Atorvastatin values normalized to 36 hour value of 9.2 mg.

The data show that the dosage form of Example 4 provided immediate release of atorvastatin, providing 93% release in one hour. The atorvastatin wt % released values from 0.5 to 24 hours were normalized to the 36 hour value due to lower than expected potency of atorvastatin in the coating. The low potency was probably due to settling of atorvastatin in the coating suspension vessel during dip coating. In addition, the dosage form of Example 4 provided immediate and controlled release of the torcetrapib, with the time to release 70 wt % of the drug from the dosage form being about 12 hours. The dosage form released the torcetrapib at an average rate of 5.6 wt %/hr during the first 12 hours following administration to the test medium.

### Example 5

This example demonstrates a fifth dosage form of the invention that provides a combination of immediate and controlled-release delivery of a solubility-improved form of the CETP inhibitor [2R,4S] 4-[(3,5-bis-trifluoromethyl-benzyl)-methoxycarbonyl-amino]-2-ethyl-6-trifluoromethyl-3,4-dihydro-2H-quinoline-1-carboxylic acid ethyl ester (torcetrapib), and immediate-release delivery of the HMG-CoA reductase inhibitor atorvastatin hemicalcium trihydrate (hereinafter termed “atorvastatin”). The torcetrapib was in the form of a solid amorphous dispersion, made as described in Example 1. A bilayer matrix immediate and controlled-release device was formed from the solubility-improved form of torcetrapib as follows.

Controlled-Release CETP Inhibitor Composition: A controlled-release composition was formed by blending 57.7 wt % torcetrapib solid amorphous dispersion (25 wt % torcetrapib:HPMCAS-MG), 25 wt % hydroxypropyl methyl cellulose (trade name Methocel K100M), 16.3 wt % microcrystalline cellulose (trade name Avicel PH102), and 1 wt % magnesium stearate. The drug-containing composition ingredients were first combined without the magnesium stearate and blended for 10 minutes in a TURBULA mixer. This blend was pushed through a 20-mesh screen (screen size of 850 microns), then blended again for 10 minutes in the same mixer. Next, the magnesium stearate was added and the drug-containing composition was blended again for 3 minutes in the same mixer.
Immediate-Release CETP Inhibitor Composition: An immediate-release CETP Inhibitor composition was formed by combining 36.0 wt % torcetrapib solid amorphous dispersion (25 wt % torcetrapib:HPMCAS-MG), 58.0 wt % microcrystalline cellulose (trade name Avicel PH102), 5% crospovidone (trade name Polyplasdone) and 1 wt % magnesium stearate. The drug-containing composition ingredients were first combined without the magnesium stearate and blended for 10 minutes in a TURBULA mixer. All ingredients were pushed through a 20-mesh screen (screen size of 850 microns), and then blended again for 10 minutes in the same mixer. Next, the magnesium stearate was added and the drug-containing composition was blended again for 3 minutes in the same mixer.
Immediate-Release Atorvastatin Granulation Composition: An immediate-release granulation of atorvastatin was made by combining 13.9 wt % atorvastatin trihydrate hemicalcium salt, 42.3 wt % calcium carbonate, 17.7 wt % microcrystalline cellulose, 3.8 wt % croscarmellose sodium, 0.5 wt % polysorbate 80, 2.6 wt % hydroxypropyl cellulose, and 19.2 wt % pregelatinized starch. The composition ingredients were combined and blended for 10 minutes in a TURBULA mixer. All ingredients were pushed through a 20-mesh screen (screen size of 850 microns) and then blended again for 10 minutes in the same mixer.
Immediate-release CETP Inhibitor and Atorvastatin Blend Composition: 32.0 wt % of the Immediate-release composition of CETP Inhibitor granulation and about 67.0 wt % Atorvastatin were then blended for 10 minutes in a TURBULA mixer. Next 1.0% magnesium stearate was added and the drug-containing composition was blended again for 3 minutes in the same mixer.

Bilayer tablets were formed using a F-press by placing 260 mg of Controlled-Release CETP Inhibitor Composition in a standard 13/32 inch standard round concave (SRC) die and gently leveling with the press. Then, 260 mg of the immediate-release composition of CETP Inhibitor and atorvastatin was placed in the die on top of the Controlled-Release CETP Inhibitor Composition. The tablet core was then compressed to a hardness of about 18 Kp. The resulting bi-layer tablet core had a total weight of 520 mg and contained a total of 45 mg torcetrapib (37.5 mg controlled-release/7.5 mg immediate-release) and immediate-release atorvastatin (22 mg).

### In Vitro Dissolution Tests

In vitro tests were performed as described in Example 1. Results are shown in Table 5.

TABLE 5

| | torcetrapib | atorvastatin |
|---|---|---|
| Time (hours) | (wt % released) | (wt % released) |
| 0 | 0 | 0 |
| 0.5 | 17 | 83 |
| 1 | 18 | 91 |
| 2 | 21 | 92 |
| 4 | 27 | 92 |
| 8 | 41 | 92 |
| 12 | 57 | 92 |
| 18 | 76 | 93 |
| 24 | 91 | 93 |
| 36 | 100 | 93 |

The data show that the dosage form of Example 5 provided immediate release of atorvastatin, providing 91% release in one hour. In addition, the dosage form of Example 5 provided immediate and controlled release of the torcetrapib, with the time to release 70 wt % of the drug from the dosage form being about 17 hours. The dosage form released the torcetrapib at an average rate of 4.8 wt %/hr during the first 12 hours following administration to the test medium.

### Example 6

This example demonstrates a sixth dosage form of the invention that provides a combination of immediate and controlled-release delivery of a solubility-improved form of the CETP inhibitor [2R,4S] 4-[(3,5-bis-trifluoromethyl-benzyl)-methoxycarbonyl-amino]-2-ethyl-6-trifluoromethyl-3,4-dihydro-2H-quinoline-1-carboxylic acid ethyl ester (torcetrapib), and immediate-release delivery of the HMG-CoA reductase inhibitor atorvastatin hemicalcium trihydrate (hereinafter termed “atorvastatin”). The torcetrapib was in the form of a solid amorphous dispersion, made as described in Example 1. A trilayer matrix immediate and controlled-release device was formed from the solubility-improved form of torcetrapib as follows.

Controlled-Release CETP Inhibitor Composition: A controlled-release CETP inhibitor composition was made as described in Example 5.

Immediate-Release CETP Inhibitor Composition: An immediate-release CETP Inhibitor composition was formed by combining 36.0 wt % torcetrapib solid amorphous dispersion (25 wt % torcetrapib:HPMCAS-MG), 58.0 wt % microcrystalline cellulose (trade name Avicel PH102), 5% crospovidone (trade name Polyplasdone) and 1 wt % magnesium stearate. The drug-containing composition ingredients were first combined without the magnesium stearate and blended for 10 minutes in a TURBULA mixer. All ingredients were pushed through a 20-mesh screen (screen size of 850 microns), and then blended again for 10 minutes in the same mixer. Next, the magnesium stearate was added and the drug-containing composition was blended again for 3 minutes in the same mixer. Immediate-Release Atorvastatin Granulation Composition: An immediate-release granulation of atorvastatin was made by combining 13.9 wt % atorvastatin trihydrate hemicalcium salt, 42.3 wt % calcium carbonate, 17.7 wt % microcrystalline cellulose, 3.8 wt % croscarmellose sodium, 0.5 wt % polysorbate 80, 2.6 wt % hydroxypropyl cellulose, and 19.2 wt % pregelatinized starch. The composition ingredients were combined and blended for 10 minutes in a TURBULA mixer. All ingredients were pushed through a 20-mesh screen (screen size of 850 microns) and then blended again for 10 minutes in the same mixer.

Trilayer tablets were formed using a F-press by placing 260 mg of controlled-release CETP Inhibitor composition in a standard 13/32 inch standard round concave (SRC) die and gently leveling with the press. Then, 83.4 mg of the immediate-release composition of CETP Inhibitor was placed in the die and gently leveled with the press. Then, 174 mg of the atorvastatin composition was placed in the die on top of the controlled-release and immediate-release CETP Inhibitor compositions. The tablet core was then compressed to a hardness of about 18 Kp. The resulting tri-layer tablet core has a total weight of 520 mg and contains a total of 45 mg torcetrapib (37.7 mg Controlled-Release/7.5 mg immediate-release) and immediate-release atorvastatin (22 mg).

### In Vitro Dissolution Tests

In vitro tests were performed as described in Example 1. Results are shown in Table 6.

TABLE 6

| | torcetrapib | atorvastatin |
|---|---|---|
| Time (hours) | (wt % released) | (wt % released) |
| 0 | 0 | 0 |
| 0.5 | 16 | 88 |
| 1 | 18 | 92 |
| 2 | 20 | 92 |
| 4 | 26 | 93 |
| 8 | 38 | 94 |
| 12 | 51 | 93 |
| 18 | 67 | 94 |
| 24 | 80 | 94 |
| 36 | 95 | 94 |

The data show that the dosage form of Example 6 provided immediate release of atorvastatin, providing 92% release in one hour. In addition, the dosage form of Example 6 provided immediate and controlled release of the torcetrapib, with the time to release 70 wt % of the drug from the dosage form being about 20 hours. The dosage form released the torcetrapib at an average rate of 4.3 wt %/hr during the first 12 hours following administration to the test medium.

### Example 7

This example demonstrates a seventh dosage form of the invention that provides a combination of immediate and controlled-release delivery of a solubility-improved form of the CETP inhibitor [2R,4S] 4-[(3,5-bis-trifluoromethyl-benzyl)-methoxycarbonyl-amino]-2-ethyl-6-trifluoromethyl-3,4-dihydro-2H-quinoline-1-carboxylic acid ethyl ester (torcetrapib), and immediate-release delivery of the HMG-CoA reductase inhibitor atorvastatin hemicalcium trihydrate (hereinafter termed “atorvastatin”). The torcetrapib in the immediate-release composition was in the form of a solid amorphous dispersion, made as described in Example 1. A capsule device containing controlled-release torcetrapib spray-layered multiparticulates which results in a solubility-improved form of torcetrapib, immediate-release torcetrapib from the solubility-improved form of torcetrapib made as described in Example 1, and immediate-release atorvastatin as follows.

### Formation of the Solubility-Improved Controlled-Release CETP Inhibitor Composition

A solubility-improved form of torcetrapib was prepared by forming a solid amorphous dispersion of torcetrapib in hydroxypropyl methyl cellulose acetate succinate (HPMCAS) and cellulose acetate (CA). The dispersion was prepared by spray-layering a solution containing 4.0 wt % torcetrapib, 12.0 wt % HPMCAS-MG (AQUOT-MG manufactured by Shin Etsu (Tokyo, Japan)), 0.3% wt cellulose acetate (CA 398-10 from Eastman Fine Chemical, Kingsport, Tenn.), and 83.7 wt % acetone. The solution was spray-layered onto 35-40 mesh sugar beads (Paulaur, Cranbury, N.J.) using a F-GPCG-1 fluidized bed coater (Glatt, Germany) equipped with appropriate air distribution plate and Wurster (bottom spray) insert. Sugar bead cores were fluidized utilizing a nitrogen purge and the drug containing solution was sprayed at the following conditions. Atomization air pressure of 2 bar, liquid feed rate of 7 g/min, product and outlet temperature of 30-31° C. Secondary drying was performed using convection tray dryer with a drying bed temperature of 40° C. for 18 hours.
Immediate-Release CETP Inhibitor Composition: An immediate-release CETP inhibitor composition was made as described in Example 6.
Immediate-Release Atorvastatin Granulation Composition: An immediate-release atorvastatin granulation composition was made as described in Example 6.
Dosage Form of the Invention: To prepare each dosage form of Example 7, a Quali-V HPMC capsule (available from Shionogi), size 00, was filled with 399.6 mg torcetrapib controlled-release composition described above, 83.4 mg of the immediate-release torcetrapib granulation, and 174.0 mg of atorvastatin granulation. The final dosage form contained 37.5 mg of controlled-release torcetrapib, 7.5 mg of immediate-release torcetrapib, and 22 mg of immediate-release atorvastatin.

### In Vitro Dissolution Tests

In vitro tests were performed as described in Example 1. Results are shown in Table 7.

TABLE 7

| | torcetrapib | atorvastatin |
|---|---|---|
| Time (hours) | (wt % released) | (wt % released) |
| 0 | 0 | 0 |
| 0.5 | 36 | 76 |
| 1 | 42 | 88 |
| 2 | 51 | 87 |
| 4 | 67 | 87 |
| 8 | 85 | 88 |
| 12 | 97 | 88 |
| 18 | 104 | 88 |
| 24 | 104 | 89 |

The data show that the dosage form of Example 7 provided immediate release of atorvastatin, providing 88% release in one hour. In addition, the dosage form of Example 7 provided immediate and controlled release of the torcetrapib, with the time to release 70 wt % of the drug from the dosage form being about 4 hours. The dosage form released the torcetrapib at an average rate of 8.0 wt %/hr during the first 12 hours following administration to the test medium.

Throughout this application, various publications are referenced. The disclosures of these publications in their entireties are hereby incorporated by reference into this application for all purposes.

It will be apparent to those skilled in the art that various modifications and variations may be made in the present invention without departing from the scope or spirit of the invention. Other embodiments of the invention will be apparent to those skilled in the art from consideration of the specification and practice of the invention disclosed herein. It was intended that the specification and examples be considered as exemplary only, with a true scope and spirit of the invention being indicated by the following claims.

1. A dosage form comprising:
(a) a cholesteryl ester transfer protein inhibitor in a solubility-improved form, and
(b) an HMG-CoA reductase inhibitor;
wherein said HMG-CoA reductase is in immediate release form and a portion of said cholesteryl ester transfer protein inhibitor is in immediate release form and a portion of said cholesteryl ester transfer protein inhibitor is in controlled release form.

2. The dosage form of claim 1 wherein said dosage form is a sustained release dosage form that releases in vivo or in vitro less than about 70 wt % of said cholesteryl ester transfer protein inhibitor over about 2 hours or more after administration of said dosage form to an aqueous environment of use.

3. The dosage form of claim 1 wherein, following administration to an in vivo use environment, said dosage form provides one or more of:
(i) at least about 50% inhibition of plasma cholesteryl ester transfer protein for at least about 12 hours;
(ii) a maximum drug concentration in the blood that is less than or equal to about 80% of the maximum drug concentration in the blood provided by a dosage form that provides immediate release of the same amount of said solubility-improved form of said cholesteryl ester transfer protein inhibitor;
(iii) a mean HDL cholesterol level after dosing for 8 weeks that is at least about 1.2-fold that obtained prior to dosing; and
(iv) a mean LDL cholesterol level after dosing for 8 weeks that is less than or equal to about 90% that obtained prior to dosing.

4. The dosage form of claim 3 wherein, following administration to an in vivo use environment, said dosage form provides two or more of:
(i) at least about 50% inhibition of plasma cholesteryl ester transfer protein for at least about 12 hours;
(ii) a maximum drug concentration in the blood that is less than or equal to about 80% of the maximum drug concentration in the blood provided by a dosage form that provides immediate release of the same amount of said solubility-improved form of said cholesteryl ester transfer protein inhibitor;
(iii) a mean HDL cholesterol level after dosing for 8 weeks that is at least about 1.2-fold that obtained prior to dosing; and
(iv) a mean LDL cholesterol level after dosing for 8 weeks that is less than or equal to about 90% that obtained prior to dosing.

5. The dosage form of claim 1 wherein said dosage form comprises said cholesteryl ester transfer protein inhibitor in the form of a matrix controlled-release device.

6. The dosage form of claim 1 wherein said dosage form comprises said cholesteryl ester transfer protein inhibitor in the form of an osmotic controlled-release device.

7. The dosage form of claim 1 wherein said dosage form comprises a plurality of controlled-release multiparticulates comprising said controlled release portion of cholesteryl ester transfer protein inhibitor.

8. The dosage form of claim 1 wherein said dosage form comprises an immediate release composition comprising said HMG-CoA reductase inhibitor.

9. The dosage form of claim 8 wherein said immediate release composition further comprises said immediate release portion of cholesteryl ester transfer protein inhibitor.

10. The dosage form of claim 1 wherein said dosage form comprises a kit.

11. The dosage form of claim 1 wherein said cholesteryl ester transfer protein inhibitor is torcetrapib.

12. The dosage form of claim 1 wherein said HMG-CoA reductase inhibitor is atorvastatin hemicalcium trihydrate.

13. The dosage form of claim 1 comprising torcetrapib and atorvastatin, or pharmaceutically acceptable forms thereof.

14. The dosage form of claim 11 wherein, following administration to an in vivo use environment, said dosage form provides a plasma concentration of said torcetrapib of about 70 ng/mL or more for a period of about 12 hour or more.

15. The dosage form of claim 1 wherein said solubility-improved form is a solid amorphous dispersion comprising said cholesteryl ester transfer protein inhibitor and a polymer.
